SEQUENCE LISTING (75 sequences)

SEQ ID NO: 1 - protein, 19 aa, Mus musculus
  source 1..19: note = 2291.014.055.067 (fel d1 MAb2)_cAb10204
MNFGLSLIFLVLILKGVQC

SEQ ID NO: 2 - protein, 19 aa, Mus musculus
  source 1..19: note = 2293.254.030 (Fel d1 MAb9)_cAb10205
MGWSYIILFLVATATDVHS

SEQ ID NO: 3 - protein, 19 aa, Mus musculus
  source 1..19: note = 2297.167.064 (Fel d1 MAb18)_cAb10206
MEWNWVVLFLLSLTAGVYA

SEQ ID NO: 4 - protein, 19 aa, Mus musculus
  source 1..19: note = 2297.179.089 (Fel d1 MAb20)_cAb10207
MGWSCIILFLVATATGVHS

SEQ ID NO: 5 - protein, 19 aa, synthetic construct
  source 1..19: note = 2297.179.089 (Fel d1 MAb20-1)_cAb10207
MDWLWNLLFLMAAAQSIQA

SEQ ID NO: 6 - protein, 20 aa, Mus musculus
  source 1..20: note = 2291.014.055.067 (fel d1 MAb2)_cAb10204
MRVLAELLGLLLFCFLGVRC

SEQ ID NO: 7 - protein, 20 aa, Mus musculus
  source 1..20: note = 2293.254.030 (Fel d1 MAb9)_cAb10205
MESQTQVLMSLLFWVSGTCG

SEQ ID NO: 8 - protein, 20 aa, Mus musculus
  source 1..20: note = 2297.167.064 (Fel d1 MAb18)_cAb10206
MSVLTQVLALLLLWLTGARC

SEQ ID NO: 9 - protein, 20 aa, Mus musculus
  source 1..20: note = 2297.179.089 (Fel d1 MAb20)_cAb10207
MESQTLVFISILLWLYGADG

SEQ ID NO: 10 - protein, 118 aa, Mus musculus
  source 1..118: note = 2291.014.055.067 (fel d1 MAb2)_cAb10204
EVKLMESGGGLVKPGGSLKLSCAASGFTFSSYDMSWVRQTPEKRLEWVATISSGGTYTYYPDSLKGRFTISRDNARNTLYLQMSSLRSEDTALFYCARLLRTYAMDYWGQGTSVTVSS

SEQ ID NO: 11 - protein, 119 aa, Mus musculus
  source 1..119: note = 2293.254.030 (Fel d1 MAb9)_cAb10205
QVQLQQPGAELVKPGASVKLSCKASGYTFTSYWMYWVKQRPGQGLEWIGEINPSNGRTNYNEKFKTKATLTVDKSSSTAYMQLSSLTSEDSAVYYCARRATMIGGFAYWGQGTLVTVSA

SEQ ID NO: 12 - protein, 120 aa, Mus musculus
  source 1..120: note = 2297.167.064 (Fel d1 MAb18)_cAb10206
QGQMQQSGAELVKPGASVKLSCKTSGFTFSSSYISWLKQKPGQSLEWIAWIYAGTGGTTYNQRFTGKAQLTVDSSSSTAYMHFSSLTTDDSAIYYCARHYGNPPAWFAYWGQGTLVTVSA

SEQ ID NO: 13 - protein, 121 aa, Mus musculus
  source 1..121: note = 2297.179.089 (Fel d1 MAb20)_cAb10207
QVQLQQPGTELVRPGASVKLSCKASGYTFTSYWINWVKQRPGQGLEWIGNIYPSDSYTNYNQKFKDKATLTVDKSSSTAYMQLSSPTSDDSAVYYCTRRGNYGNLYYFDYWGQGTTLTVSS

SEQ ID NO: 14 - protein, 118 aa, synthetic construct
  source 1..118: note = 2297.179.089 (Fel d1 MAb20-1)_cAb10207
QIQLVQSGPELRKPGETVRISCKASGYTFTTYGMDWVKQAPGKVLKWVGWINTNTGEPTYAEEFKGRFAFSLETSASTAYFQINNLKNEDTATYFCARKWLRRAMDYWGQGTSVTVSS

SEQ ID NO: 15 - protein, 108 aa, Mus musculus
  source 1..108: note = 2291.014.055.067 (fel d1 MAb2)_cAb10204
DIQMNQSPSSLSASLGDTITITCHASQNIIVWLNWYQQKPGNIPKLLIYKASNLHTGVPSRFSGSGSGTGFTLTISSLQPEDIATYYCQQGQSYHTWTFGGGTKLEIK

SEQ ID NO: 16 - protein, 113 aa, Mus musculus
  source 1..113: note = 2293.254.030 (Fel d1 MAb9)_cAb10205
DIVMTQSPSSLTVTTGEKVTMNCKSSQSLLNSANQKNYLTWHQQKPGQPPKLLIYWASTRESGVPDRFTGSGSGTDFTLTISSVQAEDLAVYYCQNDYSFPFTFGSGTKLEIK

SEQ ID NO: 17 - protein, 107 aa, Mus musculus
  source 1..107: note = 2297.167.064 (Fel d1 MAb18)_cAb10206
DIQMTQSPASLSASVGETVTITCRASGNIHNYLAWFQQRQGKSPQLLVYNAETLADGVPSRFSGSGSGTQYSLKISSLQPEDFGSYFCQHFWSRPPTFGGGTKLEIK

SEQ ID NO: 18 - protein, 107 aa, Mus musculus
  source 1..107: note = 2297.179.089 (Fel d1 MAb20)_cAb10207
NIVMTQSPKSMSMSVGERVTLSCKASENVGSYVFWYQQKPDQSPKLLIYGASNRYTGVPDRFTGSRSATDFTLTISNVQAEDLADYHCGQSYSYPLTFGAGTKLELK

SEQ ID NO: 19 - protein, 324 aa, Mus musculus
  source 1..324: note = 2291.014.055.067 (fel d1 MAb2)_cAb10204
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 118 aa, Synthetic construct; identical to SEQ ID NO: 14
  source 1..118: note = 2297.179.089 (Fel d1 MAb20-1)_cAb10207

SEQ ID NO: 21 - protein, 107 aa, Mus musculus
  source 1..107: note = 2291.014.055.067 (fel d1 MAb2)_cAb10204
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - DNA, 57 bp, Mus musculus
  source 1..57: mol_type = other DNA; note = 2291.014.055.067 (fel d1 MAb2)_cAb10204
atgaactttgggctgagcttgattttccttgtcctaattttaaaaggtgtccagtgt

SEQ ID NO: 23 - DNA, 57 bp, Mus musculus
  source 1..57: mol_type = other DNA; note = 2293.254.030 (Fel d1 MAb9)_cAb10205
atgggatggagctatatcatcctctttttggtagcaacagctacagatgtccactcc

SEQ ID NO: 24 - DNA, 57 bp, Mus musculus
  source 1..57: mol_type = other DNA; note = 2297.167.064 (Fel d1 MAb18)_cAb10206
atggaatggaactgggtcgttctcttcctcctgtcattaactgcaggtgtctatgcc

SEQ ID NO: 25 - DNA, 57 bp, Mus musculus
  source 1..57: mol_type = other DNA; note = 2297.179.089 (Fel d1 MAb20)_cAb10207
atgggatggagctgtatcatcctcttcttggtagcaacagctacaggtgtccactcc

SEQ ID NO: 26 - DNA, 57 bp, Synthetic construct
  source 1..57: mol_type = other DNA; note = 2297.179.089 (Fel d1 MAb20)_cAb10207
atggattggctgtggaacttgctattcctgatggcagctgcccaaagtatccaagca

SEQ ID NO: 27 - DNA, 60 bp, Mus musculus
  source 1..60: mol_type = other DNA; note = 2291.014.055.067 (fel d1 MAb2)_cAb10204
atgagggtccttgctgagctcctggggctgctgctgttctgctttttaggtgtgagatgt

SEQ ID NO: 28 - DNA, 60 bp, Mus musculus
  source 1..60: mol_type = other DNA; note = 2293.254.030 (Fel d1 MAb9)_cAb10205
atggaatcacagactcaggtcctcatgtccctgctgttctgggtatctggtacctgtggg

SEQ ID NO: 29 - DNA, 60 bp, Mus musculus
  source 1..60: mol_type = other DNA; note = 2297.167.064 (Fel d1 MAb18)_cAb10206
atgagcgtgctcactcaggtcctggcgttgctgctgctgtggcttacaggtgccagatgt

SEQ ID NO: 30 - DNA, 60 bp, Mus musculus
  source 1..60: mol_type = other DNA; note = 2297.179.089 (Fel d1 MAb20)_cAb10207
atggaatcacagactctggtcttcatatccatactgctctggttatatggtgctgatggg

SEQ ID NO: 31 - DNA, 354 bp, Mus musculus
  source 1..354: mol_type = other DNA; note = 2291.014.055.067 (fel d1 MAb2)_cAb10204
gaggtgaagctgatggagtctgggggaggcttagtgaagcctggagggtccctgaaactctcctgtgcagcctctggattcactttcagtagttatgacatgtcttgggttcgccagactccggagaagaggctggagtgggtcgcaaccattagtagtggtggtacttacacctactatccagacagtctaaagggccgattcaccatctccagagacaatgccaggaacaccctgtacctgcaaatgagcagtctgaggtctgaggacacggccttgttttactgtgcaagactacttcggacctatgctatggactactggggtcaaggaacctcagtcaccgtctcctca

SEQ ID NO: 32 - DNA, 357 bp, Mus musculus
  source 1..357: mol_type = other DNA; note = 2293.254.030 (Fel d1 MAb9)_cAb10205
caggtccaactgcagcagcctggggctgaactggtgaagcctggggcttcagtgaagctgtcctgcaaggcttctggctacaccttcaccagctactggatgtactgggtaaaacagaggcctggacaaggccttgagtggattggagagattaatcctagcaacggtcgtactaactacaatgagaagttcaagaccaaggccacactgactgtagacaaatcctccagcacagcctacatgcaactcagcagcctgacatctgaggactctgcggtctattactgtgcaagacgagctactatgattggggggtttgcttactggggccaagggactctggtcactgtctctgca

SEQ ID NO: 33 - DNA, 360 bp, Mus musculus
  source 1..360: mol_type = other DNA; note = 2297.167.064 (Fel d1 MAb18)_cAb10206
cagggtcagatgcagcagtctggagctgagctggtgaagcctggggcttcagtgaaactgtcctgcaagacttctggcttcaccttcagcagtagttatataagttggttgaagcaaaagcctggacagagtcttgagtggattgcatggatttatgctggaactggtggaactacctataatcagaggttcacaggcaaggcccaactgactgtagactcatcctccagcacagcctacatgcatttcagcagcctgacaactgatgactctgccatctattactgtgcaagacactatggtaacccccccgcctggtttgcttactggggccaagggactctggtcactgtctctgca

SEQ ID NO: 34 - DNA, 363 bp, Mus musculus
  source 1..363: mol_type = other DNA; note = 2297.179.089 (Fel d1 MAb20)_cAb10207
caggtccaactgcagcagcctgggactgagttggtgaggcctggggcttcagtgaagctgtcctgcaaggcttctggctacaccttcaccagctactggataaactgggtgaagcagaggcctggacaaggccttgagtggatcggaaatatttatccttctgatagttatactaactacaatcaaaagttcaaggacaaggccacattgactgtagacaaatcctccagtacagcctacatgcagctcagcagcccgacatctgacgactctgcggtctattactgtacaagaaggggcaactatggtaacctatactactttgactactggggccaaggcaccactctcacagtctcctca

SEQ ID NO: 35 - DNA, 354 bp, synthetic construct
  source 1..354: mol_type = other DNA; note = 2297.179.089 (Fel d1 MAb20)_cAb10207
cagatccagttggtgcagtctggacctgagctgaggaagcctggagagacagtcaggatctcctgcaaggcttctgggtataccttcacaacctatggaatggactgggtgaagcaggctccaggaaaggttttaaagtgggtgggctggataaacaccaacactggagagccaacatatgctgaagagttcaagggacggtttgccttctctttggaaacctctgccagcactgcctatttccagatcaacaacctcaaaaatgaggacacggctacatatttctgtgcaagaaaatggttacgacgggctatggactactggggtcaaggaacctcagtcaccgtctcctca

SEQ ID NO: 36 - DNA, 324 bp, Mus musculus
  source 1..324: mol_type = other DNA; note = 2291.014.055.067 (fel d1 MAb2)_cAb10204
gacatccagatgaaccagtctccatccagtctgtctgcatcccttggagacacaattaccatcacttgccatgccagtcagaacattattgtttggttaaactggtaccagcagaaaccaggaaatattcctaaactattgatctataaggcttccaacttgcacacaggcgtcccatcaaggtttagtggcagtggatctggaacaggtttcacattgaccatcagcagcctgcagcctgaagacattgccacttactactgtcaacagggtcaaagttatcacacgtggacgttcggtggaggcaccaagctggaaatcaaa

SEQ ID NO: 37 - DNA, 339 bp, Mus musculus
  source 1..339: mol_type = other DNA; note = 2293.254.030 (Fel d1 MAb9)_cAb10205
gacattgtgatgacacagtctccatcctccctgactgtgacaacaggagagaaggtcactatgaactgcaagtccagtcagagtctgttaaacagtgcaaatcaaaagaactacttgacctggcaccagcagaaaccaggtcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttttccattcacgttcggctcggggacaaagttggaaataaaa

SEQ ID NO: 38 - DNA, 321 bp, Mus musculus
  source 1..321: mol_type = other DNA; note = 2297.167.064 (Fel d1 MAb18)_cAb10206
gacatccagatgactcagtctccagcctccctatctgcatctgtgggagaaactgtcaccatcacatgtcgagcaagtgggaatattcacaattatttagcatggtttcagcagagacagggaaaatctcctcagctcctggtctataatgcagaaaccttagcagatggtgtgccatcaaggttcagtggcagtggatcaggaacacaatattctctcaagatcagcagcctgcagcctgaggactttgggagttatttctgtcaacatttttggagtagacctccgacgttcggtggaggcaccaagctggaaatcaag

SEQ ID NO: 39 - DNA, 321 bp, Mus musculus
  source 1..321: mol_type = other DNA; note = 2297.179.089 (Fel d1 MAb20)_cAb10207
aacattgtaatgacccaatctcccaaatccatgtccatgtcagtaggagagagggtcaccttgagctgcaaggccagtgagaatgtgggtagttatgtattctggtatcaacagaaaccagaccagtctcctaaactgctgatatacggggcatccaaccggtacactggggtccccgatcgcttcacaggcagtagatctgcaacagatttcactctgaccatcagcaatgtgcaggctgaagaccttgcagattatcactgtggacagagttacagctatccgctcacgttcggtgctgggaccaagctggagctgaaa

SEQ ID NO: 40 - DNA, 87 bp, synthetic construct
  source 1..87: mol_type = other DNA; note = 2297.179.089 (Fel d1 MAb20)_cAb10207
gccaaaacgacacccccatctgtctatccactggcccctggatctgctgcccaaactaactccatggtgaccctgggatgcctggtc

SEQ ID NO: 41 - DNA, 321 bp, Mus musculus
  source 1..321: mol_type = other DNA; note = 2291.014.055.067 (fel d1 MAb2)_cAb10204
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 42 - protein, 5 aa, synthetic construct
  source 1..5: note = 2291.014.055.067 (fel d1 MAb2)_cAb10204
SYDMS

SEQ ID NO: 43 - protein, 5 aa, synthetic construct
  source 1..5: note = 2293.254.030 (Fel d1 MAb9)_cAb10205
SYWMY

SEQ ID NO: 44 - protein, 5 aa, synthetic construct
  source 1..5: note = 2297.167.064 (Fel d1 MAb18)_cAb10206
SSYIS

SEQ ID NO: 45 - protein, 5 aa, synthetic construct
  source 1..5: note = 2297.179.089 (Fel d1 MAb20)_cAb10207
SYWIN

SEQ ID NO: 46 - protein, 17 aa, synthetic construct
  source 1..17: note = 2291.014.055.067 (fel d1 MAb2)_cAb10204
TISSGGTYTYYPDSLKG

SEQ ID NO: 47 - protein, 17 aa, synthetic construct
  source 1..17: note = 2293.254.030 (Fel d1 MAb9)_cAb10205
EINPSNGRTNYNEKFKT

SEQ ID NO: 48 - protein, 17 aa, synthetic construct
  source 1..17: note = 2297.167.064 (Fel d1 MAb18)_cAb10206
WIYAGTGGTTYNQRFTG

SEQ ID NO: 49 - protein, 17 aa, synthetic construct
  source 1..17: note = 2297.179.089 (Fel d1 MAb20)_cAb10207
NIYPSDSYTNYNQKFKD

SEQ ID NO: 50 - protein, 9 aa, synthetic construct
  source 1..9: note = 2291.014.055.067 (fel d1 MAb2)_cAb10204
LLRTYAMDY

SEQ ID NO: 51 - protein, 10 aa, synthetic construct
  source 1..10: note = 2293.254.030 (Fel d1 MAb9)_cAb10205
RATMIGGFAY

SEQ ID NO: 52 - protein, 11 aa, synthetic construct
  source 1..11: note = 2297.167.064 (Fel d1 MAb18)_cAb10206
HYGNPPAWFAY

SEQ ID NO: 53 - protein, 12 aa, synthetic construct
  source 1..12: note = 2297.179.089 (Fel d1 MAb20)_cAb10207
RGNYGNLYYFDY

SEQ ID NO: 54 - protein, 11 aa, synthetic construct
  source 1..11: note = 2291.014.055.067 (fel d1 MAb2)_cAb10204
HASQNIIVWLN

SEQ ID NO: 55 - protein, 17 aa, synthetic construct
  source 1..17: note = 2293.254.030 (Fel d1 MAb9)_cAb10205
KSSQSLLNSANQKNYLT

SEQ ID NO: 56 - protein, 11 aa, synthetic construct
  source 1..11: note = 2297.167.064 (Fel d1 MAb18)_cAb10206
RASGNIHNYLA

SEQ ID NO: 57 - protein, 11 aa, synthetic construct
  source 1..11: note = 2297.179.089 (Fel d1 MAb20)_cAb10207
KASENVGSYVF

SEQ ID NO: 58 - protein, 7 aa, synthetic construct
  source 1..7: note = 2291.014.055.067 (fel d1 MAb2)_cAb10204
KASNLHT

SEQ ID NO: 59 - protein, 7 aa, synthetic construct
  source 1..7: note = 2293.254.030 (Fel d1 MAb9)_cAb10205
WASTRES

SEQ ID NO: 60 - protein, 7 aa, synthetic construct
  source 1..7: note = 2297.167.064 (Fel d1 MAb18)_cAb10206
NAETLAD

SEQ ID NO: 61 - protein, 7 aa, synthetic construct
  source 1..7: note = 2297.179.089 (Fel d1 MAb20)_cAb10207
GASNRYT

SEQ ID NO: 62 - protein, 10 aa, synthetic construct
  source 1..10: note = 2291.014.055.067 (fel d1 MAb2)_cAb10204
QQGQSYHTWT

SEQ ID NO: 63 - protein, 9 aa, synthetic construct
  source 1..9: note = 2293.254.030 (Fel d1 MAb9)_cAb10205
QNDYSFPFT

SEQ ID NO: 64 - protein, 9 aa, synthetic construct
  source 1..9: note = 2297.167.064 (Fel d1 MAb18)_cAb10206
QHFWSRPPT

SEQ ID NO: 65 - protein, 9 aa, synthetic construct
  source 1..9: note = 2297.179.089 (Fel d1 MAb20)_cAb10207
GQSYSYPLT

SEQ ID NO: 66 - DNA, 972 bp, Mus musculus
  source 1..972: mol_type = other DNA; note = 2291.014.055.067 (fel d1 MAb2)_cAb10204
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 67 - protein, 442 aa, synthetic construct
  source 1..442: note = 2291.014.055.067 (fel d1 MAb2)_cAb10204
EVKLMESGGGLVKPGGSLKLSCAASGFTFSSYDMSWVRQTPEKRLEWVATISSGGTYTYYPDSLKGRFTISRDNARNTLYLQMSSLRSEDTALFYCARLLRTYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 68 - protein, 443 aa, synthetic construct
  source 1..443: note = 2293.254.030 (Fel d1 MAb9)_cAb10205
QVQLQQPGAELVKPGASVKLSCKASGYTFTSYWMYWVKQRPGQGLEWIGEINPSNGRTNYNEKFKTKATLTVDKSSSTAYMQLSSLTSEDSAVYYCARRATMIGGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 69 - protein, 444 aa, synthetic construct
  source 1..444: note = 2297.167.064 (Fel d1 MAb18)_cAb10206
QGQMQQSGAELVKPGASVKLSCKTSGFTFSSSYISWLKQKPGQSLEWIAWIYAGTGGTTYNQRFTGKAQLTVDSSSSTAYMHFSSLTTDDSAIYYCARHYGNPPAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 445 aa, synthetic construct
  source 1..445: note = 2297.179.089 (Fel d1 MAb20)_cAb10207
QVQLQQPGTELVRPGASVKLSCKASGYTFTSYWINWVKQRPGQGLEWIGNIYPSDSYTNYNQKFKDKATLTVDKSSSTAYMQLSSPTSDDSAVYYCTRRGNYGNLYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 71 - protein, 147 aa, synthetic construct
  source 1..147: note = 2297.179.089 (Fel d1 MAb20-1)_cAb10207
QIQLVQSGPELRKPGETVRISCKASGYTFTTYGMDWVKQAPGKVLKWVGWINTNTGEPTYAEEFKGRFAFSLETSASTAYFQINNLKNEDTATYFCARKWLRRAMDYWGQGTSVTVSSAKTTPPSVYPLAPGSAAQTNSMVTLGCLV

SEQ ID NO: 72 - protein, 215 aa, synthetic construct
  source 1..215: note = 2291.014.055.067 (fel d1 MAb2)_cAb10204
DIQMNQSPSSLSASLGDTITITCHASQNIIVWLNWYQQKPGNIPKLLIYKASNLHTGVPSRFSGSGSGTGFTLTISSLQPEDIATYYCQQGQSYHTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 73 - protein, 220 aa, synthetic construct
  source 1..220: note = 2293.254.030 (Fel d1 MAb9)_cAb10205
DIVMTQSPSSLTVTTGEKVTMNCKSSQSLLNSANQKNYLTWHQQKPGQPPKLLIYWASTRESGVPDRFTGSGSGTDFTLTISSVQAEDLAVYYCQNDYSFPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 74 - protein, 214 aa, synthetic construct
  source 1..214: note = 2297.167.064 (Fel d1 MAb18)_cAb10206
DIQMTQSPASLSASVGETVTITCRASGNIHNYLAWFQQRQGKSPQLLVYNAETLADGVPSRFSGSGSGTQYSLKISSLQPEDFGSYFCQHFWSRPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 75 - protein, 214 aa, synthetic construct
  source 1..214: note = 2297.179.089 (Fel d1 MAb20)_cAb10207
NIVMTQSPKSMSMSVGERVTLSCKASENVGSYVFWYQQKPDQSPKLLIYGASNRYTGVPDRFTGSRSATDFTLTISNVQAEDLADYHCGQSYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0296993 A1
(43) Pub. Date: Sep. 25, 2025

(54) ANTI-FEL D 1 ANTIBODIES AND ANTIGEN BINDING FRAGMENTS THEREOF AND USES THEREOF

(71) Applicant: R-CAT Technologies Inc., Malden, MA (US)

(72) Inventor: Kasey Lynne Jackson, Malden, MA (US)

(73) Assignee: R-CAT Technologies Inc., Malden, MA (US)

(21) Appl. No.: 19/061,948

(22) Filed: Feb. 24, 2025

Related U.S. Application Data
(60) Provisional application No. 63/560,069, filed on Mar. 1, 2024.

(51) Int. Cl. C07K 16/18; G01N 33/543; G01N 33/68

(52) U.S. Cl. CPC C07K 16/18; G01N 33/543; G01N 33/68; C07K 2317/565; G01N 2333/46

(57) ABSTRACT

The present disclosure generally relates to antibodies and antigen-binding fragments thereof that bind to Fel d 1 and uses thereof.

### CROSS-REFERENCE TO RELATED APPLICATIONS

This application claims the benefit of priority under 35 U.S.C. § 119(e) of U.S. Provisional Patent Application No. 63/560,069, filed Mar. 1, 2024, the entire contents of which is incorporated herein by reference in its entirety.

The instant application contains a Sequence Listing which has been submitted electronically in XMIL format and is hereby incorporated by reference in its entirety. Said XML copy, created on May 28, 2025, is named SequenceListing_127896-0104.xml and is 88,000 bytes in size.

### BACKGROUND

The following description of the background of the present technology is provided simply as an aid in understanding the present technology and is not admitted to describe or constitute prior art to the present technology.

The domestic cat, Felis domesticus (Fel d) or Felis catus, is one of the most frequently encountered pets. However, domestic cats are also a major source of indoor allergens and are placed second only to dust mites for their involvement in the incidence of allergic respiratory diseases. Hence, allergies to cats are widespread, with a prevalence of 10/6-30% in the Western population. A total number of ten Fel d allergens that are recognized by human IgEs have been identified, one of them being Fel d 1. Fel d 1, a uteroglobulin-like protein, is considered to be the major cat allergen. Fel d 1 is understood to be shed from the cat to the environment through, for example, airborne dander and, if inhaled by humans, may result in sensitization and induction of cat allergy. All cats produce Fel d 1; however, cats can produce varying levels of Fel d 1 depending on, for example, neuter status, sex, and/or genetics, and not all cats shed Fel d 1 into the air at the same rate.

At least because of the importance of Fel d 1 in allergic reactions to cats, there remains a need for efficient and sensitive technologies for detection and/or quantification of Fel d 1, in particular, in samples from domestic cats or environmental samples.

### SUMMARY OF THE DISCLOSURE

The present disclosure provides, among other things, antibodies and antigen-binding fragments thereof that bind (e.g., specifically bind) to Fel d 1. Such antibodies and antigen-binding fragments thereof are useful in, for example, the detection and quantification of Fel d 1 and can be incorporated into various detection methods, such as, lateral flow assays.

In one aspect, the present disclosure provides antibodies or antigen-binding fragments thereof (e.g., isolated antibodies or antigen-binding fragments thereof) that bind to Fel d 1, wherein the antibody or the antigen-binding fragment thereof comprises: (a) a heavy chain complementary determining region 1 (CDRH1) comprising the amino acid sequence of any one of SEQ ID NOs: 42-45, a heavy chain complementary determining region 2 (CDRH2) comprising the amino acid sequence of any one of SEQ ID NOs: 46-49, and a heavy chain complementary determining region 3 (CDRH3) comprising the amino acid sequence of any one of SEQ ID NOs: 50-53; and (b) a light chain complementary determining region 1 (CDRL1) comprising the amino acid sequence of any one of SEQ ID NOs: 54-57, a light chain complementary determining region 2 (CDRL2) comprising the amino acid sequence of any one of SEQ ID NOs: 58-61, and a light chain complementary determining region 3 (CDRL3) comprising the amino acid sequence of any one of SEQ ID NOs: 62-65.

In some embodiments, the isolated antibody or antigen-binding fragment there of comprises: (a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; (b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; (c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or (d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the antibody or antigen-binding fragment thereof comprises a heavy chain variable domain sequence (VH) and a light chain variable domain sequence (VL) comprising at least 96%, at least 97%, at least 98%, or at least 99% identity to an amino acid sequence selected from: (a) the amino acid sequence of SEQ ID NO: 10 and the amino acid sequence of SEQ ID NO: 15; (b) the amino acid sequence of SEQ ID NO: 11 and the amino acid sequence of SEQ ID NO: 16; (c) the amino acid sequence of SEQ ID NO: 12 and the amino acid sequence of SEQ ID NO: 17; or (d) the amino acid sequence of SEQ ID NO: 13 and the amino acid sequence of SEQ ID NO: 18.

In some embodiments, the antibody or antigen-binding fragment thereof comprises: (a) the amino acid sequence of SEQ ID NO: 10 and the amino acid sequence of SEQ ID NO: 15; (b) the amino acid sequence of SEQ ID NO: 11 and the amino acid sequence of SEQ ID NO: 16; (c) the amino acid sequence of SEQ ID NO: 12 and the amino acid sequence of SEQ ID NO: 17; or (d) the amino acid sequence of SEQ ID NO: 13 and the amino acid sequence of SEQ ID NO: 18.

In some embodiments, the antibody or antigen-binding fragment thereof is a monoclonal antibody.

In some embodiments, the antigen-binding fragment thereof is a recombinant single chain fragment variable or single chain variable domain fragment (scFv) antibody, a Fab fragment, a F(ab′)2 fragment, or a variable domain fragment (Fv fragment). In some embodiments, the antigen-binding fragment thereof is a recombinant antibody variable domain fragment (Fv fragment) and/or wherein the antigen-binding fragment thereof comprises the amino acid sequence of one or more of SEQ ID NOs: 10-18. In some embodiments, the antigen-binding fragment thereof further comprises a linker domain and wherein the linker domain is operably linked to one or both of the heavy chain variable domain and the light chain variable domain.

In some embodiments, the antibody is an IgG antibody.

In some embodiments, the antibody or antigen-binding fragment thereof is conjugated to a label.

In some embodiments, the antibody or antigen-binding fragment thereof further comprises a label.

In one aspect, the present disclosure provides an isolated nucleic acid comprising a nucleotide sequence encoding an antibody or antigen-binding fragment thereof that binds to Fel d 1, wherein the antibody or antigen-binding fragment thereof comprises: (a) a heavy chain complementary determining region 1 (CDRH1) comprising the amino acid sequence of any one of SEQ ID NOs: 42-45, a heavy chain complementary determining region 2 (CDRH2) comprising the amino acid sequence of any one of SEQ ID NOs: 46-49, and a heavy chain complementary determining region 3 (CDRH3) comprising the amino acid sequence of any one of SEQ ID NOs: 50-53; and (b) a light chain complementary determining region 1 (CDRL1) comprising the amino acid sequence of any one of SEQ ID NOs: 54-57, a light chain complementary determining region 2 (CDRL2) comprising the amino acid sequence of any one of SEQ ID NOs: 58-61, and a light chain complementary determining region 3 (CDRL3) comprising the amino acid sequence of any one of SEQ ID NOs: 62-65.

In some embodiments, the isolated nucleic acid comprises a nucleotide sequence encoding an antibody or antigen-binding fragment thereof that binds to Fel d 1, wherein the antibody or antigen-binding fragment thereof comprises: (a) the amino acid sequence of SEQ ID NO: 10 and the amino acid sequence of SEQ ID NO: 15; (b) the amino acid sequence of SEQ ID NO: 11 and the amino acid sequence of SEQ ID NO: 16; (c) the amino acid sequence of SEQ ID NO: 12 and the amino acid sequence of SEQ ID NO: 17; or (d) the amino acid sequence of SEQ ID NO: 13 and the amino acid sequence of SEQ ID NO: 18.

In some embodiments, the present disclosure provides a vector comprising an isolated nucleic acid of the present disclosure.

In some embodiments, the present disclosure provides a composition comprising one or more antibody or antigen-binding fragments thereof of the present disclosure, an isolated nucleic acid of the present disclosure, or a vector of the present disclosure.

In one aspect, the present disclosure provides a method of detecting or quantifying Fel d 1 in a sample comprising: (a) contacting the sample with the antibody or antigen-binding fragment thereof of the present disclosure; and (b) detecting Fel d 1 in the sample by detecting the binding of the antibody or the antigen-binding fragment thereof to the Fel d 1 in the sample.

In some embodiments, the sample is a biological sample isolated from a feline. In some embodiments, the biological sample is isolated from one or more of the saliva, anal glands, urine, sebaceous glands, skin, and fur of a feline. In some embodiments, the feline is of the species Felis catus. In some embodiments, the sample is an environmental sample.

In some embodiments, the detection comprises enzyme-linked immunosorbent assay (ELISA), lateral flow assay, immunohistochemistry, immunofluorescence, or Western blot.

In one aspect, the present disclosure provides a method of detecting or quantifying Fel d 1 in a sample comprising: (a) contacting the sample with a first antibody or antigen-binding fragment thereof selected from an anti-Fel d 1 antibody or antigen-binding fragment thereof of the present disclosure, thereby producing a first complex comprising the Fel d 1 and the first antibody or antigen-binding fragment thereof; (b) contacting the first complex with a second antibody or antigen-binding fragment thereof selected from an anti-Fel d 1 antibody or antigen-binding fragment thereof of the present disclosure, thereby producing a second complex comprising the Fel d 1, the first antibody or antigen-binding fragment thereof, and the second antibody or antigen-binding fragment thereof; wherein either of the first antibody or antigen-binding fragment thereof or the second antibody or antigen-binding fragment thereof comprises a label capable of producing a signal; and (c) measuring the signal.

In some embodiments, the first antibody or antigen-binding fragment thereof and the second antibody or antigen-binding fragment thereof are from different species.

In some embodiments, the first antibody or antigen-binding fragment thereof and the second antibody or antigen-binding fragment thereof are from the same species.

In some embodiments, the first antibody or antigen-binding fragment thereof and the second antibody or antigen-binding fragment thereof are different isotypes or subclasses. In some embodiments, the different subclasses are selected from mouse IgG1, IgG2A, IgG2B, IgG2C, and IgG3.

In some embodiments, the first antibody or antigen-binding fragment thereof and the second antibody or antigen-binding fragment thereof comprise the same complementary determining regions (CDRs).

In some embodiments, the first antibody or antigen-binding fragment thereof and the second antibody or antigen-binding fragment thereof comprise different CDRs.

In some embodiments, the first antibody or antigen-binding fragment thereof, the second antibody or antigen-binding fragment thereof, or both is an IgG, optionally an IgG1.

In some embodiments, the first antibody or antigen-binding fragment thereof and the second antibody or antigen-binding fragment thereof are from different species.

In some embodiments, the label comprises colloidal gold, horseradish peroxidase (HRP), or a dye. In some embodiments, the dye comprises a fluorescent dye.

In some embodiments, the first antibody or antigen-binding fragment thereof comprises: (a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; (b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; (c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or (d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the second antibody or antigen-binding fragment thereof comprises: (a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; (b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; (c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or (d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In one aspect, the present disclosure provides a kit comprising one or more antibodies or antigen-binding fragments thereof of the present disclosure. In some embodiments, the kit further comprises a secondary antibody that binds to the second antibody or antigen-binding fragment thereof and which comprises a detectable label. In some embodiments, the detectable label is HRP or a fluorescent dye (e.g., a fluorophore). In some embodiments, the kit further comprises one or more reagents for detecting Fel d 1. In some embodiments, the one or more reagents comprise blocking buffer, a wash buffer, Fel d 1 polypeptide, and/or a positive control.

### BRIEF DESCRIPTION OF THE DRAWINGS

FIG. 1 shows the results of evaluating of test bleed 1 (test 1 sera) by mouse immune response to plate trapped Fel d 1 antigen ELISA.

FIG. 2 shows the results of evaluating test bleed 1 (test 1 sera) by mouse immune response to Fel d 1 biotin capture ELISA.

FIG. 3 shows results of evaluating test bleed 2 by mouse immune response to plate trapped Fel d 1 antigen ELISA.

FIG. 4 shows results of titration of Purified Fel d1 Monoclonal cell line antibodies against natural Fel d 1. Six monoclonal cell lines resulted from the five mice spleen fusions.

FIG. 5 provides a visual representation of chequerboard ELISA results.

FIG. 6A-6C provides results from sandwich ELISA standard curves. FIG. 6A shows the ELISA standard curve results for pairing MAb20 and conjugated mAb2 (Conj2). FIG. 6B shows the ELISA standard curves results for pairing MAb20 and conjugated mAb18 (Conj18) and MAb2 and conjugated mAb20 (Conj20). FIG. 6C shows the ELISA standard curve results for remaining mAb pairings.

FIG. 7 demonstrates results from initial testing of antibody pairings in different orientations using a range of Fel d 1 concentrations.

FIG. 8 shows a graph comparing pairings 1 (conjugated mAb2 and NC mAb20) and 2 (conjugated mAb20 and NC mAb2) at three different spray rates.

FIG. 9 provides dilution curves of pairing 1 (conjugated mAb2 and NC mAb20) and 2 (conjugated mAb20 and NC mAb2) across dilution 10 μg/ml-0.0003 μg/ml.

FIG. 10 provides an image of initial dry testing of pairing 1 (conjugated mAb2 and NC mAb20).

FIG. 11 provides an image of initial dry testing of pairing 2 (conjugated mAb20 and NC mAb2).

### DETAILED DESCRIPTION

The domestic cat, Felis domesticus (Fel d) or Felis catus, is one of the most frequently encountered pets. However, domestic cats are also a major source of indoor allergens and are placed second only to dust mites for their involvement in the incidence of allergic respiratory diseases. Hence, allergies to cats are widespread, with a prevalence of 10/6-30% in the Western population. A total number of ten Fel d allergens that are recognized by human IgEs have been identified, one of them being Fel d 1. Fel d 1, a uteroglobulin-like protein, is considered the major cat allergen. Fel d 1 is understood to be shed from the cat to the environment, for example, through airborne dander and, if inhaled by humans, may result in sensitization and induction of cat allergy.

All cats produce Fel d 1; however, cats can produce varying levels of Fel d 1 depending on, for example, neuter status, sex, and/or genetics and not all cats shed Fel d 1 into the air at the same rate. Thus, detecting and/or quantifying Fel d 1 in biological or environmental samples is important for people who suffer from cat allergies. For example, such technologies may assist them in selecting a cat that produces lower levels of Fel d 1 as a pet (e.g., and thereby potentially reducing their allergic response) and/or determining whether cat allergen, Fel d 1, is present in their environment (e.g., home, clothing). Accordingly, there remains a need for efficient and sensitive technologies for detection and/or quantification of Fel d 1, in particular, in samples from domestic cats or environmental samples.

The present disclosure provides, among other things, antibodies and antigen-binding fragments thereof that bind (e.g., specifically bind) to Fel d 1. Such antibodies and antigen-binding fragments thereof are useful in, for example, the detection and/or quantification of Fel d 1 and can be incorporated into various detection methods, such as, lateral flow assays. Additionally, antibodies and antigen-binding fragments thereof of the present disclosure can be felinized (or undergo “felinization”) and used, e.g., for reducing (e.g., compared to an appropriate reference standard) the level of active Fel d 1 in or on a subject (e.g., a cat).

It is to be appreciated that certain aspects, modes, embodiments, variations, and features of the present technologies are described below in various levels of detail in order to provide a substantial understanding of the present technologies.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary.

It is also to be understood that the terminology used herein is for the purpose of describing particular embodiments only, and is not intended to be limiting.

### Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the singular forms “a”, “an”, and “the” include the singular and plural referents unless the context clearly dictates otherwise. For example, the term “a cell” includes a single cell as well as a plurality of cells, including mixtures thereof, and means one cell or more than one cell.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) (f) 20%, 15%, 10%, 5%, 3%, 2%, or 1%. Preferably ±5%, more preferably ±1%, and still more preferably ±0.1% from the specified value, as such variations are appropriate to perform the disclosed methods.

As used herein, the term “affinity” refers to a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference]. In some embodiments, affinity may be assessed relative to a contemporaneous reference; in some embodiments, affinity may be assessed relative to a historical reference. Typically, when affinity is assessed relative to a reference, it is assessed under comparable conditions.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “Antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response.

As used herein, the term “antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, variable domain fragment (Fv), Fab and F(ab′)2, as well as single chain antibodies (scFv) and felinized antibodies. In some embodiments, antibody refers to such assemblies (e.g., intact antibody molecules, immunoadhesins, or variants thereof) which have significant known specific immunoreactive activity to an antigen of interest (e.g., Fel d 1). Antibodies and immunoglobulins comprise light and heavy chains, with or without an interchain covalent linkage between them. Basic immunoglobulin structures in vertebrate systems are relatively well understood.

In some embodiments, an antibody is a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules. In one embodiment, the antibody or antibody molecule comprises, e.g., consists of, an antibody fragment or an antigen-binding fragment thereof.

The term “antibody fragment” or “antigen-binding fragment thereof” refers to a portion of an intact antibody and refers to the antigenic determining variable regions of an intact antibody. In some embodiments, the term “antibody fragment” or “antigen-binding fragment thereof” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen (e.g., Fel d 1). Examples of antibody fragments include, but are not limited to, Fab, Fab′, F(ab′)2, Fv fragments, scFv antibody fragments, linear antibodies, single domain antibodies such as sdAb (either VL or VH), camelid VHH domains, and multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment thereof can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen-binding fragments thereof can also be grafted into scaffolds based on polypeptides such as a fibronectin type III (Fn3) (see U.S. Pat. No. 6,703,199, which describes fibronectin polypeptide minibodies). “Fab” means a monovalent antigen-binding fragment of an immunoglobulin that is composed of the light chain and part of the heavy chain. F(ab′)2 means a bivalent antigen-binding fragment of an immunoglobulin that contains both light chains and part of both heavy chains.

As used herein, the term “antibody heavy chain” refers to the larger of the two types of polypeptide chains present in all antibody molecules in their naturally occurring conformations.

As used herein, the term “antibody light chain” refers to the smaller of the two types of polypeptide chains present in all antibody molecules in their naturally occurring conformations. Kappa (K) and lambda (λ) light chains refer to the two major antibody light chain isotypes.

As used herein, the term “camelid VHH domain” refers to Camelidae (e.g., camels, dromedaries, and llamas) heavy-chain-only antibodies. These heavy-chain-only antibodies possess a single variable domain, termed VHH, that is responsible for antigen recognition.

As used herein, the term “felinized” or “felinization” refers to a method for transferring non-feline antigen-binding information from a donor antibody to a less immunogenic feline antibody acceptor to generate treatments useful as therapeutics in cats. Felinized antibodies are feline antibody sequences in which hypervariable region residues of the recipient are replaced by hypervariable region residues from a non-feline species (donor antibody) such as such as mouse, rat, rabbit, cat, dogs, goat, chicken, bovine, horse, llama, camel, dromedaries, sharks, non-human primates, human, humanized, recombinant sequence, or an engineered sequence having the desired properties, e.g., specificity, affinity, capacity. Furthermore, felinized antibodies may include residues that are not found in the recipient antibody or in the donor antibody. These modifications are made to further refine antibody performance. The modifications to the hypervariable regions and/or the framework regions, as described herein, are determined for each separately engineered speciated (felinized) antibody based on experimentation known to those in the art yet cannot be predicted prior to said experimentation. The felinized antibody may comprise a complete, or at least a portion of an immunoglobulin constant region (Fc), typically that of a feline immunoglobulin.

As used herein, the term “felinized antibody” refers to feline forms of non-feline (e.g., mouse) antibodies, and are chimeric immunoglobulins, immunoglobulin chains or fragments thereof (such as Fv, Fab, Fab′, F(ab′)2 or other antigen-binding subsequences of antibodies), which contain minimal sequence derived from non-feline immunoglobulin. For the most part, felinized antibodies are feline immunoglobulins (recipient antibody) in which residues from a complementary-determining region (CDR) of the recipient are replaced by residues from a CDR of a non-feline species (donor antibody) such as mouse, rat or rabbit having the desired properties such as specificity, affinity, and/or capacity. In some instances, Fv framework region (FR) residues of the feline immunoglobulin are replaced by corresponding non-feline residues. Furthermore, felinized antibodies can comprise residues which are found neither in the recipient antibody nor in the imported CDR or framework sequences. These modifications are made to further refine and optimize antibody performance. In general, the felinized antibody will comprise substantially all of at least one, and typically two, variable domains, in which all or substantially all of the CDR regions correspond to those of a non-feline immunoglobulin and all or substantially all of the FR regions are those of a feline immunoglobulin sequence. The felinized antibody optimally also will comprise at least a portion of an immunoglobulin constant region (Fc), typically that of a feline immunoglobulin.

As used herein, the term “Fv fragment” or “variable domain fragment” refers to a VH domain and a VL domain of an antibody specifically binding to an antigen, both domains forming together a Fv fragment. In some embodiment, Fv fragments means an antibody fragment comprising the VH and VL domains of an antibody, wherein these domains are present in a single polypeptide chain. Generally, the Fv fragment polypeptide further comprises a polypeptide linker between the VH and VL domains polypeptide that enables the scFv to form.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “synthetic antibody” means an antibody, which is generated using recombinant DNA technology, such as, for example, an antibody expressed by a bacteriophage. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “binding”, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently, electrostatically, or otherwise associated with a carrier entity and/or in a biological system or cell). Binding between two entities may be considered “specific” if, under the conditions assessed, the relevant entities are more likely to associate with one another than with other available binding partners.

As used herein, the term “complementarity determining region” or “CDR” refers to the sequences of amino acids within antibody variable regions which confer antigen specificity and binding affinity. For example, in general, there are three CDRs in each heavy chain variable region (e.g., CDRH1, CDRH2, and CDRH3) and three CDRs in each light chain variable region (CDRL1, CDRL2, and CDRL3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof, North et al., J. Molecular Biology, 406 (2): 228-256 (2011) and; Lefranc et al. Nucl. Acids Res. 27:209-212 (1999) or Ruiz et al. Nucl. Acids Res. 28:219-221 (2000)) (IMGT numbering scheme).

Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). Under the IMGT numbering scheme, in some embodiments, the CDR amino acids in the VH are numbered 27-38 (HCDR1), 56-65 (HCDR2), and 105-120 (HCDR3); and the CDR amino acid residues in the VL are numbered 27-38 (LCDR1), 56-65 (LCDR2), and 105-120 (LCDR3).

In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g, lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g, tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

As used herein, the term “recombinant host cell,” “recombinant cell,” “engineered host cell,” or “engineered cell,” means a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” or “cell” as used herein. Host cells include progeny of a single host cell, and the progeny may not necessarily be completely identical (in morphology or in total DNA complement) to the original parent cell due to natural, accidental, or deliberate mutation and/or change. A host cell includes a cell transfected or infected in vivo or in vitro with a recombinant vector, an expression vector, or a nucleic acid encoding an antibody of the present disclosure. A host cell, which comprises a recombinant vector, expression vector, or a nucleic acid encoding an antibody disclosed herein, may also be referred to as a “recombinant host cell,” “engineered host cell,” or “engineered cell”.

As used herein, the term “host cell” refers to a cell, which may be used in a process for purifying an immunogenic protein or recombinant antibody in accordance with the present disclosure. Such host cell expresses the protein of interest (the antibody disclosed herein). A host cell may also be referred to as a protein-expressing cell. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell, according to the present disclosure, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli. Erwinia sp., Klebsellia sp., Lactobacillus sp. ox Bacillus subtilis. In some embodiments, the host cell is a yeast cell. In that embodiment, the yeast host cell is selected from the group consisting of Saccharomyces cerevisiae. Hansenula polymorpha, and Pichia pastoris.

“Effective amount” or “therapeutically effective amount” as used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal (e.g., cat), causes a detectable level of reduction in the amount of active Fel d 1 compared to that detected in the absence of the composition of the invention. The level of active Fel d 1 can be readily assessed by methods described herein and any appropriate art-recognized methods. The skilled artisan would understand that the amount of the composition administered herein varies and can be readily determined based on a number of factors such as the age and health and physical condition of the mammal being administered such a composition (e.g., a cat), the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

In some embodiments, an “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the agent (e.g., antibody, antigen-binding fragment thereof, pharmaceutical composition), the route of administration, etc. It is understood, however, that specific dose levels of the agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10. In one embodiment, the flexible polypeptide linkers include, but are not limited to, (Gly4 Ser)4 or (Gly4 Ser)3. In another embodiment, the linkers include multiple repeats of (Gly2Ser), (GlySer) or (Gly3Ser).

As used herein the term, “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

As used herein, the term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “pharmaceutical composition” refers to an active agent (e.g., anti-Fel d 1 antibody or antigen-binding fragment thereof of the present disclosure), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for a particular route of administration, e.g., as described herein.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “reference standard” describes a standard or control relative to which a comparison is performed. For example, in some embodiments, an agent (e.g., an antibody or antigen-binding fragment thereof described herein), animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “sample” and “biological sample” and “environmental sample” are used interchangeably, referring to sample material. Biological samples may include tissues, cells, protein or membrane extracts of cells, and biological fluids (e.g., ascites fluid or cerebrospinal fluid (CSF)) isolated from a subject (e.g., a cat), as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair or fur, buccal, skin, serum, plasma, CSF, semen, prostate fluid, seminal fluid, urine, feces, sweat, saliva, sputum, mucus, glands (e.g., anal glands, sebaceous glands) bone marrow, lymph, and tears. Environmental samples include soil, foliage or any plant tissue or surface or other sample suspected of harboring an allergen (e.g., Fel d 1). In addition, the environmental sample can include industrial samples, such as those isolated from surfaces and the environment.

As used herein, the term “sequence identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. Methods to determine “sequence identity” by conducting sequence alignment are readily known in the art and their use is well within the level of one of ordinary skill in the art.

As used herein, the term “single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, Science 242:423-442 (1988); Huston et al., Proc. Natl. Acad. Sci. USA 85:5879-5883 (1988); Ward et al., Nature 334:54454 (1989); Skerra et al., Science 242: 1038-1041 (1988). [0218]

As used herein, the term “specifically binds,” with respect to an antibody or antigen-binding fragment thereof, means an antibody or binding fragment thereof (e.g., Fv fragment or scFv) recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species (e.g., an antigen, e.g., Fel d 1), to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the terms “subject” and “patient” are used interchangeably. As used herein, a subject can be a mammal. In certain embodiments, the term “subject,” as used herein, refers to a vertebrate, such as a mammal of the Felidae family. In certain exemplary embodiments, a subject is a cat (e.g., a domestic cat).

As used herein, the term “therapy” refers to any protocol, method and/or agent (e.g., antibody, antigen-binding fragment thereof, pharmaceutical composition) that can be used in the prevention, management, treatment and/or amelioration of an allergy.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule.

To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

As used herein, the term “vector” is a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

### Antibodies and Antigen-Binding Fragments Thereof

One aspect of the present disclosure provides an antibody or antigen-binding fragment thereof (e.g., an isolated antibody or antigen-binding fragment thereof) that specifically binds to Fel d 1 or a variant thereof.

Of the ten cat allergens identified to date, Fel d 1 is the most important allergen to which most individuals with cat allergies are sensitized. Fel d 1 belongs to the family of secretoglobins or secretory globins and is a 35-39 kDa glycoprotein produced primarily by the sebaceous glands in cats. Fel d 1 is a thermostable protein found in, for example, the saliva, anal glands, sebaceous glands, skin and fur of cats. Fel d 1 consists of two identical heterodimers, each of 18-19 kDa, linked noncovalently and eventually forming a tetramer. Each dimer consists of two polypeptide chains, chain 1 and chain 2, covalently linked by three disulfide bridges and encoded by two different genes (Ch1 and Ch2).

While the exact biological role of Fel d 1 is yet to be conclusively determined, it is understood to have numerous functions, such as skin protection or transport of lipids, especially steroids, hormones, and pheromones. Fel d 1 can be transferred to the hair/fur when cats groom and is subsequently shed with hair/fur and dander. Without wishing to be bound by any one theory, it is understood that because of the small size of Fel d 1 and its molecular structure, Fel d 1 can be airborne for long periods of time as well as adhere to surfaces, such as fabrics and indoor furniture, thereby increasing the probability of exposure. Upon exposure in sensitized individuals, the unbound form of Fel d 1, termed active Fel d 1, binds IgE and can lead to mast cell degranulation, thus initiating the allergic response cascade.

In some embodiments, an antibody or antigen-binding fragment thereof disclosed herein binds (e.g., specifically binds) to Fel d1 or a variant thereof.

In some embodiments, the antibody or antigen-binding fragment thereof comprises: (a) a heavy chain complementary determining region 1 (CDRH1) comprising the amino acid sequence of any one of SEQ ID NOs: 42-45, a heavy chain complementary determining region 2 (CDRH2) comprising the amino acid sequence of any one of SEQ ID NOs: 46-49, and a heavy chain complementary determining region 3 (CDRH3) comprising the amino acid sequence of any one of SEQ ID NOs: 50-53; and (b) a light chain complementary determining region 1 (CDRL1) comprising the amino acid sequence of any one of SEQ ID NOs: 54-57, a light chain complementary determining region 2 (CDRL2) comprising the amino acid sequence of any one of SEQ ID NOs: 58-61, and a light chain complementary determining region 3 (CDRL3) comprising the amino acid sequence of any one of SEQ ID NOs: 62-65.

In some embodiments, the antibody or antigen-binding fragment thereof comprises a heavy chain complementary determining region 1 (CDRH1) comprising, or consisting essentially of, or consisting of the amino acid sequence of SEQ ID NO: 42, a CDRH2 comprising, or consisting essentially of, or consisting of the amino acid sequence of SEQ ID NO: 46, and a CDRH3 comprising, or consisting essentially of, or consisting of the amino acid sequence of SEQ ID NO: 50, and a light chain complementary determining region 1 (CDRL1) comprising, or consisting essentially of, or consisting of the amino acid sequence of SEQ ID NO: 54, a CDRL2 comprising, or consisting essentially of, or consisting of the amino acid sequence of SEQ ID NO: 58, and a CDRL3 comprising, or consisting essentially of, or consisting of the amino acid sequence of SEQ ID NO: 62.

In some embodiments, the antibody or antigen-binding fragment thereof comprises a heavy chain complementary determining region 1 (CDRH1) comprising, or consisting essentially of, or consisting of the amino acid sequence of SEQ ID NO: 43, a CDRH2 comprising, or consisting essentially of, or consisting of the amino acid sequence of SEQ ID NO: 47, and a CDRH3 comprising, or consisting essentially of, or consisting of the amino acid sequence of SEQ ID NO: 51, and a light chain complementary determining region 1 (CDRL1) comprising, or consisting essentially of, or consisting of the amino acid sequence of SEQ ID NO: 55, a CDRL2 comprising, or consisting essentially of, or consisting of the amino acid sequence of SEQ ID NO: 59, and a CDRL3 comprising, or consisting essentially of, or consisting of the amino acid sequence of SEQ ID NO: 63.

In some embodiments, the antibody or antigen-binding fragment thereof comprises a heavy chain complementary determining region 1 (CDRH1) comprising, or consisting essentially of, or consisting of the amino acid sequence of SEQ ID NO: 44, a CDRH2 comprising, or consisting essentially of, or consisting of the amino acid sequence of SEQ ID NO: 48, and a CDRH3 comprising, or consisting essentially of, or consisting of the amino acid sequence of SEQ ID NO: 52, and a light chain complementary determining region 1 (CDRL1) comprising, or consisting essentially of, or consisting of the amino acid sequence of SEQ ID NO: 56, a CDRL2 comprising, or consisting essentially of, or consisting of the amino acid sequence of SEQ ID NO: 60, and a CDRL3 comprising, or consisting essentially of, or consisting of the amino acid sequence of SEQ ID NO: 64.

In some embodiments, the antibody or antigen-binding fragment thereof comprises a heavy chain complementary determining region 1 (CDRH1) comprising, or consisting essentially of, or consisting of the amino acid sequence of SEQ ID NO: 45, a CDRH2 comprising, or consisting essentially of, or consisting of the amino acid sequence of SEQ ID NO: 49, and a CDRH3 comprising, or consisting essentially of, or consisting of the amino acid sequence of SEQ ID NO: 53, and a light chain complementary determining region 1 (CDRL1) comprising, or consisting essentially of, or consisting of the amino acid sequence of SEQ ID NO: 57, a CDRL2 comprising, or consisting essentially of, or consisting of the amino acid sequence of SEQ ID NO: 61, and a CDRL3 comprising, or consisting essentially of, or consisting of the amino acid sequence of SEQ ID NO: 65.

In some embodiments, the CDRs of the heavy chain variable domain or the light chain variable domain of an antibody or antigen-binding fragment thereof disclosed herein comprises the amino acid sequences disclosed in Table 13.

In some embodiments, an antibody or antigen-binding fragment thereof of the present disclosures comprises: a heavy chain variable domain sequence (VH) having at least 70%, 80%, 90%, 95%, 96%, 97%, 98%, or 99% identity to the amino acid sequence of any one of: SEQ ID NOs: 10-14; and/or a light chain variable domain sequences (VL) having at least 70%, 80%, 90%, 95%, 96%, 97%, 98%, or 99% identity to the amino acid sequence of any one of: SEQ ID NOs: 15-18. In some embodiments, the CDRs of the heavy chain variable domain sequence or the light chain variable domain sequence are 100% identical to a CDR disclosed in Table 13.

In some embodiments, an antibody or antigen-binding fragment thereof disclosed herein comprises: a heavy chain variable domain sequence having at least 70%, 80%, 90%, 95%, 96%, 97%, 98%, or 99% identity to the amino acid sequence disclosed in Table 9 or Table 13; and/or a light chain variable domain sequences having at least 70%, 80%, 90%, 95%, 96%, 97%, 98%, or 99% identity to the amino acid sequence disclosed in Table 10 or Table 13. In some embodiments, the CDRs of the heavy chain variable domain sequence or the light chain variable domain sequences are 100% identical to a CDR disclosed in Table 13.

In some embodiments, an antibody or antigen-binding fragment disclosed herein comprises, or consists essentially of, or consists of a heavy chain variable domain sequence and a light chain variable domain sequence having at least 96%, at least 97%, at least 98%, at least 99%, or 100% identity to an amino acid sequence selected from: the amino acid sequence of SEQ ID NO: 10 and the amino acid sequence of SEQ ID NO: 15; the amino acid sequence of SEQ ID NO: 11 and the amino acid sequence of SEQ ID NO: 16; the amino acid sequence of SEQ ID NO: 12 and the amino acid sequence of SEQ ID NO: 17; or the amino acid sequence of SEQ ID NO: 13 and the amino acid sequence of SEQ ID NO: 18.

In some embodiments, an antibody or antigen-binding fragment disclosed herein comprises, or consists essentially of, or consists of: the amino acid sequence of SEQ ID NO: 10 and the amino acid sequence of SEQ ID NO: 15; the amino acid sequence of SEQ ID NO: 11 and the amino acid sequence of SEQ ID NO: 16; the amino acid sequence of SEQ ID NO: 12 and the amino acid sequence of SEQ ID NO: 17; or the amino acid sequence of SEQ ID NO: 13 and the amino acid sequence of SEQ ID NO: 18.

In some embodiments, an antibody of the present disclosure comprises, consists of, or consists essentially of a heavy chain having at least 96%, at least 97%, at least 98%, at least 99%, or 100% identity to the amino acid sequence of any one of SEQ ID NOs: 67-71 and a light chain having at least 96%, at least 97%, at least 98%, at least 99%, or 100% identity to the amino acid sequence of any one of SEQ ID NOs: 72-76.

In some embodiments, an antibody of the present disclosure comprises, consists of, or consists essentially of a heavy chain having at least 96%, at least 97%, at least 98%, at least 99%, or 100% identity to the amino acid sequence of SEQ ID NO: 67 and a light chain having at least 96%, at least 97%, at least 98%, at least 99%, or 100% identity to the amino acid sequence of SEQ ID NO: 72.

In some embodiments, an antibody of the present disclosure comprises, consists of, or consists essentially of a heavy chain having at least 96%, at least 97%, at least 98%, at least 99%, or 100% identity to the amino acid sequence of SEQ ID NO: 68 and a light chain having at least 96%, at least 97%, at least 98%, at least 99%, or 100% identity to the amino acid sequence of SEQ ID NO: 73.

In some embodiments, an antibody of the present disclosure comprises, consists of, or consists essentially of a heavy chain having at least 96%, at least 97%, at least 98%, at least 99%, or 100% identity to the amino acid sequence of SEQ ID NO: 69 and a light chain having at least 96%, at least 97%, at least 98%, at least 99%, or 100% identity to the amino acid sequence of SEQ ID NO: 74.

In some embodiments, an antibody of the present disclosure comprises, consists of, or consists essentially of a heavy chain having at least 96%, at least 97%, at least 98%, at least 99%, or 100% identity to the amino acid sequence of SEQ ID NO: 70 and a light chain having at least 96%, at least 97%, at least 98%, at least 99%, or 100% identity to the amino acid sequence of SEQ ID NO: 75.

In some embodiments, an antibody of the present disclosure comprises, consists of, or consists essentially of a heavy chain having at least 96%, at least 97%, at least 98%, at least 99%, or 100% identity to the amino acid sequence of SEQ ID NO: 71 and a light chain having at least 96%, at least 97%, at least 98%, at least 99%, or 100% identity to the amino acid sequence of SEQ ID NO: 75.

In some embodiments, an antibody of the present disclosure comprises, consists of, or consists essentially of a heavy chain having the amino acid sequence of SEQ ID NO: 67 and a light chain having the amino acid sequence of SEQ ID NO: 72.

In some embodiments, an antibody of the present disclosure comprises, consists of, or consists essentially of a heavy chain having the amino acid sequence of SEQ ID NO: 68 and a light chain having the amino acid sequence of SEQ ID NO: 73.

In some embodiments, an antibody of the present disclosure comprises, consists of, or consists essentially of a heavy chain having the amino acid sequence of SEQ ID NO: 69 and a light chain having the amino acid sequence of SEQ ID NO: 74.

In some embodiments, an antibody of the present disclosure comprises, consists of, or consists essentially of a heavy chain having the amino acid sequence of SEQ ID NO: 70 and a light chain having the amino acid sequence of SEQ ID NO: 75.

In some embodiments, an antibody of the present disclosure comprises, consists of, or consists essentially of a heavy chain having the amino acid sequence of SEQ ID NO: 71 and a light chain having the amino acid sequence of SEQ ID NO: 75.

In some embodiments, an antibody or antigen-binding fragment thereof disclosed herein comprises, consists essentially of, or consists of a heavy chain variable domain sequence and a light chain variable domain sequence having at least 96% at least 97%, at least 98%, or at least 99% identity to an amino acid sequence in Table 9, 10, or 13.

In some embodiments, the antibody or antibody fragment disclosed herein comprises, or consists essentially of, or consists of a heavy chain variable domain sequence and/or a light chain variable domain sequence having an amino acid sequence disclosed in Table 9, 10, or 13.

In some embodiments, the antibody or antigen-binding fragment thereof is a monoclonal antibody. In some embodiments, the antibody is an IgG antibody.

In some embodiments, the antigen-binding fragment thereof is a recombinant single chain fragment variable or single chain variable domain fragment (scFv) antibody, a Fab fragment, a F(ab′)2 fragment, or a variable domain fragment (Fv fragment). In some embodiments, the antigen-binding fragment thereof is a recombinant scFv antibody. In some embodiments, the antigen-binding fragment thereof is a recombinant Fv fragment. In some embodiments, the antigen-binding fragment thereof (e.g., Fv fragment) comprises the amino acid sequence of one or more of: SEQ ID NOs: 10-18 or an amino acid sequence disclosed in Table 9, 10, or 13. In some embodiments, the antigen-binding fragment thereof (e.g., scFv or Fv fragment) comprises an amino acid sequence substantially identical (e.g., at least 80%, 85%, 90%, 92%, 95%, 96%, 97%, 98%, 99% or higher) to any of the amino acid sequences disclosed in Table 9, 10, or 13.

In some embodiments, the Fv fragment further comprises a linker domain to generate a single chain variable fragment (scFv). In one embodiment, the linker domain is operably linked to the heavy chain variable domain and the light chain variable domain. In some embodiments, the light and heavy chain variable regions are contiguously linked via a short flexible polypeptide linker, and capable of being expressed as a single chain polypeptide, and the scFv retains the specificity of the intact antibody from which it is derived. In one embodiment, the flexible polypeptide linker includes, but are not limited to, (Gly4 Ser)4 or (Gly4 Ser)3. In another embodiment, the linker includes multiple repeats of (Gly2Ser), (GlySer) or (Gly3Ser).

### Constant Region of an Antibody

In one aspect, technologies of the present disclosure provide an antibody or an antigen-binding fragment thereof comprising a heavy chain immunoglobulin variable domain (VH) and a light chain immunoglobulin variable domain (VL) as disclosed herein, and a Fc domain of any isotype, e.g., but are not limited to mouse IgG (including mouse IgG1, IgG2A, IgG2B, IgG2C, and IgG3). The constant region of antibodies can also be varied.

Additional exemplary constant regions are disclosed in Table 9 and Table 10.

In some embodiments, an antibody or antigen-binding fragment thereof of the present disclosure comprises one or more heavy chain constant domains. In some embodiments, an antibody or antigen-binding fragment thereof of the present disclosure comprises one or more heavy chain constant domains having at least 96%, at least 97%, at least 98%, or at least 99% identity to an amino acid sequence selected from SEQ ID NO: 19 or SEQ ID NO: 20. In some embodiments, an antibody or antigen-binding fragment thereof of the present disclosure comprises one or more heavy chain constant domains having an amino acid sequence selected from SEQ ID NO: 19 or SEQ ID NO: 20.

In some embodiments, an antibody or antigen-binding fragment thereof of the present disclosure comprises one or more light chain constant domains. In some embodiments, an antibody or antigen-binding fragment thereof of the present disclosure comprises one or more light chain constant domains having at least 96%, at least 97%, at least 98%, or at least 99% identity to the amino acid sequence of SEQ ID NO: 21. In some embodiments, an antibody or antigen-binding fragment thereof of the present disclosure comprises one or more light chain constant domains having the amino acid sequence of SEQ ID NO: 21.

In some embodiments, an antibody or antigen-binding fragment thereof comprises a heavy chain immunoglobulin variable domain (VH) and a light chain immunoglobulin variable domain (VL) as disclosed here, and one or more constant regions selected from SEQ ID NOs: 19-21.

### Felinized Antibodies

In some embodiments, an antibody or antigen-binding fragment thereof of the present disclosure can be “felinized” or undergo “felinization.” In some such embodiments, a “felinized antibody” of the present disclosure comprises variable region residues responsible for antigen binding (i.e., residues of a complementarity determining region, abbreviated complementarity determining region, or any other residues that participate in antigen binding) derived from a non-feline species, while the remaining variable region residues (i.e., residues of the framework regions) and constant regions are derived, at least in part, from feline antibody sequences. A subset of framework region residues and constant region residues of a speciated antibody may be derived from non-feline sources. Variable regions of a speciated antibody are also described as speciated (i.e., a speciated light or heavy chain variable region). The non-speciated species is typically that used for immunization with antigen, such as mouse, rat, rabbit, non-human primate, or other non-feline mammalian species.

Framework residues may be derived from a naturally occurring feline antibody. Artificial framework sequences that represent a consensus among individual sequences may also be used. When selecting a framework region for felinization, sequences that are widely represented in felines may be preferred over less populous sequences. Additional mutations of the feline framework acceptor sequences may be made to restore murine residues believed to be involved in antigen contacts and/or residues involved in the structural integrity of the antigen-binding site, or to improve antibody expression.

Grafting of CDRs is performed by replacing one or more CDRs of an acceptor antibody (ex., a felinized antibody or other antibody comprising desired framework residues) with CDRs of a donor antibody (ex, a non-feline antibody). Acceptor antibodies may be selected based on similarity of framework residues between a candidate acceptor antibody and a donor antibody. For example, feline framework regions identified as having substantial sequence homology to each framework region of the relevant non-feline antibody, and CDRs of the non-feline antibody can be grafted onto the composite of the different feline framework regions.

Acceptor frameworks for grafting of CDRs or abbreviated CDRs may be further modified to introduce desired residues. For example, acceptor frameworks may comprise a heavy chain variable region of a feline consensus sequence, optionally with non-feline donor residues at one or more positions. Following grafting, additional changes may be made in the donor and/or acceptor sequences to optimize antibody binding and functionality.

### Derivatives of Antibodies and Antigen-Binding Fragments Thereof

One aspect of the present disclosure provides a “derivative” of an antibody or antigen-binding fragment thereof of the present disclosure. The term “derivative” refers to an antibody or antigen-binding fragment thereof that specifically binds to an antigen (Fel d 1) but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered first constant domain of the heavy chain (CH1), hinge, second constant domain of the heavy chain (CH2), third constant domain of the heavy chain (CH3) or fourth constant domain of the heavy chain (CH4) regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc.

In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art. See e.g., Shields et al., J. Biol. Chem. 277(30): 26733-26740 (2002); Davies J. et al., Biotechnology & Bioengineering 74(4): 288-294 (2001). Methods of altering carbohydrate contents are also known to those skilled in the art. See, e.g., Wallick et al. J. Exp. Med. 168(3): 1099-1109 (1988); Tao et al. J. Immunol. 143(8): 2595-2601 (1989); Routledge et al., Transplantation 60(8):847-53 (1995); Elliott et al., Nature Biotechnol. 21:414-21 (2003); Shield et al. J. Biol. Chem. 277(30): 26733-26740 (2002).

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### Labeled Antibodies and Antigen-Binding Fragments Thereof

In some embodiments, antibodies and antigen-binding fragments thereof of the present disclosure can be conjugated to or further comprise a label (e.g., a detectable label). In some embodiments, an antibody or antigen-binding fragment thereof of the present disclosure can be covalently or non-covalently associated with a label (e.g., a detectable label). In some embodiments, an antibody or antigen-binding fragment thereof of the present disclosure can be directedly or indirectly associated with a label (e.g., a detectable label). In some embodiments, an antibody or antigen-binding fragment thereof of the present disclosure can be associated with a label (e.g., a detectable label) via a linker. In some embodiments, an antibody or antigen-binding fragment thereof of the present disclosure can be fused or conjugated with a label (e.g., a detectable label). In some embodiments, an antibody or antigen-binding fragment thereof of the present disclosure is associated with a label (e.g., a detectable label) via a polypeptide terminus of the antibody or antigen-binding fragment thereof. In some embodiments, an antibody or antigen-binding fragment thereof of the present disclosure is associated with a label (e.g., a detectable label) via a nonterminal residue of the antibody or antigen-binding fragment thereof. In some embodiments, an antibody or antigen-binding fragment thereof of the present disclosure is associated with a label (e.g., a detectable label) via incorporation of the label into the molecular structure of the antibody or antigen-binding fragment thereof. In some embodiments, an antibody or antigen-binding fragment thereof of the present disclosure can be associated with a plurality of same or different labels (e.g., detectable labels) via any mechanism disclosed herein or otherwise known in the art, which same or different labels can be associated with the antibody or antigen-binding fragment thereof via same or different mechansims of association as disclosed herein or otherwise known in the art.

Detectable labels of the present disclosure, include, for example, a label that produces, is capable of producing, or is capable of contributing to production of a signal detectable by any means known in the art. In particular instances, a detectable label produces, is capable of producing, or is capable of contributing to production of a signal detectable by, without limitation, visual means, spectroscopic means, photochemical means, biochemical means, immunochemical means, electromagnetic means, radiochemical means, chemical means, fluorescence, chemifluoresence, electrochemilumenscence, or chemiluminescence. Detectable labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In some embodiments, a detectable label is a fluorescent moiety, radioactive moiety, paramagnetic moiety, chemiluminescent moiety, bioluminescent moiety, colorimetic label, polypeptide, enzyme, and/or ligand. Examples of detectable labels include, but are not limited to, green fluorescent protein (GFP), red fluorescent protein (RFP), rhodamine, rhodamine-derived detectable moieties, fluorescein, fluorescein-derived detectable moieties, naphthalene, naphthalene-derived detectable moieties, coumarin, coumarin-derived detectable moieties, phycobiliproteins and derivatives such as phycoerythrin and phycocyanin, luciferase, beta-galactosidase, chromophores, phenolphthalein, malachite green, nitroaromatics such as nitrophenyl, diazo dyes, dabsyl (4-dimethylaminoazobenzene-4′-sulfonyl), His tag, and biotin-binding moieties such as streptavidin or avidin. A detectable moiety can be a radioisotope or radiolabel (e.g. ³H, ¹⁴C, ³²P, ³⁵S, ¹²⁵I, zirconium-89 (89Zr), iodine-124 (124I), iodine-131 (131I), iodine-125 (125I), bismuth-212 (212Bi), bismuth-213 (213Bi), astatine-221 (221At), copper-67 (67Cu), copper-64 (64Cu), rhenium-186 (186Re), rhenium-188 (188Re), phosphorus-32 (32P), samarium-153 (153Sm), lutetium-177 (177Lu), technetium-99m (99mTc), gallium-67 (67Ga), indium-111 (111In), or thallium-201 (201Tl), or a radiolabeled molecule. A detectable label may be a small molecule, a fluorescent dye, or a compound that may be detected by x-rays or electromagnetic radiation. A detectable moiety can be a catalytic substrate of an enzyme, wherein activity of enzyme with substrate produces a detectable signal. Enzyme detectable moieties of the present invention include, without limitation, peroxidase (e.g., horseradish peroxidase), alkaline phosphatase (AP), glucose oxidase, or β-galactosidase. A detectable label may be a colloidal gold.

In some embodiments, an antibody or antigen-binding fragment thereof of the present disclosure further comprises or is conjugated to a colloidal gold. In some embodiments, an antibody or antigen-binding fragment thereof of the present disclosure further comprises or is conjugated to a dye. In some embodiments, an antibody or antigen-binding fragment thereof of the present disclosure further comprises or is conjugated to a horseradish peroxidase.

Those of skill in the art will appreciate that an antibody or antigen-binding fragment thereof of the present disclosure can be detectable, for example, by use of a second antibody or antigen-binding fragment (e.g., conjugated to or further comprising a label, e.g., a detectable label), for example, in methods such as immunohistochemistry and immunofluorescence.

### Nucleic Acids

In another aspect, the present disclosure provides a nucleic acid molecule (e.g., an isolated nucleic acid molecule) comprising, or consisting essentially of, or consisting of a nucleotide sequence encoding the CDR, heavy chain, light chain, scFV, antibody or antibody fragment as disclosed herein that are optionally detectably labeled. In some embodiments, the nucleic acid comprises a nucleotide sequence encoding an Fv fragment comprising an amino acid sequence selected from one or more of SEQ ID NOs: 10-18; or an amino acid sequence disclosed in Table 9, Table 10, or Table 13.

In some embodiments, the nucleic acid comprises a nucleotide sequence encoding a heavy chain variable domain sequence comprising an amino acid sequence selected from SEQ ID NOs: 10-14. In some embodiments, the nucleic acid comprises a nucleotide sequence encoding a heavy chain variable domain sequence comprising an amino acid sequence disclosed in Table 9 or 13. In some embodiments, the nucleic acid comprises a nucleotide sequence encoding a heavy chain variable domain sequence comprising an amino acid sequence having at least 70%, 80%, 90%, 95%, 96%, 97%, 98%, or 99% identity to the amino acid sequence disclosed in Table 9 or 13.

In some embodiments, the nucleic acid comprises a nucleotide sequence encoding a light chain variable domain sequence comprising an amino acid sequence selected from SEQ ID NOs: 15-18. In some embodiments, the nucleic acid comprises a nucleotide sequence encoding a light chain variable domain sequence comprising an amino acid sequence disclosed in Table 10 or Table 13. In some embodiments, the nucleic acid comprises a nucleotide sequence encoding a light chain variable domain sequence comprising an amino acid sequence having at least 70%, 80%, 90%, 95%, 96%, 97%, 98%, or 99% identity to the amino acid sequence disclosed in Table 10 or Table 13.

In one aspect, the present disclosure provides an isolated polynucleotide comprising a nucleic acid sequence encoding the heavy chain variable region of the antibody or antigen-binding fragment thereof disclosed herein and/or a nucleic acid sequence encoding the light chain variable region of the antibody or antigen-binding fragment thereof disclosed herein. In some embodiments, the isolated polynucleotide comprises a nucleic acid sequence encoding the heavy chain variable region of the antibody or antibody fragment disclosed in Table 11 and/or a nucleic acid sequence encoding the light chain variable region of the antibody or antibody fragment disclosed in Table 12. In some embodiments, the isolated polynucleotide comprises a nucleic acid sequence having at least 70%, 80%, 90%, 95%, 96%, 97%, 98%, or 99% identity to a nucleic acid sequence encoding the amino acid sequence of a heavy chain variable region of the antibody or antibody fragment disclosed in Table 11 and/or a nucleic acid sequence having at least 70%, 80%, 90%, 95%, 96%, 97%, 98%, or 99% identity to a nucleic acid sequence encoding the amino acid sequence of a light chain variable region of an antibody or antibody fragment disclosed in Table 12.

In some embodiments, the nucleic acid comprises a nucleic acid sequence encoding a heavy chain variable domain comprising one, two, or three CDRs comprising an amino acid sequence as disclosed in Table 13. In some embodiments, the nucleic acid comprises a nucleic sequence encoding a light chain variable domain comprising one, two, or three CDRs comprising an amino acid sequence as disclosed in Table 13.

In some embodiments, the isolated polynucleotide comprises a nucleic acid sequence having at least 70%, 80%, 90%, 95%, 96%, 97%, 98%, or 99% identity to a nucleic acid sequence of any one or more of SEQ ID NOs: 31-41 or 66. In some embodiments, the isolated polynucleotide comprises a nucleic acid sequence of any one or more of SEQ ID NOs: 31-41 or 66.

The polynucleotides can be DNA or RNA, and can be operably linked to expression elements for transcription, translation or replication of the polynucleotides. Such include, for example promoters and enhancer elements, as are known in the art. The polynucleotides can be used for recombinant production of the polynucleotides or antibodies and fragments thereof as disclosed herein.

### Vectors

In another aspect, the present disclosure provides a vector comprising the nucleic acid molecule comprising, or consisting essentially of, or consisting of a nucleotide sequence encoding an antibody or antigen-binding fragment thereof disclosed herein. Many expression vectors are available and known to those of skill in the art and can be used for expression of antibodies and antigen-binding fragments thereof described herein. The choice of expression vector will be influenced by, for example, the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag or a myc tag, hemagglutinin tag or a tag for purification, for example, a GST fusion, and a sequence for directing protein secretion and/or membrane association.

Expression of the antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are described herein.

Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 115-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of the antibody, or antigen-binding fragment thereof, in host cells. A typical expression cassette contains a promoter operably linked to the nucleic acid sequence encoding the antibody chain (or antigen-binding fragment thereof) and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the antibodies and antigen-binding fragments thereof provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of host cells can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the antibody or antigen-binding fragment thereof can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

Non-viral approaches can also be employed for the expression of a protein (e.g., antibody, antigen-binding fragment thereof) in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transient expression may be obtained by RNA electroporation.

cDNA expression can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used, regulation can be mediated by the cognate regulatory sequences or, if desired, by regulatory sequences derived from a heterologous source, including any of the promoters or regulatory elements described above.

### Methods of Production an Antibody or Antigen-Binding Fragment Thereof

### Recombinant Host Cell

One aspect of the present disclosure provides a recombinant host cell comprising a nucleic acid molecule comprising, or consisting essentially of, or consisting of a nucleotide sequence encoding the antibody or antigen-binding fragment thereof disclosed herein or a vector comprising the nucleic acid molecule comprising a nucleotide sequence encoding the antibody or antigen-binding fragment thereof disclosed herein. The nucleic acid can be, e.g., present in the genome of the cell and/or present in a vector for expression of an antibody or antigen-binding fragment thereof. In some embodiments, the recombinant host cell produces the antibody or antigen-binding fragment thereof disclosed herein. In some embodiments, the antibody or antigen-binding fragment thereof disclosed herein binds to (e.g., specifically binds to) Fel d 1. In some embodiments, a host cell or recombinant host cell comprises the vector disclosed herein. In some embodiments, the host cell is a prokaryotic cell. In some embodiments, the host cell is a eukaryotic cell, such as for example an HEK 293 cell.

### Generating an Antibody or Antigen-Binding Fragments Thereof

In one aspect, the present disclosure provides a method of producing an antibody or an antigen-binding fragment thereof of the present disclosure. Methods of producing an antibody or antigen-binding fragment thereof of the present disclosure can include producing a recombinant host cell comprising a nucleic acid comprising a nucleotide sequence encoding the antibody or antigen-binding fragment thereof disclosed herein. A method of producing an antibody or an antigen-binding fragment thereof of the present disclosure can further comprise culturing a cell including a nucleic acid comprising a nucleotide sequence encoding an antibody or antigen-binding fragment thereof as disclosed herein under conditions that permit cell growth and/or cell division, production of an antibody or antigen-binding fragment thereof as disclosed herein, or both. A method of the present invention can further include isolating an antibody or antigen-binding fragment thereof as disclosed herein from a cell culture. Methods of producing nucleic acids, producing cells, culturing cells, and isolating antibodies from culture are known in the art.

For example, in some embodiments, monoclonal antibodies of the present disclosure may be isolated from phage antibody libraries using the techniques described in Clackson et al., Nature, 352:624-628 (1991) and Marks et al., J. Mol. Biol., 222:581-597 (1991).

The methods for generating monoclonal antibodies generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art.

The amount of antigen used in the production of antibodies can vary upon the nature of the immunogen as well as the animal used for immunization. A variety of routes can be used to administer the antigen (subcutaneous, intramuscular, intradermal, intravenous and intraperitoneal). The production of antibodies may be monitored by sampling blood of the immunized animal at various points following immunization. An additional (e.g., second injection, third injection), referred to as a booster or booster injection, also may be given. The process of boosting and titering is repeated until a suitable titer is achieved. When a desired level of immunogenicity is obtained, the immunized animal can be bled and the serum isolated and stored, and/or the animal can be used to generate antibodies.

For example, the monoclonal antibodies may also be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins.

### Compositions

Another aspect of the present disclosure provides a composition comprising, or consisting essentially of, or consisting of one or more antibodies or antigen-binding fragments thereof disclosed herein; the nucleic acid, or the vector described herein. In some embodiments, the composition is a pharmaceutically acceptable composition, and optionally can comprise other therapeutic agents for combination therapy.

The present disclosure provides pharmaceutical compositions comprising, or consisting essentially of, or consisting of felinized antibodies or antigen-binding fragments thereof of the present disclosure. In some embodiments, such pharmaceutical compositions comprise a therapeutically effective amount of a felinized antibody or an antigen-binding fragment thereof of the present disclosure and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the subject. The formulation should suit the mode of administration, which can be oral, intravenous, buccal, systemic, nasal, intraarterial, intrabuccal, intranasal, ocular, nebulized, injection, infusion, bronchial inhalation, inhalation, insufflation, intra-rectal, transdermal, rectal, vaginal, topical or delivered by mechanical ventilation.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can further comprise an additional agent for the augmentation of the antibody response or treatment.

### Methods of Administration and Treatment

In one aspect, the present disclosure contemplates felinized antibodies or antigen-binding fragments thereof for reducing (e.g., compared to an appropriate reference standard) the level of active Fel d 1 in or on a subject (e.g., a cat) comprising administering to the subject a pharmaceutical composition of the present disclosure. In some embodiments, the level of active Fel d 1 in or on a subject is reduced relative to the level of active Fel d 1 in or on the subject prior to treatment with a pharmaceutical composition of the present disclosure. In some embodiments, the subject is a cat (e.g., a domestic cat). The effectiveness of such methods can be evaluated using detection and/or quantification methods described herein or any assay known in the art for evaluating levels of Fel d 1.

Technologies of the present disclosure (e.g., pharmaceutical compositions) can be administered to a subject (e.g., a cat) by any suitable means which include, but are not limited to, parenteral route of administration.

In some embodiments, composition(s) as described herein are administered to the subject (e.g., a cat) parenterally. Non-limiting examples of parenteral administration include intravenous, intramuscular, intraarterial, subcutaneous, and intraperitoneal administration. In one embodiment, a composition(s) described herein is administered intravenously. In one embodiment, a composition(s) described herein is administered intraperitoneally. In one embodiment, a composition(s) described herein is administered intramuscularly. In one embodiment, a composition(s) described herein is administered subcutaneously.

An appropriate dosage of a pharmaceutical composition as described herein will vary with the particular level of Fel d 1 in a given subject (e.g., cat), the age, weight, and physical condition of the subject in need of treatment, the route of administration, the duration of the treatment, the responsiveness of the subject being treated, the nature of the concurrent or combination therapy (if any), the specific route of administration and like factors within the knowledge and expertise of a health (e.g., veterinary) practitioner. In certain embodiments, a maximal tolerable dose of a pharmaceutical composition as described herein is to be used, that is, the highest safe dose according to sound medical judgement. In preferred embodiments, a pharmaceutical composition as described herein is to be administered in effective amounts. An effective amount is, for example, a dose of the composition(s) sufficient to provide a medically desirable result. A medically desirable result, for example, for a subject (e.g., cat) may be an amount that reduces the amount of active Fel d 1 in and/or on the subject (e.g., compared to an appropriate reference standard).

Various dosing schedules of the pharmaceutical compositions described herein are contemplated including single administration or multiple administrations over a period of time. The therapeutic regimen for use in the methods described herein may include administration of a composition as described herein once a day, once every two days, once every three days, once every four days, once every five days, once every six days, twice a week, once every week, once every two weeks, once every three weeks, once every month or 4 weeks, once every six weeks, once every two months or eight weeks, or once every three months or twelve weeks. In certain embodiments, the subject receives a single dose of any therapy described herein. In certain embodiments, the subject receives from at least two, at least three, at least four, at least five, at least six, at least eight, or at least ten doses of any therapy described herein. In certain embodiments, a therapy described herein is administered daily, every other day, or two times a week. In certain embodiments, a therapy described herein is administered for a period of time, such as one week, two weeks, three weeks, four weeks, six weeks, two months, three months, four months, five months, six months, or one year.

Methods of Detection and/or Quantification

The present disclosure provides, among other things, methods of detecting and/or quantifying Fel d 1 in a sample, the method comprising, consisting essentially of, or consisting of contacting the sample with an antibody or antigen-binding fragment thereof disclosed herein.

In some embodiments, methods of detecting and/or quantifying Fel d 1 in a sample comprises, consists essentially of, or consists of contacting the sample with an antibody or antigen-binding fragment thereof of the present disclosure; and detecting Fel d 1 in the sample by detecting binding of the antibody or antigen-binding fragment thereof to the Fel d 1 in the sample.

In some embodiments, the sample is a biological sample. In some embodiments, the sample is a biological sample isolated from a feline (e.g., a domestic cat). In some such embodiments, the biological sample is isolated from one or more of saliva, anal glands, urine, sebaceous glands, skin, blood, serum, and fur of a feline (e.g., a domestic cat). In some embodiments, the feline is of the species Felis catus.

In some embodiments, the sample is an environmental sample. Environmental samples can include, for example, industrial samples, such as those isolated from surfaces (e.g., countertops, tables, furniture) and the environment.

In some embodiments, detecting Fel d 1 in the sample by detecting binding of the antibody or antigen-binding fragment thereof to the Fel d 1 in the sample comprises use of enzyme-linked immunosorbent assay (ELISA), lateral flow assay, immunohistochemistry, immunofluorescence, or Western blot. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing Fel d 1, and contacting the sample with a first antibody or antigen-binding fragment thereof in accordance with the present disclosure, as the case may be, under conditions effective to allow the formation of immunocomplexes.

These methods can include methods for purifying Fel d 1 or antigenic fragments thereof from a sample. The antibody or antigen-binding fragment thereof will preferably be linked to a solid support, such as in the form of a column matrix, and the sample suspected of containing the Fel d 1 or an antigenic fragment thereof will be applied to the immobilized antibody or antigen-binding fragment thereof. The unwanted components will be washed from the column, leaving the Fel d 1 or antigenic fragment thereof immunocomplexed to the immobilized antibody or antigen-binding fragment thereof, which can optionally be collected.

The antibody or antigen-binding fragment thereof employed in the detection and/or quantification may itself be linked to a label (e.g., a detectable label), wherein one would then simply detect this label, thereby allowing the amount of the first complexe (e.g., a complex comprising an antibody or antigen-binding fragment of the disclosure, a “first” antibody or antigen-binding fragment thereof”, and Fel d 1) in the composition to be determined. Alternatively, a first antibody or antigen-binding fragment thereof that becomes bound Fel d 1 may be detected by means of a second binding ligand that has binding affinity for the antibody (e.g., a secondary antibody). In these cases, the second binding ligand may be linked to a detectable label. The immune complex can be contacted with the labeled, secondary binding ligand, or antibody, under effective conditions and for a period of time sufficient to allow the formation of a second complex (e.g., comprising Fel d 1, the first antibody or antigen-binding fragment thereof, and the second binding ligand). The second complex is then generally washed to remove any non-specifically bound labeled secondary antibodies or ligands, and the remaining label in the secondary immune complexes is then detected.

Further methods include the detection of first by a two-step approach. A second binding ligand, such as an antibody that has binding affinity for the antibody, is used to form the second complex, as described above. After washing, the second complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired. In some embodiments, the third binding ligand is a secondary antibody.

In some embodiments, methods of detecting or quantifying Fel d 1 in a sample of the present disclosure comprise: (a) contacting the sample with a first antibody or antigen-binding fragment thereof selected from an anti-Fel d 1 antibody or antigen-binding fragment thereof of the present disclosure, thereby producing a first complex comprising the Fel d 1 and the first antibody or antigen-binding fragment thereof; (b) contacting the first complex with a second antibody or antigen-binding fragment thereof selected from an anti-Fel d1 antibody or antigen-binding fragment thereof of the present disclosure, thereby producing a second complex comprising the Fel d 1, the first antibody or antigen-binding fragment thereof, and the second antibody or antigen-binding fragment thereof; wherein either of the first antibody or antigen-binding fragment thereof or the second antibody or antigen-binding fragment thereof comprises a label capable of producing a signal; and (c) measuring the signal.

In some embodiments, the first antibody or antigen-binding fragment thereof and the second antibody or antigen-binding fragment thereof are from different species. In some embodiments, the first antibody or antigen-binding fragment thereof and the second antibody or antigen-binding fragment thereof are from the same species.

In some embodiments, the first antibody or antigen-binding fragment thereof and the second antibody or antigen-binding fragment thereof are different isotypes or subclasses. In some such embodiments, the different subclasses are selected from mouse IgG1, IgG2A, IgG2B, IgG2C, and IgG3.

In some embodiments, the first antibody or antigen-binding fragment thereof and the second antibody or antigen-binding fragment thereof comprise the same CDRs. In some embodiments, the first antibody or antigen-binding fragment thereof and the second antibody or antigen-binding fragment thereof comprise different CDRs.

In some embodiments, the first antibody or antigen-binding fragment thereof, the second antibody or antigen-binding fragment thereof, or both is an IgG, optionally an IgG1.

In some embodiments, the first antibody or antigen-binding fragment thereof and the second antibody or antigen-binding fragment thereof are from different species.

In some embodiments, the first antibody or antigen-binding fragment thereof comprises a label capable of producing a signal (e.g., a detectable label). In some embodiments, the second antibody or antigen-binding fragment thereof comprises a label capable of producing a signal (e.g., a detectable label). In some embodiments, the label (e.g, detectable label) comprises colloidal gold, horseradish peroxidase, or a dye. In some embodiments, the dye is a fluorescent label.

In some embodiments, the first antibody or antigen-binding fragment thereof comprises: (a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; (b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; (c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or (d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the second antibody or antigen-binding fragment thereof comprises: (a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; (b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; (c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or (d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the first antibody or antigen binding fragment thereof comprises a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; and the second antibody or antigen-binding fragment thereof comprises a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the first antibody or antigen binding fragment thereof comprises a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65; and the second antibody or antigen-binding fragment thereof comprises a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62.

In some embodiments, the first antibody or antigen binding fragment thereof comprises a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65; and the second antibody or antigen-binding fragment thereof comprises a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64.

In some embodiments, the first antibody or antigen binding fragment thereof comprises a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; and the second antibody or antigen-binding fragment thereof comprises a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

In some embodiments, the first antibody or antigen binding fragment thereof comprises a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63; and the second antibody or antigen-binding fragment thereof comprises a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62.

In some embodiments, the first antibody or antigen-binding fragment thereof comprises a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62; and the second antibody or antigen-binding fragment thereof comprises a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63.

In some embodiments, the first antibody or antigen-binding fragment thereof comprises the heavy chain variable domain sequence of SEQ ID NO: 13 and the light chain variable domain sequence of SEQ ID NO: 18; and the second antibody or antigen-binding fragment thereof comprises the heavy chain variable domain sequence of SEQ ID NO: 10 and the light chain variable domain sequence of SEQ ID NO: 15.

In some embodiments, the first antibody or antigen-binding fragment thereof comprises the heavy chain variable domain sequence of SEQ ID NO: 10 and the light chain variable domain sequence of SEQ ID NO: 15; and the second antibody or antigen-binding fragment thereof comprises the heavy chain variable domain sequence of SEQ ID NO: 13 and the light chain variable domain sequence of SEQ ID NO: 18.

In some embodiments, the first antibody or antigen-binding fragment thereof comprises the heavy chain variable domain sequence of SEQ ID NO: 12 and the light chain variable domain sequence of SEQ ID NO: 17; and the second antibody or antigen-binding fragment thereof comprises the heavy chain variable domain sequence of SEQ ID NO: 10 and the light chain variable domain sequence of SEQ ID NO: 15.

In some embodiments, the first antibody or antigen-binding fragment thereof comprises the heavy chain variable domain sequence of SEQ ID NO: 10 and the light chain variable domain sequence of SEQ ID NO: 15; and the second antibody or antigen-binding fragment thereof comprises the heavy chain variable domain sequence of SEQ ID NO: 12 and the light chain variable domain sequence of SEQ ID NO: 17.

In some embodiments, the first antibody or antigen-binding fragment thereof comprises the heavy chain variable domain sequence of SEQ ID NO: 11 and the light chain variable domain sequence of SEQ ID NO: 16; and the second antibody or antigen-binding fragment thereof comprises the heavy chain variable domain sequence of SEQ ID NO: 10 and the light chain variable domain sequence of SEQ ID NO: 15.

In some embodiments, the first antibody or antigen-binding fragment thereof comprises the heavy chain variable domain sequence of SEQ ID NO: 10 and the light chain variable domain sequence of SEQ ID NO: 15; and the second antibody or antigen-binding fragment thereof comprises the heavy chain variable domain sequence of SEQ ID NO: 11 and the light chain variable domain sequence of SEQ ID NO: 16.

In some embodiments, methods of detecting and/or quantifying Fel d 1 of the present disclosure may be advantageously formatted for non-healthcare (e.g., home) use, including, for example, as a lateral flow assay (see below). These assays may be packaged in the form of a kit with appropriate reagents and instructions or a link (e.g., QR code) to instructions.

### Lateral Flow Assays

Lateral flow assays, also known as lateral flow immunochromatographic assays, are simple devices intended to detect the presence (or absence) of a target analyte (e.g., Fel d 1) in a sample (matrix) without the need for specialized and costly equipment.

The technology is based on a series of capillary beds, such as pieces of porous paper or sintered polymer. Each of these elements has the capacity to transport fluid (e.g., a sample, e.g., a biological sample, such as urine from a cat) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles in a salt-sugar matrix that contains everything to support a chemical reaction between the target molecule (e.g., an antigen, e.g., Fel d 1) and its chemical partner (e.g., antibody or antigen-binding fragment thereof that specifically binds to Fel d 1) that has been immobilized on the particle's surface. While the sample fluid dissolves the salt-sugar matrix, it also dissolves the particles and in one combined transport action the sample and conjugate mix while flowing through the porous structure. In this way, the target analyte binds to the particles while migrating further through the third capillary bed. This material has one or more areas (often called stripes) where a third molecule has been immobilized by the manufacturer. By the time the sample-conjugate mix reaches these strips, target analyte has been bound on the particle and the third ‘capture’ molecule binds (e.g., antibody or antigen-binding fragment thereof that specifically binds to Fel d 1) the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which a target analyte molecule has been immobilized. After passing these reaction zones, the fluid enters the final porous material—the wick—that simply acts as a waste container. Lateral flow assays can operate as either competitive or sandwich assays. Lateral flow assays are disclosed in U.S. Pat. No. 6,485,982.

In some embodiments, the present disclosure provides a lateral flow device comprising one or more antibodies or antigen-binding fragments thereof of the present disclosure.

In some embodiments, a lateral flow assay utilizing one or more antibodies or antigen-binding fragments thereof of the present disclosure is a competitive lateral flow assay. In some embodiments, a lateral flow assay utilizing one or more antibodies or antigen-binding fragments thereof of the present disclosure is a sandwich lateral flow assay.

### Kits

The present disclosure provides, among other things, a kit including one or more of: (a) an antibody or antigen-binding fragment thereof as described herein; (b) one or more reagents for use in a technique capable of detecting binding of Fel d 1 by an antibody or antigen-binding fragment thereof of the present disclosure; (c) one or more reagents for use in administration of a felinized antibody or antigen-binding fragment thereof of the present disclosure (e.g., pharmaceutical composition of the present disclosure); (d) instructions (e.g., or a link to instructions, such as a QR code) for a technique capable of detecting binding of Fel d 1 to an antibody or antigen-binding fragment thereof of the present disclosure; and/or (e) instructions for administering a felinized antibody or antigen-binding fragment of the present disclosure (e.g., a pharmaceutical composition comprising such a felinized antibody) to a subject (e.g., a cat).

In some embodiments, a kit of the present disclosure comprises one or more antibodies or antigen-binding fragments thereof as described herein. In a preferred embodiment, at least one antibody or antigen-binding fragment thereof of the kit further comprises or is conjugated to a label (e.g., a detectable label). In some such embodiments, the label (e.g., detectable label is HRP or a fluorophore).

In some embodiments, a kit of the present disclosure comprises a lateral flow device comprising one or more antibodies or antigen-binding fragments thereof of the disclosure.

In some embodiments, a kit of the present disclosure comprises one or more isolated nucleotides as described herein. In some embodiments, a kit of the present disclosure comprises one or more compositions as described herein. In some embodiments, a kit of the present disclosure comprises a recombinant host cell as described herein.

In some embodiments, a kit of the present disclosure can comprise one or more reagents for detecting Fel d 1, e.g., one or more reagents for use in a technique capable of detecting binding of Fel d 1 by an antibody or antigen-binding fragment thereof described herein or known in the art. In some embodiments, a reagent for use in a technique capable of detecting binding of Fel d 1 by an antibody or antigen-binding fragment thereof described herein or known in the art comprises blocking buffer, a wash buffer, Fel d 1 polypeptide, and/or a positive control.

In some embodiments, a reagent for use in a technique capable of detecting binding of Fel d 1 by an antibody or antigen-binding fragment thereof as described herein is a secondary antibody.

In some embodiments, an antibody or antigen-binding fragment of the present disclosure is a mouse anti-Fel d 1 antibody or antigen-binding fragment thereof and the secondary antibody is an anti-mouse secondary antibody. In some embodiments, an antibody or antigen-binding fragment of the present disclosure is a felinized anti-Fel d 1 antibody or antigen-binding fragment thereof and the secondary antibody is an anti-feline secondary antibody.

In some embodiments, the kits of the present disclosure comprise a means for containing (container means) the individual components of the kit (e.g., antibody, antigen, and any other reagent) in close confinement for commercial sale. The container means of the kits will generally include at least one vial, test tube, flask, bottle, syringe or other container means, into which the individual kit component may be placed, or preferably, suitably aliquoted. Such containers may include injection or blow-molded plastic containers into which the desired vials are retained.

### EXAMPLES

These examples are provided for illustrative purposes only and not to limit the scope of the claims provided herein.

### Example 1: Materials and Methods

Fel d 1 plate trapped antigen immobilised directly to the ELISA: Fel d 1 antigen was coated on Greiner Bio-One Microlon assay plate at 1 μg/ml overnight at 4° C. Subsequently, 1% milk protein was utilized for blocking for 30 minutes at room temperature. Test 1 sera was titrated to 1:100 and diluted to 1:204,800 in a standard antigen ELISA and incubated for 2 hours in the assay at room temperature. Secondary antibodies, Sigma Aldrich A9044 1:20,000, Sigma Aldrich A2554 1:20,000 were diluted in in Phosphate Buffered Saline (PBS), 0.05% Tween, 0.1% dried skimmed milk, added to the assay plate, and incubated for 1 hour at room temperature. 3,3′,5,5′-Tetramethylbenzidine (TMB) substrate was added to the assay plate and incubated for 10 minutes at room temperature and an acid stop was completed. Washes were completed using PBS, 0.05% Tween 20.

Fel d 1 antigen Biotin capture: Antigen coated Protein G was coated on Greiner Bio-One Microlon assay plate at 1 μg/ml overnight at 4° C. Subsequently, 1% milk protein was utilized for blocking for 30 minutes at room temperature. Test 1 sera was titrated to 1:100 and triple diluted across the assay plate and incubated for 2 hours at room temperature. Fel d 1-biotin was diluted to 0.5 μg/ml in PBS, 0.05% Tween, 0.1% dried skimmed milk, added to the assay plate, and incubated for 2 hours at room temperature. Streptavidin-peroxidase (Sigma Aldrich S5512) was diluted to 0.25 μg/ml in PBS, 0.05% Tween, 0.1% dried skimmed milk, added to the assay plate, and incubated for 1 hour room temperature. TMB substrate was added to the assay plate and incubated for 10 minutes at room temperature and an acid stop was completed. Washes were completed using PBS, 0.05% Tween 20.

Titration of the purified Fel d 1 Monoclonal cell line antibodies was by Fel d 1 plate trapped antigen ELISA: Fel d 1 antigen (natural Fel d 1 antigen purified from cat hair, Indoor Biotechnologies, NA-FD1-1) was coated on Greiner Bio-One Microlon assay plate at 1 μg/ml overnight at 4° C. Subsequently, 1% milk protein in PBS was utilized for blocking for 30 minutes at room temperature. Purified antibodies (3000 ng/mL) were triple diluted down columns of the assay plate and incubated for 4 hours at room temperature. Secondary antibodies, Sigma Aldrich A9044 1:20,000 plus Sigma Aldrich A2554 1:20,000 were added to the assay plate and incubated for 1 hour at room temperature. TMB substrate was added to the assay plate and incubated for 10 minutes at room temperature and an acid stop was completed. Antibodies were diluted in PBS, 0.05% Tween, 0.1% dried skimmed milk unless otherwise stated. Washes were completed using PBS, 0.05% Tween 20.

Antibody Purification and Conjugation: Monoclonal supernatants were purified by protein G affinity chromatography and dialysed into physiological saline. Antibodies were conjugated to horse radish peroxidase (HRP) by oxidising antibody polysaccharide residues to aldehyde groups and coupling to HRP amine groups by reductive amination. Conjugates were dialysed into PBS and glycerol added to each conjugate. Conjugates were titrated in a protein G capture ELISA to confirm conjugation.

Chequerboard Assay to Identify Antibody Pairs: Each antibody was used as a capture antibody in combination with each of the antibody-HRP conjugates. Antibodies were coated at 2 μg/ml in carbonate/bicarbonate buffer, overnight at 4° C. Plates were blocked with PBS containing 1% milk protein, for 30 minutes at room temperature. Fel d 1 at 200 ng/ml in PBST containing 0.1% milk protein was added to wells and incubated for 1 hour at room temperature. Each of the MAb-HRP conjugates was diluted 1:500 in PBST containing 0.1% milk protein applied and incubated for 1 hour at room temperature. TMB substrate was applied and incubated for 15 minutes in the dark at room temperature. Acid stop was then applied.

Standard Curves from Paired Antibodies: Antibody pairs were investigated further by generating standard curves. The assay was similar to the chequerboard assay, except Fel d 1 standard curves were generated from 200 ng/ml doubling down to 0.098 ng/ml. Selected MAb-HRP conjugates were diluted 1:500 in PBST containing 0.1% milk protein.

Preparation of Gold Conjugates for ‘wet’ testing: Each tested monoclonal antibody was conjugated and diluted to 1 mg/mL in PBS. The required amount of antibody was added to 50 μL of the appropriate conjugation buffer in a 1.5 mL tube. 1 mL of 40 nm gold colloid (OD5) was then added to the tube and vortexed for 10 seconds. The tube was left to incubate at room temperature for 10 minutes. After 10 minutes 10 μl of 200 mg/ml BSA was added to the conjugate and mixed by inversion. The tube was left to incubate at room temperature for 30 minutes. After 30 minutes the conjugate was spun at 4000 g for 10 minutes and then the supernatant was removed. The gold was resuspended in 1 ml of gold drying buffer; conjugation buffer containing 5% sucrose (w/v), 3% BSA (w/v) and 1% Tween 20 (v/v).

Preparation of nitrocellulose (NC) membrane: Tested monoclonal antibodies were plotted onto NC. A test line of 1 mg/ml of antibody amended to contain 1% sucrose using a PBS solution containing 50% sucrose was plotted. A control line of 1 mg/ml of anti-mouse antibody amended to contain 1% sucrose using a PBS solution containing 50% sucrose was plotted. The Isoflow was used to plot the test and control lines at 7 mm and 13 mm respectively. The membranes Sartorius CN95, CN140 and MDI 200CNPH were investigated. Membranes were dried at 60° C., 10 mm/sec before sealed in foil pouches containing desiccant.

Lamination and preparation of ‘wet’ testing strips: 60 mm backing card was trimmed down to 40 mm. Nitrocellulose was placed at the bottom of the backing card, ensuring the bottom was flush with the bottom of the backing card. The sink pad was placed flush with the top edge of the backing card to give a 7 mm overlap onto the nitrocellulose. A handheld roller was used to ensure good contact of all material on the backing card. Bands were then cut into 5 mm lateral flow strips using the Kinematic programmable shear. Strips were stored in sealed foil pouches containing desiccant.

Testing procedure for ‘wet’ testing: The antigen was diluted to 10 μg/ml in PBST and then a 10-fold series dilution was carried out, until 0.001 μg/ml was reached. For a negative standard just PBST was run. 40 μl of the standard was aliquoted into the well of a 96 well low binding plate. 2 μl of the gold conjugate was added to the well and mixed with the standard via aspiration. A truncated lateral flow strip was added to the well and left to run for 10 minutes. After 10 minutes the strip was read using an Optricon cube reader and RFID card set to read a test line at 7 mm and a control line at 13 mm.

Preparation of conjugated pad. Following wet testing MAb 2 and MAb 20 gold conjugates were dried down on conjugate pad. Gold conjugates were prepared as above. Gold conjugates were sprayed onto 8951 (17 mm) conjugate pad using the Isoflow reagent dispenser. Gold conjugates were sprayed at 0.8 μl/mm, 0.4 μl/mm and 0.2 μl/mm spray rates with a nozzle height of 15 mm. Conjugate pads were dried in the Hedinair drying tunnel at 60° C. with a belt speed of 5 mm/sec. The sprayed band were sealed into foil pouches with dessicant.

Lamination and assembly of devices: The nitrocellulose membrane (CN140, 25 mm) was placed 20 mm from the bottom of the 60 mm backing card. A band of approx. 33 cm in length of sink pad (22 mm) was stuck down with the top edge being flush with the top of the backing card, giving a 7 mm overlap with the NC. A band of conjugate pad was stuck 5 mm from the bottom of the backing card to give a 2 mm overlap with the NC. Sample pad was stuck down with the bottom edge flush with the bottom edge of the backing card, giving a 5 mm overlap with the conjugate pad. A handheld rubber roller was used to ensure good contact of all materials on the backing card. Bands were cut into 5 mm wide lateral flow strips using the Kinematic programmable shear. Strips were placed into standard housing, which were passed through the roller closing devices set to a height of 49 mm. Devices were stored in sealed foil pouches containing desiccant.

Testing lateral flow devices: The antigen was diluted to 10 μg/ml in PBST and then a 10-fold series dilution was carried out, until 0.001 μg/ml was reached. For a negative standard just PBST was run. 80 μl of the standard was added to the port of the device. After 10 minutes the strip was read using an Optricon cube reader and RFID card set to read a test line at 7 mm and a control line at 13 mm.

### Example 2: Development of Mouse Monoclonal Cell Lines to Fel d 1

The present example demonstrates, among other things, the development of six stable monoclonal cell lines which, by ELISA, produced monoclonal antibodies which bind the Fel d 1 homologous antigen.

To develop mouse monoclonal cell lines to Fel d 1, following infectious agent screening by polymerase chain reaction (PCR), nine BALB/c mice were immunized with intent of following a standard 75-day protocol (Table 1) with best responding mice identified and selected for fusions.

TABLE 1
Mouse 75-day immunization protocol

| Day | | |
|---|---|---|
| Number | Procedure | Detail |
| 0 | Immunisation | Freunds complete adjuvant/aqueous emulsion |
| | | containing 30 μg of antigen (Natural Fel d 1 |
| | | (NA-FD1-1, Indoor Biotechnologies), dose |
| | | volume 50-100 μl, IP |
| 14 | Boost 1 | Freunds incomplete adjuvant/aqueous |
| | | emulsion containing 30 μg of antigen |
| | | (Natural Fel d 1 (NA-FD1-1, Indoor |
| | | Biotechnologies), dose |
| | | volume 50-100 μl, delivered IP |
| 28 | Boost 2 | Freunds incomplete/aqueous emulsion |
| | | containing 30 μg of antigen (Natural Fel d 1 |
| | | (NA-FD1-1, Indoor Biotechnologies), dose |
| | | volume 50-100 μl, delivered IP |
| 42 | Boost 3 | Freunds incomplete/aqueous emulsion |
| | | containing 30 μg of antigen (Natural Fel d 1 |
| | | (NA-FD1-1, Indoor Biotechnologies), dose |
| | | volume 50-100 μl, delivered IP |
| 49 | Test Bleed | Small test bleed to yield around 20-30 μl of |
| | | serum |
| 71* | Challenge | Boost 4, 30 ug Fel d 1 in PBS by IV* to |
| | | selected mice |
| 75 | Terminal Bleed & | Small terminal bleed to yield 50-80 μl of |
| | Splenectomy | serum. Spleen collected aseptically into tube |
| | | containing DMEM |

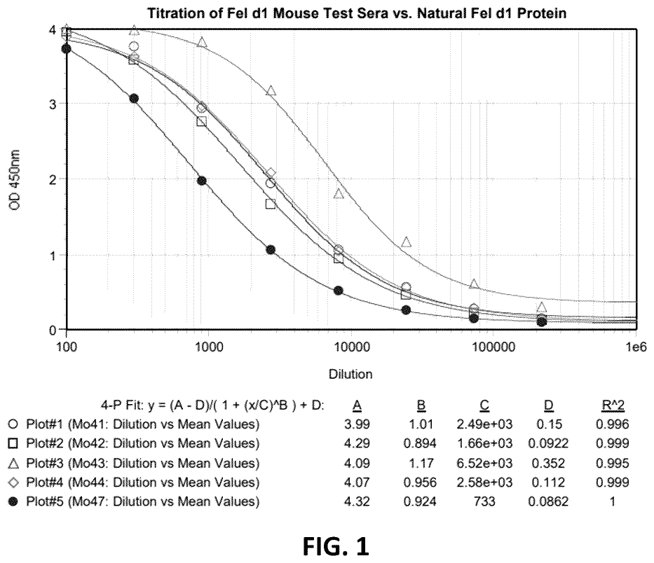

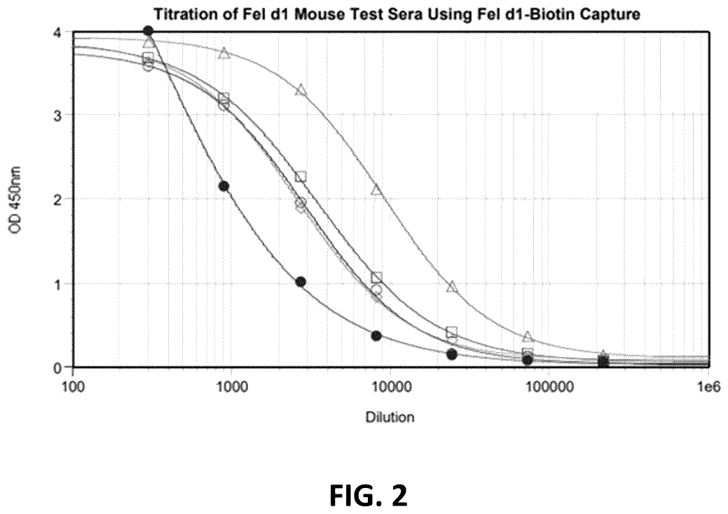

Four doses of natural Fel d 1 were to be administered by intraperitoneal injection (IP) at days 0, 14, 28 and 42 with an intravenous challenge (IV) on day 71 with spleen collection on day 75. However, following the second Fel d 1 boost (Day 28), four of the mice had a severe reaction to the immunogen and were culled. From the remaining five mice, the tail bleeds were collected (Test sera 1) and their immune response to native Fel d 1 was assessed by enzyme-linked immunosorbent assay (ELISA). Two formats of ELISA were chosen for this process: one wherein Fel d 1 plate trapped antigen was immobilized directly to the ELISA one and one wherein Fel d 1 antigen biotin capture was utilized. It was determined that the half-life titres of each mouse were similar for either ELISA format (FIG. 1, FIG. 2, Table 2).

TABLE 2
Comparison of test bleed one immune response to Fel d 1 native
immunogen when titrated against a) Fel d 1 plated trapped
antigen ELISA and b) Fel d 1 Biotin capture ELISA.

| | Plate trapped Fel d 1 | Fel d 1 Biotin |
|---|---|---|
| Mouse ID | Antigen ELISA | capture ELISA |
| 41 | 1:2,600 | 1:2,900 |
| 42 | 1:1,700 | 1:3,400 |
| 43 | 1:7,400 | 1:9,100 |
| 44 | 1:1,2600 | 1:2,600 |
| 45 | 1:1,1000 | 1:1,1000 |

For the third boost, the immunogen concentration of Fel d 1 was reduced to 15 ug for each mouse. Following immunization, three mice showed no discomfort whilst two showed a mild reaction but not at a severity level to require culling. Assessment of tail bleeds for immune response to Fel d 1 native antigen was as described previously. To mitigate risk of the mice dying at the final immunization (Fel d 1 challenge by IV) the mice were individually immunized over a staggered time frame with a reduced concentration of 15 ug Fel d 1 to the originally proposed 30 μg and by intraperitoneally injection (IP). Unfortunately, the first mouse (Mouse 42) required euthanizing within a couple of hours from boost. However, the spleen was collected and the fusion process described below was carried out. For the remaining mice, the process was continued with 15 ug Fel d1 mixed with adjuvant but by sub-cutaneous (SC) rather than IP. The inclusion this time of adjuvant was to obtain a slower release of the Fel d 1 and minimize potential for side-effects. Thereafter no adverse reactions were observed and the spleens of the four remaining mice were collected.

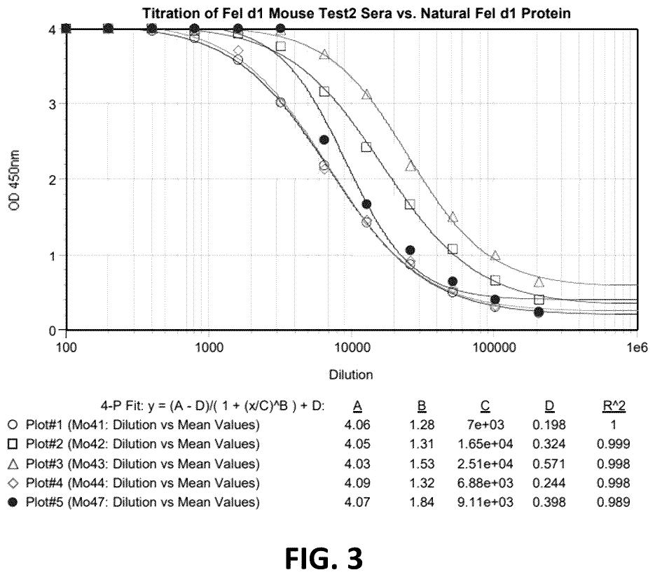

After the third boost, the tail bleeds were collected (test bleed 2) and the sera dilutions were screed by plate trapped Fel d 1 antibody ELISA. Results are shown in FIG. 3 and Table 3 and demonstrate that test bleed 2 titres were improved in comparison with test 1 sera titres.

TABLE 3
Comparison of Fel d 1 test 1 and test 2 sera (test
bleed 2) by plate trapped Fel d 1 antigen ELISA.

| Mouse ID | Test 1 Titre | Test 2 Titre |
|---|---|---|
| 41 | 1:2,600 | 1:7,000 |
| 42 | 1:1,700 | 1:16,500 |
| 43 | 1:7,400 | 1:25,100 |
| 44 | 1:2,600 | 1:6,900 |
| 47 | 1:1,000 | 1:9,100 |

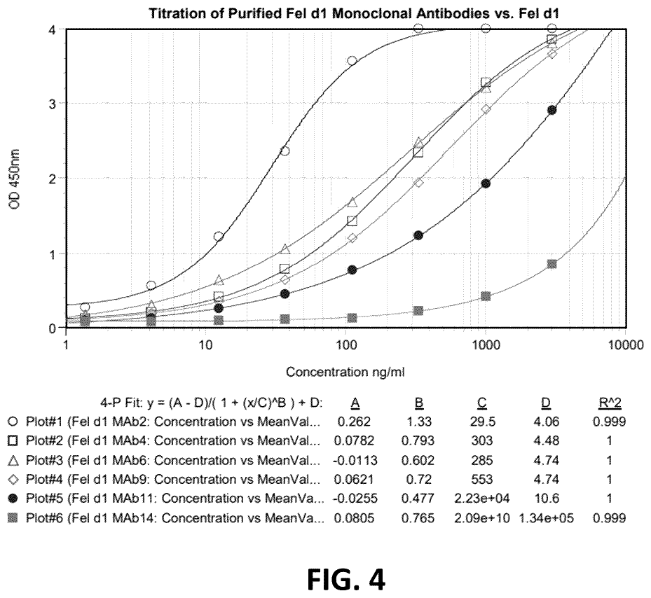

From all remaining live mice, B-lymphocytes were fused with NS0 myeloma cells using PEG-1500 and dispersed into 96-well plates. Hybrid cells were cultured using medium containing Hypoxanthine-Aminopterin-Thymidine (HAT) for selection. Once cell colonies were observed, the medium was changed as required. Fusion supernatants were screened by ELISA. Selected hybridoma cells were taken through rounds of cloning (by limited dilution) and assayed to establish stable monoclonal cell lines. Monoclonal cell lines were expanded, cells frozen, and supernatants purified by protein G affinity chromatography. Fusions resulted in six stable clones, summarized in Table 4. Titration of these by Fel d 1 plate trapped antigen ELISA (FIG. 4) resulted in half titre of between 553 ng ml⁻¹ to >3,0000 ng ml⁻¹ (Table 5).

TABLE 4
Mouse ID and resultant stable clones to Fel d 1 antigen

| | Final | Fusion | Stable clones | |
|---|---|---|---|---|
| Mouse ID | Boost | Efficiency | to Fel d 1 | MAb ID's |
| 41 | SC | 48 | — | |
| 42 | IP | 44 | 2 | Fel d 1 MAb2, 4 |
| 43 | SC | 46 | 2 | Fel d 1 MAb6, 9 |
| 44 | SC | 48 | — | |
| 47 | SC | 84 | 2 | Fel d 1 MAb11, 14 |

TABLE 5
Half-titres of monoclonal antibodies.

| Mouse ID | MAb 2 | MAb 4 | MAb 6 | MAb9 | MAb11 | MAb14 |
|---|---|---|---|---|---|---|
| Fel d 1 | 30 | 303 | 285 | 553 | >3,000 | >3,000 |
| half-titre | ng/ml | ng/ml | ng/ml | ng/ml | ng/ml | ng/ml |

Cryo-preserved cell lines (1 vial of each cell line) and purified monoclonal antibodies (1 mg scale) were produced.

### Example 3: Chequerboard ELISA to Identify Antibody Pairs

The present example demonstrates, among other things, identification of antibody pairs for use in, for example, an ELISA assay.

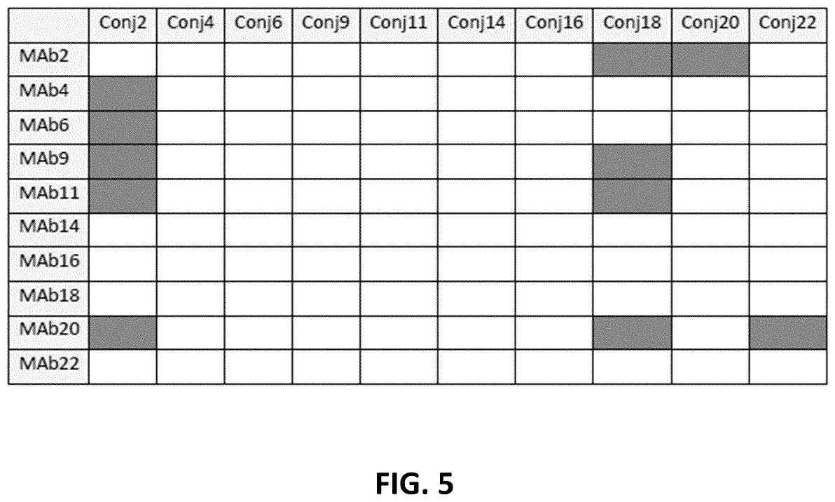

As described above, each antibody was used as a capture antibody in combination with each of the antibody-HRP conjugates. A visual representation of the chequerboard ELISA results are shown in FIG. 5. Such results demonstrated that: (i) MAb 2 forms antibody pairs with MAb-HRP conjugates 18 and 20; (ii) MAbs 4 and 6 form antibody pairs with MAb-HRP conjugate 2; (iii) MAbs 9 and 11 form antibody pairs with MAb-HRP conjugates 2 and 18; (iv) MAb 20 forms antibody pairs with MAb-HRP conjugates 2, 18 and 22; and (v) MAbs 14 and 16 do not appear to form antibody pairs.

### Example 4: Sandwich ELISA Standard Curves

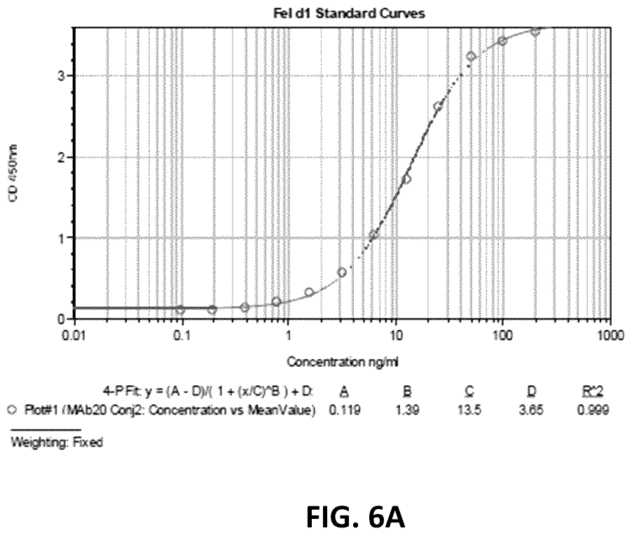

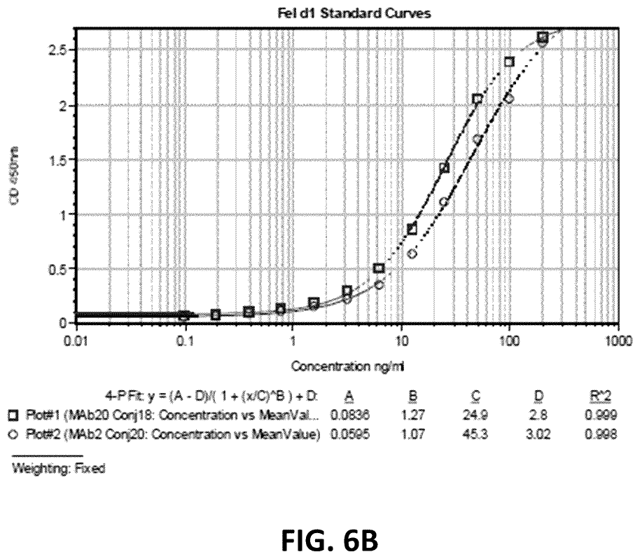

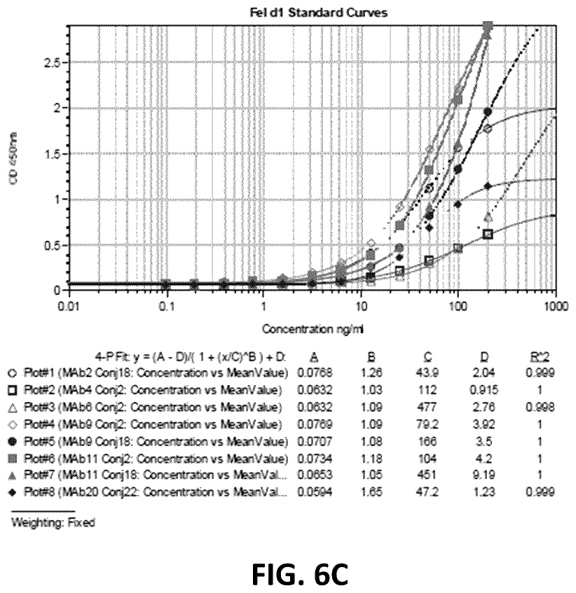

Standard curves were generated from all 11 antibody pairs identified in the chequerboard ELISA (Example 3). Results are shown in FIG. 6A-6C. Antibody pairs Fel d 1 MAb20 (capture) in combination with Fel d 1 MAb2 (detect) appeared to be the most sensitive combination, followed by Fel d 1 MAb20 (capture) in combination with Fel d 1 MAb18 (detect) and then Fel d 1 MAb2 (capture) in combination with Fel d 1 MAb20 (detect). Such results also identified several Fel d 1 antibody pairs suitable for use in sandwich assay formats. Based on these results, the following pairing options were assessed in lateral flow: (i) mAb 2 and 20; (ii) mAb 20 and 18; and (ii) mAb2 and 9. Each paring option was assessed in both orientations with respect to capture and detection.

### Example 5: ‘Wet’ Testing of Gold Conjugates

Gold conjugates for all tested monoclonal antibodies were prepared, pelleted by centrifugation, and resuspended in drying buffer (mAb 2: 20 mM [tris(hydroxymethyl)methylamino]propanesulfonic acid (TAPS) pH 8.5, 3% bovine serum albumin (BSA), 5% Sucrose & 1% Tween 20; mAb 9, 18, 20: 20 mM 2-[Bis(2-hydroxyethyl)amino]ethanesulfonic acid (BES) pH 7.8, 3% BSA, 5% Sucrose & 1% Tween 20). Each antibody was plotted at 1 mg/ml onto CN140 nitrocellulose membrane and placed onto backing card with sink pad to make truncated strips. Six different antibody pairings were tested.

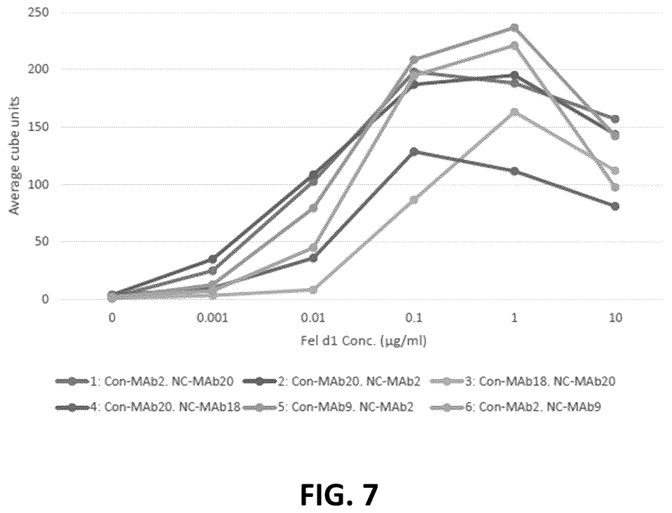

Stock Fel d 1 antigen was first diluted to 10 μg/ml in PBST (0.1% Tween 20) and then subsequently from this a 10-fold series dilution was carried out until 0.001 μg/ml was reached. Strips were run with 20 μl of the standard mixed with 2 μl of gold conjugate, chased by a further 20 μl of PBST. Strips were run for 10 minutes and cube readings recorded (Table 6 and FIG. 7).

TABLE 6
Initial testing of pairings in different orientations using a range of Fel d 1 concentrations.
Average cube units

| Pairing | Conjugation | NC | 0 μg/ml | 0.001 μg/ml | 0.01 μg/ml | 0.1 μg/ml | 1 μg/ml | 10 μg/ml |
|---|---|---|---|---|---|---|---|---|
| 1 | MAb | MAb | 1.9 | 25 | 102.5 | 198 | 188 | 157 |
| | 2 | 20 | | | | | | |
| 2 | MAb | MAb 2 | 4 | 35 | 108.5 | 187 | 195 | 143.5 |
| | 20 | | | | | | | |
| 3 | MAb | MAb | 1 | 3.6 | 8.4 | 86.5 | 163 | 112 |
| | 18 | 20 | | | | | | |
| 4 | MAb | MAb | 3.7 | 10 | 36 | 128.5 | 111.5 | 81 |
| | 20 | 18 | | | | | | |
| 5 | MAb | MAb 2 | 1.1 | 13 | 79.5 | 208.5 | 236.5 | 142 |
| | 9 | | | | | | | |
| 6 | MAb | MAb 9 | 2.3 | 7.5 | 45 | 194.5 | 221 | 97.5 |
| | 2 | | | | | | | |

The pairing with the highest sensitivity was mAb 20 gold conjugation with MAb 2 on the nitrocellulose membrane (Pairing 2), it had an average cube reading at 35 cube units at 0.001 μg/ml which is a visible test line. The pairing of MAb 2 conjugation with MAb 20 on the NC (Pairing 1) had an average cube reading of 25 which is a visible test line.

mAb 9 conjugation and mAb 2 on the NC (Pairing 5) had a signal with 0.001 μg/ml. The test line produced had cube units of 13. Pairings 4 and 6 had a sensitivity down to 0.01 μg/ml where there were visible test lines with average cube units of 36 and 45, respectively. Pairing 3 had the lowest sensitivity with a barely visible test line at 0.01 μg/ml, the cube reading only at 8.4 which is below the cut off point for positive results of 10 cube units. All of the pairings had a hook effect at ˜10 μg/ml Fel d 1, this gives a dynamic range of 0.001p g/ml to 0.1 g/ml. Pairings 1 and 2 were selected for further investigation.

### Example 6: Dry Assay Format

Pairings 1 and 2 were tested in a dry format. The mAb 2 and mAb 20 conjugate were sprayed at 3 different spray rates, 0.8 μl/mm, 0.4 μl/mm and 0.2 μl/mm onto 8951 conjugate pad (17 mm). mAb2 and mAb20 were plotted at 1 mg/ml onto CN140 NC. The conjugate, NC, sink pad and sample pad were laminated as described above and cut into 5 mm strips and placed into devices.

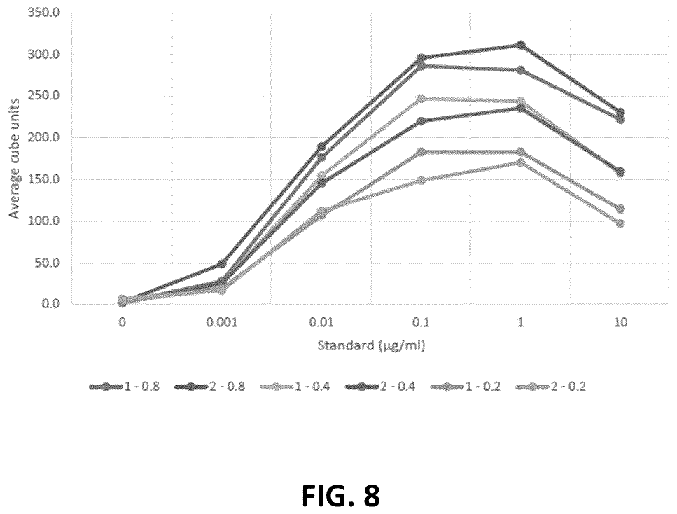

All three spray rates were tested with natural Fel d 1 antigen, which was first diluted to 10 μg/ml in PBST (0.1% Tween 20) and then subsequently from this a 10-fold dilution series was carried out until 0.001 μg/ml was reached. The devices were run in duplicate with 80 μl of each standard aliquoted into the well of the device. After ten minutes the device was read and the cube readings recorded (Table 7 and FIG. 8).

TABLE 7
Average cube units of pairing 1 and 2 with 3 different spray rates.

| | Average cube units | | | | | | |
|---|---|---|---|---|---|---|---|
| Spray | | 0 | 0.001 | 0.01 | 0.1 | 1 | 10 |
| rate | Pairing | μg/ml | μg/ml | μg/ml | μg/ml | ng/ml | μg/ml |
| 0.8 μl/mm | 1 | 2.3 | 28 | 176.5 | 288 | 281 | 222 |
| | 2 | 2.6 | 48.5 | 189.5 | 295.5 | 311 | 230.5 |
| 0.4 μl/mm | 1 | 2.3 | 25 | 154.5 | 247 | 243.5 | 157.5 |
| | 2 | 2.0 | 25.5 | 145.5 | 220 | 235.5 | 159.5 |
| 0.2 μl/mm | 1 | 3.3 | 19.5 | 106.5 | 183 | 183 | 114.5 |
| | 2 | 6.6 | 17 | 112 | 149 | 170.5 | 97 |

Results demonstrate that the ‘dry’ format is just as sensitive as the ‘wet’ format. There is a visible signal with a dilution of 0.001 μg/ml. Pairing 2 was more sensitive than pairing 1 with a cube reading of 48.5 with a 0.001 μg/ml dilution compared to 28. As the spray rate decreased so did the signal seen. Although there was still a visible signal with a 0.001 μg/ml dilution with the spray rate 0.2 μl/mm. There was still a visible hook effect with a 10 μg/ml dilution giving a dynamic range of 0.1 μg/ml to 0.001 μg/ml. There was no non-specific binding (NSB) seen with either pairing with any of the spray rates. A spray rate of 0.8 μL/mm for both pairings was chosen to test with a wider range of dilutions to form a dilution curve.

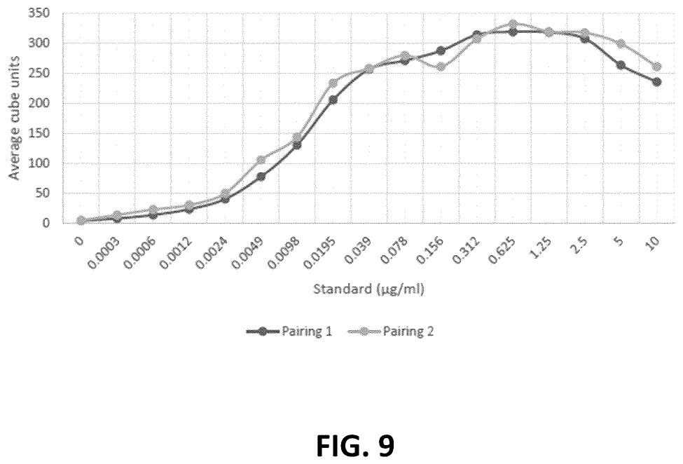

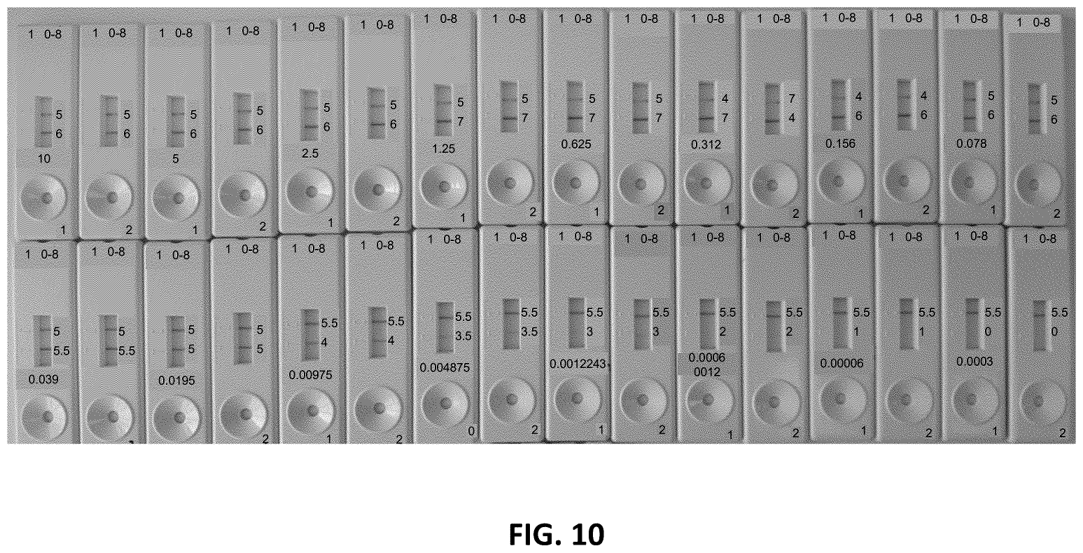

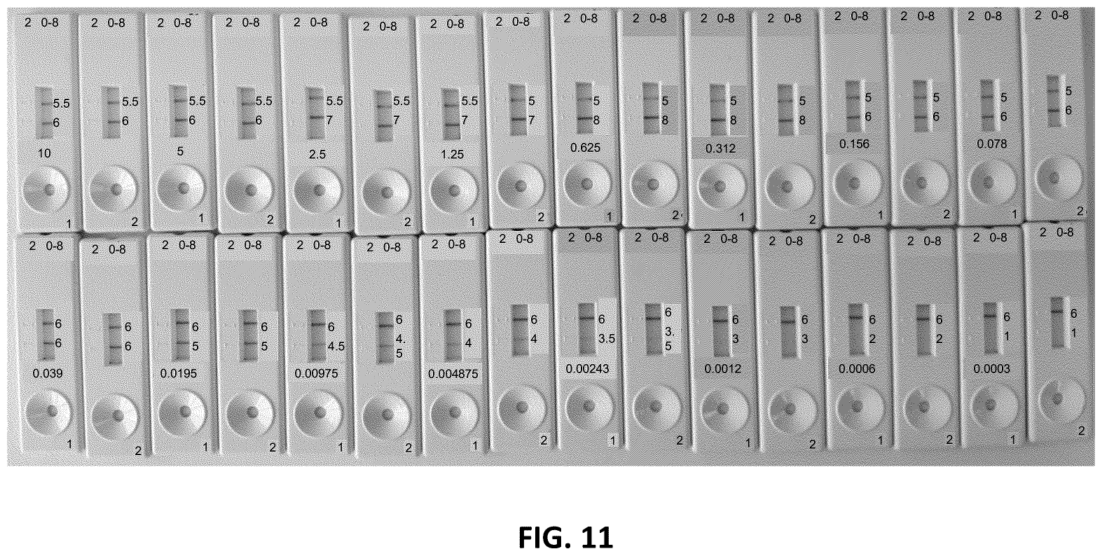

Only the spray rate 0.8 μl/mm was tested with Fel d 1 antigen which was first diluted to 10 μg/ml in PBST (0.1% Tween 20) and then subsequently from this, a 2-fold dilution series was carried out until 0.0003 μg/ml was reached. The devices were run in duplicate with 80 μl of each standard aliquoted into the well of the device. After ten minutes the device was read and the cube readings recorded (Table 8 and FIG. 9). Images of initial dry testing of pairing 1 (conjugate mAb2 and NC Mab20) and (conjugate mAb20 and NC mAb2) are shown in FIG. 10 and FIG. 11, respectively.

TABLE 8
Average cube units of pairing 1 and 2 tested with doubling dilution series.
Average cube units

| Standard | Pairing 1 | Pairing 2 |
|---|---|---|
| 0 | 3.2 | 4 |
| 0.0003 | 7.1 | 13 |
| 0.0006 | 13 | 22 |
| 0.0012 | 22.5 | 29.5 |
| 0.0024 | 39.5 | 49 |
| 0.0049 | 77 | 105 |
| 0.0098 | 130 | 143.5 |
| 0.0195 | 205 | 233 |
| 0.039 | 256 | 257.5 |
| 0.078 | 270.5 | 279 |
| 0.156 | 287 | 260.5 |
| 0.312 | 313.5 | 306.5 |
| 0.625 | 318.5 | 331.5 |
| 1.25 | 317.5 | 319 |
| 2.5 | 307 | 317 |
| 5 | 263 | 298.5 |
| 10 | 235 | 260.5 |

Pairing 2 was determined to be marginally more sensitive than pairing 1, it still gives a visible test line and a cube unit of 13 at 0.0003 μg/ml Fel d 1. The hook effect could be seen clearly with both pairings and gives a dynamic range of approximately 0.625 μg/ml-0.0003 μg/ml (625 ng/ml-0.3 ng/ml). Neither of the pairings had any visible NSB and the cube readings were below the 10 cube unit cut off point, at 3.2 for pairing 1 and 4 for pairing 2. Pairing 2 had good sensitivity with no NSB.

TABLE 9
Heavy Chain Variable Domain (VH) and Heavy Chain Constant Region amino acid sequences

| | | No | No | | | | | |
|---|---|---|---|---|---|---|---|---|
| cAb | Clone | of | of | HC | | Signal | | |
| ID | Name | HCs | LCs | Type | Integrity | peptide | VH | Heavy Chain Constant Region |
| cAb10204 | 2291.014.055. | 1 | 1 | Mouse | Complete | MNFGLSLI | EVKLMESGGGLVKP | AKTTPPSVYPLAPGSAAQTNSM |
| | 067 | | | IgG1 | | FLVLILKG | GGSLKLSCAASGFTF | VTLGCLVKGYFPEPVTVTWNS |
| | (fel d1 MAb2) | | | | | VQC (SEQ | SSYDMSWVRQTPEK | GSLSSGVHTFPAVLQSDLYTLS |
| | | | | | | ID NO: 1) | RLEWVATISSGGTY | SSVTVPSSTWPSETVTCNVAHP |
| | | | | | | | TYYPDSLKGRFTISR | ASSTKVDKKIVPRDCGCKPCIC |
| | | | | | | | DNARNTLYLQMSSL | TVPEVSSVFIFPPKPKDVLTITLT |
| | | | | | | | RSEDTALFYCARLL | PKVTCVVVDISKDDPEVQFSWF |
| | | | | | | | RTYAMDYWGQGTS | VDDVEVHTAQTQPREEQFNSTF |
| | | | | | | | VTVSS (SEQ ID NO: | RSVSELPIMHQDWLNGKEFKC |
| | | | | | | | 10) | RVNSAAFPAPIEKTISKTKGRPK |
| | | | | | | | | APQVYTIPPPKEQMAKDKVSLT |
| | | | | | | | | CMITDFFPEDITVEWQWNGQPA |
| | | | | | | | | ENYKNTQPIMDTDGSYFVYSKL |
| | | | | | | | | NVQKSNWEAGNTFTCSVLHEG |
| | | | | | | | | LHNHHTEKSLSHSPGK (SEQ ID |
| | | | | | | | | NO: 19) |
| cAb10205 | 2293.254.030 | 1 | 1 | Mouse | Complete | MGWSYIIL | QVQLQQPGAELVKP | AKTTPPSVYPLAPGSAAQTNSM |
| | (Fel d1 MAb9) | | | IgG1 | | FLVATAT | GASVKLSCKASGYT | VTLGCLVKGYFPEPVTVTWNS |
| | | | | | | DVHS | FTSYWMYWVKQRP | GSLSSGVHTFPAVLQSDLYTLS |
| | | | | | | (SEQ ID | GQGLEWIGEINPSNG | SSVTVPSSTWPSETVTCNVAHP |
| | | | | | | NO: 2) | RTNYNEKFKTKATL | ASSTKVDKKIVPRDCGCKPCIC |
| | | | | | | | TVDKSSSTAYMQLS | TVPEVSSVFIFPPKPKDVLTITLT |
| | | | | | | | SLTSEDSAVYYCAR | PKVTCVVVDISKDDPEVQFSWF |
| | | | | | | | RATMIGGFAYWGQ | VDDVEVHTAQTQPREEQFNSTF |
| | | | | | | | GTLVTVSA (SEQ ID | RSVSELPIMHQDWLNGKEFKC |
| | | | | | | | NO: 11) | RVNSAAFPAPIEKTISKTKGRPK |
| | | | | | | | | APQVYTIPPPKEQMAKDKVSLT |
| | | | | | | | | CMITDFFPEDITVEWQWNGQPA |
| | | | | | | | | ENYKNTQPIMDTDGSYFVYSKL |
| | | | | | | | | NVQKSNWEAGNTFTCSVLHEG |
| | | | | | | | | LHNHHTEKSLSHSPGK (SEQ ID |
| | | | | | | | | NO: 19) |
| cAb10206 | 2297.167. | 1 | 1 | Mouse | Complete | MEWNWV | QGQMQQSGAELVK | AKTTPPSVYPLAPGSAAQTNSM |
| | 064 | | | IgG1 | | VLFLLSLT | PGASVKLSCKTSGFT | VTLGCLVKGYFPEPVTVTWNS |
| | (Fel d1 MAb18) | | | | | AGVYA | FSSSYISWLKQKPGQ | GSLSSGVHTFPAVLQSDLYTLS |
| | | | | | | (SEQ ID | SLEWIAWIYAGTGG | SSVTVPSSTWPSETVTCNVAHP |
| | | | | | | NO: 3) | TTYNQRFTGKAQLT | ASSTKVDKKIVPRDCGCKPCIC |
| | | | | | | | VDSSSSTAYMHFSSL | TVPEVSSVFIFPPKPKDVLTITLT |
| | | | | | | | TTDDSAIYYCARHY | PKVTCVVVDISKDDPEVQFSWF |
| | | | | | | | GNPPAWFAYWGQG | VDDVEVHTAQTQPREEQFNSTF |
| | | | | | | | TLVTVSA (SEQ ID | RSVSELPIMHQDWLNGKEFKC |
| | | | | | | | NO: 12) | RVNSAAFPAPIEKTISKTKGRPK |
| | | | | | | | | APQVYTIPPPKEQMAKDKVSLT |
| | | | | | | | | CMITDFFPEDITVEWQWNGQPA |
| | | | | | | | | ENYKNTQPIMDTDGSYFVYSKL |
| | | | | | | | | NVQKSNWEAGNTFTCSVLHEG |
| | | | | | | | | LHNHHTEKSLSHSPGK (SEQ ID |
| | | | | | | | | NO: 19) |
| cAb10207 | 2297.179. | 2 | 1 | Mouse | Complete | MGWSCIIL | QVQLQQPGTELVRP | AKTTPPSVYPLAPGSAAQTNSM |
| | 089 | | | IgG1 | | FLVATAT | GASVKLSCKASGYT | VTLGCLVKGYFPEPVTVTWNS |
| | (Fel d1 MAb20) | | | | | GVHS | FTSYWINWVKQRPG | GSLSSGVHTFPAVLQSDLYTLS |
| | | | | | | (SEQ ID | QGLEWIGNIYPSDSY | SSVTVPSSTWPSETVTCNVAHP |
| | | | | | | NO: 4) | TNYNQKFKDKATLT | ASSTKVDKKIVPRDCGCKPCIC |
| | | | | | | | VDKSSSTAYMQLSS | TVPEVSSVFIFPPKPKDVLTITLT |
| | | | | | | | PTSDDSAVYYCTRR | PKVTCVVVDISKDDPEVQFSWF |
| | | | | | | | GNYGNLYYFDYWG | VDDVEVHTAQTQPREEQFNSTF |
| | | | | | | | QGTTLTVSS (SEQ ID | RSVSELPIMHQDWLNGKEFKC |
| | | | | | | | NO: 13) | RVNSAAFPAPIEKTISKTKGRPK |
| | | | | | | | | APQVYTIPPPKEQMAKDKVSLT |
| | | | | | | | | CMITDFFPEDITVEWQWNGQPA |
| | | | | | | | | ENYKNTQPIMDTDGSYFVYSKL |
| | | | | | | | | NVQKSNWEAGNTFTCSVLHEG |
| | | | | | | | | LHNHHTEKSLSHSPGK (SEQ ID |
| | | | | | | | | NO: 19) |
| cAb10207 | 2297.179. | 2 | 1 | — | Partial | MDWLWN | QIQLVQSGPELRKPG | AKTTPPSVYPLAPGSAAQTNSM |
| | 089 | | | | | LLFLMAA | ETVRISCKASGYTFT | VTLGCLV (SEQ ID NO: 20) |
| | (Fel d1- | | | | | AQSIQA | TYGMDWVKQAPGK | |
| | MAb201) | | | | | (SEQ ID | VLKWVGWINTNTG | |
| | | | | | | NO: 5) | EPTYAEEFKGRFAFS | |
| | | | | | | | LETSASTAYFQINNL | |
| | | | | | | | KNEDTATYFCARK | |
| | | | | | | | WLRRAMDYWGQG | |
| | | | | | | | TSVTVSS (SEQ ID | |
| | | | | | | | NO: 14) | |

TABLE 10
Light Chain Variable Domain (VL) and Light Chain Constant Region amino acid sequences

| | | No | No | | | | | |
|---|---|---|---|---|---|---|---|---|
| cAb | Clone | of | of | LC | In- | Signal | | Light Chain |
| ID | Name | HCs | LCs | Type | tegrity | peptide | VL | Constant Region |
| cAb1 | 2291.014. | 1 | 1 | Mouse | Complete | MRVLAE | DIQMNQSPSSLSASL | RADAAPTVSIFPPSSEQLTSGGA |
| 0204 | 055.067 | | | kappa | | LLGLLLF | GDTITITCHASQNIIV | SVVCFLNNFYPKDINVKWKIDG |
| | (fel d1 | | | | | CFLGVR | WLNWYQQKPGNIP | SERQNGVLNSWTDQDSKDSTY |
| | MAb2) | | | | | C (SEQ | KLLIYKASNLHTGV | SMSSTLTLTKDEYERHNSYTCE |
| | | | | | | ID | PSRFSGSGSGTGFTL | ATHKTSTSPIVKSFNRNEC |
| | | | | | | NO: 6) | TISSLQPEDIATYYC | (SEQ ID NO: 21) |
| | | | | | | | QQGQSYHTWTFGG | |
| | | | | | | | GTKLEIK (SEQ ID | |
| | | | | | | | NO: 15) | |
| cAb1 | 2293.254. | 1 | 1 | Mouse | Complete | MESQTQ | DIVMTQSPSSLTVTT | RADAAPTVSIFPPSSEQLTSGGA |
| 0205 | 030 | | | kappa | | VLMSLL | GEKVTMNCKSSQSL | SVVCFLNNFYPKDINVKWKIDG |
| | (Fel d1 | | | | | FWVSGT | LNSANQKNYLTWH | SERQNGVLNSWTDQDSKDSTY |
| | MAb9) | | | | | CG (SEQ | QQKPGQPPKLLIYW | SMSSTLTLTKDEYERHNSYTCE |
| | | | | | | ID | ASTRESGVPDRFTGS | ATHKTSTSPIVKSFNRNEC |
| | | | | | | NO: 7) | GSGTDFTLTISSVQA | (SEQ ID NO: 21) |
| | | | | | | | EDLAVYYCONDYSF | |
| | | | | | | | PFTFGSGTKLEIK | |
| | | | | | | | (SEQ ID NO: 16) | |
| cAb1 | 2297.167. | 1 | 1 | Mouse | Complete | MSVLTQ | DIQMTQSPASLSASV | RADAAPTVSIFPPSSEQLTSGGA |
| 0206 | 064 | | | kappa | | VLALLL | GETVTITCRASGNIH | SVVCFLNNFYPKDINVKWKIDG |
| | (Fel d1 | | | | | LWLTGA | NYLAWFQQRQGKS | SERQNGVLNSWTDQDSKDSTY |
| | MAb18) | | | | | RC (SEQ | PQLLVYNAETLADG | SMSSTLTLTKDEYERHNSYTCE |
| | | | | | | ID | VPSRFSGSGSGTQYS | ATHKTSTSPIVKSFNRNEC |
| | | | | | | NO: 8) | LKISSLQPEDFGSYF | (SEQ ID NO: 21) |
| | | | | | | | CQHFWSRPPTFGGG | |
| | | | | | | | TKLEIK (SEQ ID | |
| | | | | | | | NO: 17) | |
| cAb1 | 2297.179. | 2 | 1 | Mouse | Complete | MESQTL | NIVMTQSPKSMSMS | RADAAPTVSIFPPSSEQLTSGGA |
| 0207 | 089 | | | kappa | | VFISILL | VGERVTLSCKASEN | SVVCFLNNFYPKDINVKWKIDG |
| | (Fel d1 | | | | | WLYGA | VGSYVFWYQQKPD | SERQNGVLNSWTDQDSKDSTY |
| | MAb20) | | | | | DG (SEQ | QSPKLLIYGASNRYT | SMSSTLTLTKDEYERHNSYTCE |
| | | | | | | ID | GVPDRFTGSRSATD | ATHKTSTSPIVKSFNRNEC |
| | | | | | | NO: 9) | FTLTISNVQAEDLAD | (SEQ ID NO: 21) |
| | | | | | | | YHCGQSYSYPLTFG | |
| | | | | | | | AGTKLELK (SEQ ID | |
| | | | | | | | NO: 18) | |

TABLE 11
Examples of polynucleotide sequences encoding Heavy Chain Variable Domain (VH) and Heavy Chain
Constant Region

| | | No | No | | | | | |
|---|---|---|---|---|---|---|---|---|
| cAb | Clone | of | of | HC | In- | Signal | | Heavy Chain |
| ID | Name | HCs | LCs | Type | tegrity | peptide | VH | Constant Region |
| cAb1 | 2291.014. | 1 | 1 | Mouse | Complete | ATGAAC | GAGGTGAAGCTGA | GCCAAAACGACACCCCCATCT |
| 0204 | 055.067 | | | IgG1 | | TTTGGG | TGGAGTCTGGGGG | GTCTATCCACTGGCCCCTGGA |
| | (fel d1 | | | | | CTGAGC | AGGCTTAGTGAAG | TCTGCTGCCCAAACTAACTCC |
| | MAb2) | | | | | TTGATT | CCTGGAGGGTCCCT | ATGGTGACCCTGGGATGCCTG |
| | | | | | | TTCCTT | GAAACTCTCCTGTG | GTCAAGGGCTATTTCCCTGAG |
| | | | | | | GTCCTA | CAGCCTCTGGATTC | CCAGTGACAGTGACCTGGAAC |
| | | | | | | ATTTTA | ACTTTCAGTAGTTA | TCTGGATCCCTGTCCAGCGGT |
| | | | | | | AAAGGT | TGACATGTCTTGGG | GTGCACACCTTCCCAGCTGTC |
| | | | | | | GTCCAG | TTCGCCAGACTCCG | CTGCAGTCTGACCTCTACACT |
| | | | | | | TGT | GAGAAGAGGCTGG | CTGAGCAGCTCAGTGACTGTC |
| | | | | | | (SEQ ID | AGTGGGTCGCAAC | CCCTCCAGCACCTGGCCCAGC |
| | | | | | | NO: 22) | CATTAGTAGTGGTG | GAGACCGTCACCTGCAACGTT |
| | | | | | | | GTACTTACACCTAC | GCCCACCCGGCCAGCAGCACC |
| | | | | | | | TATCCAGACAGTCT | AAGGTGGACAAGAAAATTGT |
| | | | | | | | AAAGGGCCGATTC | GCCCAGGGATTGTGGTTGTAA |
| | | | | | | | ACCATCTCCAGAGA | GCCTTGCATATGTACAGTCCC |
| | | | | | | | CAATGCCAGGAAC | AGAAGTATCATCTGTCTTCAT |
| | | | | | | | ACCCTGTACCTGCA | CTTCCCCCCAAAGCCCAAGGA |
| | | | | | | | AATGAGCAGTCTG | TGTGCTCACCATTACTCTGAC |
| | | | | | | | AGGTCTGAGGACA | TCCTAAGGTCACGTGTGTTGT |
| | | | | | | | CGGCCTTGTTTTAC | GGTAGACATCAGCAAGGATG |
| | | | | | | | TGTGCAAGACTACT | ATCCCGAGGTCCAGTTCAGCT |
| | | | | | | | TCGGACCTATGCTA | GGTTTGTAGATGATGTGGAGG |
| | | | | | | | TGGACTACTGGGGT | TGCACACAGCTCAGACGCAAC |
| | | | | | | | CAAGGAACCTCAG | CCCGGGAGGAGCAGTTCAAC |
| | | | | | | | TCACCGTCTCCTCA | AGCACTTTCCGCTCAGTCAGT |
| | | | | | | | (SEQ ID NO: 31) | GAACTTCCCATCATGCACCAG |
| | | | | | | | | GACTGGCTCAATGGCAAGGA |
| | | | | | | | | GTTCAAATGCAGGGTCAACAG |
| | | | | | | | | TGCAGCTTTCCCTGCCCCCAT |
| | | | | | | | | CGAGAAAACCATCTCCAAAAC |
| | | | | | | | | CAAAGGCAGACCGAAGGCTC |
| | | | | | | | | CACAGGTGTACACCATTCCAC |
| | | | | | | | | CTCCCAAGGAGCAGATGGCCA |
| | | | | | | | | AGGATAAAGTCAGTCTGACCT |
| | | | | | | | | GCATGATAACAGACTTCTTCC |
| | | | | | | | | CTGAAGACATTACTGTGGAGT |
| | | | | | | | | GGCAGTGGAATGGGCAGCCA |
| | | | | | | | | GCGGAGAACTACAAGAACAC |
| | | | | | | | | TCAGCCCATCATGGACACAGA |
| | | | | | | | | TGGCTCTTACTTCGTCTACAG |
| | | | | | | | | CAAGCTCAATGTGCAGAAGA |
| | | | | | | | | GCAACTGGGAGGCAGGAAAT |
| | | | | | | | | ACTTTCACCTGCTCTGTGTTAC |
| | | | | | | | | ATGAGGGCCTGCACAACCACC |
| | | | | | | | | ATACTGAGAAGAGCCTCTCCC |
| | | | | | | | | ACTCTCCTGGTAAA (SEQ ID |
| | | | | | | | | NO: 66) |
| cAb1 | 2293.254. | 1 | 1 | Mouse | Complete | ATGGGA | CAGGTCCAACTGCA | GCCAAAACGACACCCCCATCT |
| 0205 | 030 | | | IgG1 | | TGGAGC | GCAGCCTGGGGCT | GTCTATCCACTGGCCCCTGGA |
| | (Fel d1 | | | | | TATATC | GAACTGGTGAAGC | TCTGCTGCCCAAACTAACTCC |
| | MAb9) | | | | | ATCCTC | CTGGGGCTTCAGTG | ATGGTGACCCTGGGATGCCTG |
| | | | | | | TTTTTG | AAGCTGTCCTGCAA | GTCAAGGGCTATTTCCCTGAG |
| | | | | | | GTAGCA | GGCTTCTGGCTACA | CCAGTGACAGTGACCTGGAAC |
| | | | | | | ACAGCT | CCTTCACCAGCTAC | TCTGGATCCCTGTCCAGCGGT |
| | | | | | | ACAGAT | TGGATGTACTGGGT | GTGCACACCTTCCCAGCTGTC |
| | | | | | | GTCCAC | AAAACAGAGGCCT | CTGCAGTCTGACCTCTACACT |
| | | | | | | TCC | GGACAAGGCCTTG | CTGAGCAGCTCAGTGACTGTC |
| | | | | | | (SEQ ID | AGTGGATTGGAGA | CCCTCCAGCACCTGGCCCAGC |
| | | | | | | NO: 23) | GATTAATCCTAGCA | GAGACCGTCACCTGCAACGTT |
| | | | | | | | ACGGTCGTACTAAC | GCCCACCCGGCCAGCAGCACC |
| | | | | | | | TACAATGAGAAGTT | AAGGTGGACAAGAAAATTGT |
| | | | | | | | CAAGACCAAGGCC | GCCCAGGGATTGTGGTTGTAA |
| | | | | | | | ACACTGACTGTAGA | GCCTTGCATATGTACAGTCCC |
| | | | | | | | CAAATCCTCCAGCA | AGAAGTATCATCTGTCTTCAT |
| | | | | | | | CAGCCTACATGCAA | CTTCCCCCCAAAGCCCAAGGA |
| | | | | | | | CTCAGCAGCCTGAC | TGTGCTCACCATTACTCTGAC |
| | | | | | | | ATCTGAGGACTCTG | TCCTAAGGTCACGTGTGTTGT |
| | | | | | | | CGGTCTATTACTGT | GGTAGACATCAGCAAGGATG |
| | | | | | | | GCAAGACGAGCTA | ATCCCGAGGTCCAGTTCAGCT |
| | | | | | | | CTATGATTGGGGGG | GGTTTGTAGATGATGTGGAGG |
| | | | | | | | TTTGCTTACTGGGG | TGCACACAGCTCAGACGCAAC |
| | | | | | | | CCAAGGGACTCTG | CCCGGGAGGAGCAGTTCAAC |
| | | | | | | | GTCACTGTCTCTGC | AGCACTTTCCGCTCAGTCAGT |
| | | | | | | | A (SEQ ID | GAACTTCCCATCATGCACCAG |
| | | | | | | | NO: 32) | GACTGGCTCAATGGCAAGGA |
| | | | | | | | | GTTCAAATGCAGGGTCAACAG |
| | | | | | | | | TGCAGCTTTCCCTGCCCCCAT |
| | | | | | | | | CGAGAAAACCATCTCCAAAAC |
| | | | | | | | | CAAAGGCAGACCGAAGGCTC |
| | | | | | | | | CACAGGTGTACACCATTCCAC |
| | | | | | | | | CTCCCAAGGAGCAGATGGCCA |
| | | | | | | | | AGGATAAAGTCAGTCTGACCT |
| | | | | | | | | GCATGATAACAGACTTCTTCC |
| | | | | | | | | CTGAAGACATTACTGTGGAGT |
| | | | | | | | | GGCAGTGGAATGGGCAGCCA |
| | | | | | | | | GCGGAGAACTACAAGAACAC |
| | | | | | | | | TCAGCCCATCATGGACACAGA |
| | | | | | | | | TGGCTCTTACTTCGTCTACAG |
| | | | | | | | | CAAGCTCAATGTGCAGAAGA |
| | | | | | | | | GCAACTGGGAGGCAGGAAAT |
| | | | | | | | | ACTTTCACCTGCTCTGTGTTAC |
| | | | | | | | | ATGAGGGCCTGCACAACCACC |
| | | | | | | | | ATACTGAGAAGAGCCTCTCCC |
| | | | | | | | | ACTCTCCTGGTAAA (SEQ ID |
| | | | | | | | | NO: 66) |
| cAb1 | 2297.167. | 1 | 1 | Mouse | Complete | ATGGAA | CAGGGTCAGATGC | GCCAAAACGACACCCCCATCT |
| 0206 | 064 | | | IgG1 | | TGGAAC | AGCAGTCTGGAGCT | GTCTATCCACTGGCCCCTGGA |
| | (Fel d1 | | | | | TGGGTC | GAGCTGGTGAAGC | TCTGCTGCCCAAACTAACTCC |
| | MAb18) | | | | | GTTCTC | CTGGGGCTTCAGTG | ATGGTGACCCTGGGATGCCTG |
| | | | | | | TTCCTC | AAACTGTCCTGCAA | GTCAAGGGCTATTTCCCTGAG |
| | | | | | | CTGTCA | GACTTCTGGCTTCA | CCAGTGACAGTGACCTGGAAC |
| | | | | | | TTAACT | CCTTCAGCAGTAGT | TCTGGATCCCTGTCCAGCGGT |
| | | | | | | GCAGGT | TATATAAGTTGGTT | GTGCACACCTTCCCAGCTGTC |
| | | | | | | GTCTAT | GAAGCAAAAGCCT | CTGCAGTCTGACCTCTACACT |
| | | | | | | GCC | GGACAGAGTCTTG | CTGAGCAGCTCAGTGACTGTC |
| | | | | | | (SEQ ID | AGTGGATTGCATGG | CCCTCCAGCACCTGGCCCAGC |
| | | | | | | NO: 24) | ATTTATGCTGGAAC | GAGACCGTCACCTGCAACGTT |
| | | | | | | | TGGTGGAACTACCT | GCCCACCCGGCCAGCAGCACC |
| | | | | | | | ATAATCAGAGGTTC | AAGGTGGACAAGAAAATTGT |
| | | | | | | | ACAGGCAAGGCCC | GCCCAGGGATTGTGGTTGTAA |
| | | | | | | | AACTGACTGTAGAC | GCCTTGCATATGTACAGTCCC |
| | | | | | | | TCATCCTCCAGCAC | AGAAGTATCATCTGTCTTCAT |
| | | | | | | | AGCCTACATGCATT | CTTCCCCCCAAAGCCCAAGGA |
| | | | | | | | TCAGCAGCCTGACA | TGTGCTCACCATTACTCTGAC |
| | | | | | | | ACTGATGACTCTGC | TCCTAAGGTCACGTGTGTTGT |
| | | | | | | | CATCTATTACTGTG | GGTAGACATCAGCAAGGATG |
| | | | | | | | CAAGACACTATGGT | ATCCCGAGGTCCAGTTCAGCT |
| | | | | | | | AACCCCCCCGCCTG | GGTTTGTAGATGATGTGGAGG |
| | | | | | | | GTTTGCTTACTGGG | TGCACACAGCTCAGACGCAAC |
| | | | | | | | GCCAAGGGACTCT | CCCGGGAGGAGCAGTTCAAC |
| | | | | | | | GGTCACTGTCTCTG | AGCACTTTCCGCTCAGTCAGT |
| | | | | | | | CA (SEQ ID | GAACTTCCCATCATGCACCAG |
| | | | | | | | NO: 33) | GACTGGCTCAATGGCAAGGA |
| | | | | | | | | GTTCAAATGCAGGGTCAACAG |
| | | | | | | | | TGCAGCTTTCCCTGCCCCCAT |
| | | | | | | | | CGAGAAAACCATCTCCAAAAC |
| | | | | | | | | CAAAGGCAGACCGAAGGCTC |
| | | | | | | | | CACAGGTGTACACCATTCCAC |
| | | | | | | | | CTCCCAAGGAGCAGATGGCCA |
| | | | | | | | | AGGATAAAGTCAGTCTGACCT |
| | | | | | | | | GCATGATAACAGACTTCTTCC |
| | | | | | | | | CTGAAGACATTACTGTGGAGT |
| | | | | | | | | GGCAGTGGAATGGGCAGCCA |
| | | | | | | | | GCGGAGAACTACAAGAACAC |
| | | | | | | | | TCAGCCCATCATGGACACAGA |
| | | | | | | | | TGGCTCTTACTTCGTCTACAG |
| | | | | | | | | CAAGCTCAATGTGCAGAAGA |
| | | | | | | | | GCAACTGGGAGGCAGGAAAT |
| | | | | | | | | ACTTTCACCTGCTCTGTGTTAC |
| | | | | | | | | ATGAGGGCCTGCACAACCACC |
| | | | | | | | | ATACTGAGAAGAGCCTCTCCC |
| | | | | | | | | ACTCTCCTGGTAAA (SEQ ID |
| | | | | | | | | NO: 66) |
| cAb1 | 2297.179. | 2 | 1 | Mouse | Complete | ATGGGA | CAGGTCCAACTGCA | GCCAAAACGACACCCCCATCT |
| 0207 | 089 | | | IgG1 | | TGGAGC | GCAGCCTGGGACT | GTCTATCCACTGGCCCCTGGA |
| | (Fel d1 | | | | | TGTATC | GAGTTGGTGAGGC | TCTGCTGCCCAAACTAACTCC |
| | MAb20) | | | | | ATCCTC | CTGGGGCTTCAGTG | ATGGTGACCCTGGGATGCCTG |
| | | | | | | TTCTTG | AAGCTGTCCTGCAA | GTCAAGGGCTATTTCCCTGAG |
| | | | | | | GTAGCA | GGCTTCTGGCTACA | CCAGTGACAGTGACCTGGAAC |
| | | | | | | ACAGCT | CCTTCACCAGCTAC | TCTGGATCCCTGTCCAGCGGT |
| | | | | | | ACAGGT | TGGATAAACTGGGT | GTGCACACCTTCCCAGCTGTC |
| | | | | | | GTCCAC | GAAGCAGAGGCCT | CTGCAGTCTGACCTCTACACT |
| | | | | | | TCC | GGACAAGGCCTTG | CTGAGCAGCTCAGTGACTGTC |
| | | | | | | (SEQ ID | AGTGGATCGGAAA | CCCTCCAGCACCTGGCCCAGC |
| | | | | | | NO: 25) | TATTTATCCTTCTG | GAGACCGTCACCTGCAACGTT |
| | | | | | | | ATAGTTATACTAAC | GCCCACCCGGCCAGCAGCACC |
| | | | | | | | TACAATCAAAAGTT | AAGGTGGACAAGAAAATTGT |
| | | | | | | | CAAGGACAAGGCC | GCCCAGGGATTGTGGTTGTAA |
| | | | | | | | ACATTGACTGTAGA | GCCTTGCATATGTACAGTCCC |
| | | | | | | | CAAATCCTCCAGTA | AGAAGTATCATCTGTCTTCAT |
| | | | | | | | CAGCCTACATGCAG | CTTCCCCCCAAAGCCCAAGGA |
| | | | | | | | CTCAGCAGCCCGAC | TGTGCTCACCATTACTCTGAC |
| | | | | | | | ATCTGACGACTCTG | TCCTAAGGTCACGTGTGTTGT |
| | | | | | | | CGGTCTATTACTGT | GGTAGACATCAGCAAGGATG |
| | | | | | | | ACAAGAAGGGGCA | ATCCCGAGGTCCAGTTCAGCT |
| | | | | | | | ACTATGGTAACCTA | GGTTTGTAGATGATGTGGAGG |
| | | | | | | | TACTACTTTGACTA | TGCACACAGCTCAGACGCAAC |
| | | | | | | | CTGGGGCCAAGGC | CCCGGGAGGAGCAGTTCAAC |
| | | | | | | | ACCACTCTCACAGT | AGCACTTTCCGCTCAGTCAGT |
| | | | | | | | CTCCTCA (SE | GAACTTCCCATCATGCACCAG |
| | | | | | | | ID NO: 34) | GACTGGCTCAATGGCAAGGA |
| | | | | | | | | GTTCAAATGCAGGGTCAACAG |
| | | | | | | | | TGCAGCTTTCCCTGCCCCCAT |
| | | | | | | | | CGAGAAAACCATCTCCAAAAC |
| | | | | | | | | CAAAGGCAGACCGAAGGCTC |
| | | | | | | | | CACAGGTGTACACCATTCCAC |
| | | | | | | | | CTCCCAAGGAGCAGATGGCCA |
| | | | | | | | | AGGATAAAGTCAGTCTGACCT |
| | | | | | | | | GCATGATAACAGACTTCTTCC |
| | | | | | | | | CTGAAGACATTACTGTGGAGT |
| | | | | | | | | GGCAGTGGAATGGGCAGCCA |
| | | | | | | | | GCGGAGAACTACAAGAACAC |
| | | | | | | | | TCAGCCCATCATGGACACAGA |
| | | | | | | | | TGGCTCTTACTTCGTCTACAG |
| | | | | | | | | CAAGCTCAATGTGCAGAAGA |
| | | | | | | | | GCAACTGGGAGGCAGGAAAT |
| | | | | | | | | ACTTTCACCTGCTCTGTGTTAC |
| | | | | | | | | ATGAGGGCCTGCACAACCACC |
| | | | | | | | | ATACTGAGAAGAGCCTCTCCC |
| | | | | | | | | ACTCTCCTGGTAAA (SEQ ID |
| | | | | | | | | NO: 66) |
| cAb1 | 2297.179. | 2 | 1 | — | Partial | ATGGAT | CAGATCCAGTTGGT | GCCAAAACGACACCCCCATCT |
| 0207 | 089 | | | | | TGGCTG | GCAGTCTGGACCTG | GTCTATCCACTGGCCCCTGGA |
| | (Fel d1 | | | | | TGGAAC | AGCTGAGGAAGCC | TCTGCTGCCCAAACTAACTCC |
| | MAb20) | | | | | TTGCTA | TGGAGAGACAGTC | ATGGTGACCCTGGGATGCCTG |
| | | | | | | TTCCTG | AGGATCTCCTGCAA | GTC (SEQ ID NO: 40) |
| | | | | | | ATGGCA | GGCTTCTGGGTATA | |
| | | | | | | GCTGCC | CCTTCACAACCTAT | |
| | | | | | | CAAAGT | GGAATGGACTGGG | |
| | | | | | | ATCCAA | TGAAGCAGGCTCC | |
| | | | | | | GCA | AGGAAAGGTTTTA | |
| | | | | | | (SEQ ID | AAGTGGGTGGGCT | |
| | | | | | | NO: 26) | GGATAAACACCAA | |
| | | | | | | | CACTGGAGAGCCA | |
| | | | | | | | ACATATGCTGAAG | |
| | | | | | | | AGTTCAAGGGACG | |
| | | | | | | | GTTTGCCTTCTCTTT | |
| | | | | | | | GGAAACCTCTGCCA | |
| | | | | | | | GCACTGCCTATTTC | |
| | | | | | | | CAGATCAACAACCT | |
| | | | | | | | CAAAAATGAGGAC | |
| | | | | | | | ACGGCTACATATTT | |
| | | | | | | | CTGTGCAAGAAAA | |
| | | | | | | | TGGTTACGACGGGC | |
| | | | | | | | TATGGACTACTGGG | |
| | | | | | | | GTCAAGGAACCTC | |
| | | | | | | | AGTCACCGTCTCCT | |
| | | | | | | | CA (SEQ ID | |
| | | | | | | | NO: 35) | |

TABLE 12
Examples of polynucleotide sequences encoding Light Chain Variable Domain (VL) and Light
Chain Constant Region

| | | No | No | | | | | |
|---|---|---|---|---|---|---|---|---|
| cAb | Clone | of | of | LC | In- | Signal | | Light Chain |
| ID | Name | HCs | LCs | Type | tegrity | peptide | VL | Constant Region |
| cAb1 | 2291.014. | 1 | 1 | Mouse | Complete | ATGAGG | GACATCCAGATGAA | CGGGCTGATGCTGCACCAAC |
| 0204 | 055.067 | | | kappa | | GTCCTT | CCAGTCTCCATCCA | TGTATCCATCTTCCCACCAT |
| | (fel d1 | | | | | GCTGAG | GTCTGTCTGCATCCC | CCAGTGAGCAGTTAACATCT |
| | MAb2) | | | | | CTCCTG | TTGGAGACACAATT | GGAGGTGCCTCAGTCGTGTG |
| | | | | | | GGGCTG | ACCATCACTTGCCAT | CTTCTTGAACAACTTCTACC |
| | | | | | | CTGCTG | GCCAGTCAGAACAT | CCAAAGACATCAATGTCAA |
| | | | | | | TTCTGC | TATTGTTTGGTTAAA | GTGGAAGATTGATGGCAGT |
| | | | | | | TTTTTA | CTGGTACCAGCAGA | GAACGACAAAATGGCGTCC |
| | | | | | | GGTGTG | AACCAGGAAATATT | TGAACAGTTGGACTGATCAG |
| | | | | | | AGATGT | CCTAAACTATTGATC | GACAGCAAAGACAGCACCT |
| | | | | | | (SEQ ID | TATAAGGCTTCCAA | ACAGCATGAGCAGCACCCT |
| | | | | | | NO: 27) | CTTGCACACAGGCG | CACGTTGACCAAGGACGAG |
| | | | | | | | TCCCATCAAGGTTTA | TATGAACGACATAACAGCT |
| | | | | | | | GTGGCAGTGGATCT | ATACCTGTGAGGCCACTCAC |
| | | | | | | | GGAACAGGTTTCAC | AAGACATCAACTTCACCCAT |
| | | | | | | | ATTGACCATCAGCA | TGTCAAGAGCTTCAACAGG |
| | | | | | | | GCCTGCAGCCTGAA | AATGAGTGT (SEQ ID |
| | | | | | | | GACATTGCCACTTA | NO: 41) |
| | | | | | | | CTACTGTCAACAGG | |
| | | | | | | | GTCAAAGTTATCAC | |
| | | | | | | | ACGTGGACGTTCGG | |
| | | | | | | | TGGAGGCACCAAGC | |
| | | | | | | | TGGAAATCAAA | |
| | | | | | | | (SEQ ID NO: 36) | |
| cAb1 | 2293.254. | 1 | 1 | Mouse | Complete | ATGGAA | GACATTGTGATGAC | CGGGCTGATGCTGCACCAAC |
| 0205 | 030 | | | kappa | | TCACAG | ACAGTCTCCATCCTC | TGTATCCATCTTCCCACCAT |
| | (Fel d1 | | | | | ACTCAG | CCTGACTGTGACAA | CCAGTGAGCAGTTAACATCT |
| | MAb9) | | | | | GTCCTC | CAGGAGAGAAGGTC | GGAGGTGCCTCAGTCGTGTG |
| | | | | | | ATGTCC | ACTATGAACTGCAA | CTTCTTGAACAACTTCTACC |
| | | | | | | CTGCTG | GTCCAGTCAGAGTC | CCAAAGACATCAATGTCAA |
| | | | | | | TTCTGG | TGTTAAACAGTGCA | GTGGAAGATTGATGGCAGT |
| | | | | | | GTATCT | AATCAAAAGAACTA | GAACGACAAAATGGCGTCC |
| | | | | | | GGTACC | CTTGACCTGGCACC | TGAACAGTTGGACTGATCAG |
| | | | | | | TGTGGG | AGCAGAAACCAGGT | GACAGCAAAGACAGCACCT |
| | | | | | | (SEQ ID | CAGCCTCCTAAACT | ACAGCATGAGCAGCACCCT |
| | | | | | | NO: 28) | GTTGATCTACTGGG | CACGTTGACCAAGGACGAG |
| | | | | | | | CATCCACTAGGGAA | TATGAACGACATAACAGCT |
| | | | | | | | TCTGGGGTCCCTGAT | ATACCTGTGAGGCCACTCAC |
| | | | | | | | CGCTTCACAGGCAG | AAGACATCAACTTCACCCAT |
| | | | | | | | TGGATCTGGAACAG | TGTCAAGAGCTTCAACAGG |
| | | | | | | | ATTTCACTCTCACCA | AATGAGTGT (SEQ ID |
| | | | | | | | TCAGCAGTGTGCAG | NO: 41) |
| | | | | | | | GCTGAAGACCTGGC | |
| | | | | | | | AGTTTATTACTGTCA | |
| | | | | | | | GAATGATTATAGTTT | |
| | | | | | | | TCCATTCACGTTCGG | |
| | | | | | | | CTCGGGGACAAAGT | |
| | | | | | | | TGGAAATAAAA | |
| | | | | | | | (SEQ ID NO: 37) | |
| cAb1 | 2297.167. | 1 | 1 | Mouse | Complete | ATGAGC | GACATCCAGATGAC | CGGGCTGATGCTGCACCAAC |
| 0206 | 064 | | | kappa | | GTGCTC | TCAGTCTCCAGCCTC | TGTATCCATCTTCCCACCAT |
| | (Fel d1 | | | | | ACTCAG | CCTATCTGCATCTGT | CCAGTGAGCAGTTAACATCT |
| | MAb18) | | | | | GTCCTG | GGGAGAAACTGTCA | GGAGGTGCCTCAGTCGTGTG |
| | | | | | | GCGTTG | CCATCACATGTCGA | CTTCTTGAACAACTTCTACC |
| | | | | | | CTGCTG | GCAAGTGGGAATAT | CCAAAGACATCAATGTCAA |
| | | | | | | CTGTGG | TCACAATTATTTAGC | GTGGAAGATTGATGGCAGT |
| | | | | | | CTTACA | ATGGTTTCAGCAGA | GAACGACAAAATGGCGTCC |
| | | | | | | GGTGCC | GACAGGGAAAATCT | TGAACAGTTGGACTGATCAG |
| | | | | | | AGATGT | CCTCAGCTCCTGGTC | GACAGCAAAGACAGCACCT |
| | | | | | | (SEQ ID | TATAATGCAGAAAC | ACAGCATGAGCAGCACCCT |
| | | | | | | NO: 29) | CTTAGCAGATGGTG | CACGTTGACCAAGGACGAG |
| | | | | | | | TGCCATCAAGGTTC | TATGAACGACATAACAGCT |
| | | | | | | | AGTGGCAGTGGATC | ATACCTGTGAGGCCACTCAC |
| | | | | | | | AGGAACACAATATT | AAGACATCAACTTCACCCAT |
| | | | | | | | CTCTCAAGATCAGC | TGTCAAGAGCTTCAACAGG |
| | | | | | | | AGCCTGCAGCCTGA | AATGAGTGT (SEQ ID |
| | | | | | | | GGACTTTGGGAGTT | NO: 41) |
| | | | | | | | ATTTCTGTCAACATT | |
| | | | | | | | TTTGGAGTAGACCT | |
| | | | | | | | CCGACGTTCGGTGG | |
| | | | | | | | AGGCACCAAGCTGG | |
| | | | | | | | AAATCAAG (SEQ ID | |
| | | | | | | | NO: 38) | |
| cAb1 | 2297.179. | 2 | 1 | Mouse | Complete | ATGGAA | AACATTGTAATGAC | CGGGCTGATGCTGCACCAAC |
| 0207 | 089 | | | kappa | | TCACAG | CCAATCTCCCAAAT | TGTATCCATCTTCCCACCAT |
| | (Fel d1 | | | | | ACTCTG | CCATGTCCATGTCA | CCAGTGAGCAGTTAACATCT |
| | MAb20) | | | | | GTCTTC | GTAGGAGAGAGGGT | GGAGGTGCCTCAGTCGTGTG |
| | | | | | | ATATCC | CACCTTGAGCTGCA | CTTCTTGAACAACTTCTACC |
| | | | | | | ATACTG | AGGCCAGTGAGAAT | CCAAAGACATCAATGTCAA |
| | | | | | | CTCTGG | GTGGGTAGTTATGT | GTGGAAGATTGATGGCAGT |
| | | | | | | TTATAT | ATTCTGGTATCAAC | GAACGACAAAATGGCGTCC |
| | | | | | | GGTGCT | AGAAACCAGACCAG | TGAACAGTTGGACTGATCAG |
| | | | | | | GATGGG | TCTCCTAAACTGCTG | GACAGCAAAGACAGCACCT |
| | | | | | | (SEQ ID | ATATACGGGGCATC | ACAGCATGAGCAGCACCCT |
| | | | | | | NO: 30) | CAACCGGTACACTG | CACGTTGACCAAGGACGAG |
| | | | | | | | GGGTCCCCGATCGC | TATGAACGACATAACAGCT |
| | | | | | | | TTCACAGGCAGTAG | ATACCTGTGAGGCCACTCAC |
| | | | | | | | ATCTGCAACAGATT | AAGACATCAACTTCACCCAT |
| | | | | | | | TCACTCTGACCATCA | TGTCAAGAGCTTCAACAGG |
| | | | | | | | GCAATGTGCAGGCT | AATGAGTGT (SEQ ID |
| | | | | | | | GAAGACCTTGCAGA | NO: 41) |
| | | | | | | | TTATCACTGTGGAC | |
| | | | | | | | AGAGTTACAGCTAT | |
| | | | | | | | CCGCTCACGTTCGGT | |
| | | | | | | | GCTGGGACCAAGCT | |
| | | | | | | | GGAGCTGAAA (SEQ | |
| | | | | | | | ID NO: 39) | |

TABLE 13
Complementary Determining Regions (CDRs) of Antibodies or Antigen-binding fragments thereof of the present disclosure.

| CAB | Clone | | | CDR- | CDR- | CDR- | | CDR- | CDR- | CDR- |
|---|---|---|---|---|---|---|---|---|---|---|
| ID | Name | Scheme | Primary VH sequence | H1 | H2 | H3 | Primary VL sequence | L1 | L2 | L3 |
| cAb1 | 2291.014. | Kabat | EVKLMESGGGLVK | SYDMS | TISSGG | LLRTY | DIQMNQSPSSLSASL | HASQN | KASNL | QQGQS |
| 0204 | 055.067 | | PGGSLKLSCAASGF | (SEQ ID | TYTYY | AMDY | GDTITITCHASQNIIV | IIVWL | HT | YHTW |
| | (fel d1 | | TFSSYDMSWVRQTP | NO: 42) | PDSLK | (SEQ ID | WLNWYQQKPGNIP | N (SEQ | (SEQ ID | T (SEQ |
| | MAb2) | | EKRLEWVATISSGG | | G (SEQ | NO: 50) | KLLIYKASNLHTGV | ID NO: | NO: 58) | ID NO: |
| | | | TYTYYPDSLKGRFTI | | ID NO: | | PSRFSGSGSGTGFTL | 54) | | 62) |
| | | | SRDNARNTLYLQM | | 46) | | TISSLQPEDIATYYC | | | |
| | | | SSLRSEDTALFYCA | | | | QQGQSYHTWTFGG | | | |
| | | | RLLRTYAMDYWGQ | | | | GTKLEIK (SEQ ID | | | |
| | | | GTSVTVSS (SEQ ID | | | | NO: 15) | | | |
| | | | NO: 10) | | | | | | | |
| cAb1 | 2293.254. | Kabat | QVQLQQPGAELVKP | SYWM | EINPSN | RATMI | DIVMTQSPSSLTVTT | KSSQS | WASTR | QNDYS |
| 0205 | 030 | | GASVKLSCKASGYT | Y (SEQ | GRTNY | GGFAY | GEKVTMNCKSSQSL | LLNSA | ES | FPFT |
| | (Fel d1 | | FTSYWMYWVKQRP | ID NO: | NEKFK | (SEQ ID | LNSANQKNYLTWH | NQKN | (SEQ ID | (SEQ ID |
| | MAb9) | | GQGLEWIGEINPSN | 43) | T (SEQ | NO: 51) | QQKPGQPPKLLIYW | YLT | NO: 59) | NO: 63) |
| | | | GRTNYNEKFKTKAT | | ID NO: | | ASTRESGVPDRFTG | (SEQ ID | | |
| | | | LTVDKSSSTAYMQL | | 47) | | SGSGTDFTLTISSVQ | NO: 55) | | |
| | | | SSLTSEDSAVYYCA | | | | AEDLAVYYCONDY | | | |
| | | | RRATMIGGFAYWG | | | | SFPFTFGSGTKLEIK | | | |
| | | | QGTLVTVSA (SEQ | | | | (SEQ ID NO: 16) | | | |
| | | | ID NO: 11) | | | | | | | |
| cAb1 | 2297.167. | Kabat | QGQMQQSGAELVK | SSYIS | WIYAG | HYGNP | DIQMTQSPASLSAS | RASGN | NAETL | QHFWS |
| 0206 | 064 | | PGASVKLSCKTSGF | (SEQ ID | TGGTT | PAWFA | VGETVTITCRASGNI | IHNYL | AD | RPPT |
| | (Fel d1 | | TFSSSYISWLKQKPG | NO: 44) | YNQRF | Y (SEQ | HNYLAWFQQRQGK | A (SEQ | (SEQ ID | (SEQ ID |
| | MAb18) | | QSLEWIAWIYAGTG | | TG | ID NO: | SPQLLVYNAETLAD | ID NO: | NO: 60) | NO: 64) |
| | | | GTTYNQRFTGKAQL | | (SEQ ID | 52) | GVPSRFSGSGSGTQ | 56) | | |
| | | | TVDSSSSTAYMHFS | | NO: 48) | | YSLKISSLQPEDFGS | | | |
| | | | SLTTDDSAIYYCAR | | | | YFCQHFWSRPPTFG | | | |
| | | | HYGNPPAWFAYWG | | | | GGTKLEIK (SEQ ID | | | |
| | | | QGTLVTVSA (SEQ | | | | NO: 17) | | | |
| | | | ID NO: 12) | | | | | | | |
| cAb1 | 2297.179. | Kabat | QVQLQQPGTELVRP | SYWIN | NIYPS | RGNYG | NIVMTQSPKSMSMS | KASEN | GASNR | GQSYS |
| 0207 | 089 | | GASVKLSCKASGYT | (SEQ ID | DSYTN | NLYYF | VGERVTLSCKASEN | VGSYV | YT | YPLT |
| | (Fel d1 | | FTSYWINWVKQRP | NO: 45) | YNQKF | DY | VGSYVFWYQQKPD | F (SEQ | (SEQ ID | (SEQ ID |
| | MAb20) | | GQGLEWIGNIYPSD | | KD | (SEQ ID | QSPKLLIYGASNRY | ID NO: | NO: 61) | NO: 65) |
| | | | SYTNYNQKFKDKA | | (SEQ ID | NO: 53) | TGVPDRFTGSRSAT | 57) | | |
| | | | TLTVDKSSSTAYMQ | | NO: 49) | | DFTLTISNVQAEDL | | | |
| | | | LSSPTSDDSAVYYC | | | | ADYHCGQSYSYPLT | | | |
| | | | TRRGNYGNLYYFD | | | | FGAGTKLELK (SEQ | | | |
| | | | YWGQGTTLTVSS | | | | ID NO: 18) | | | |
| | | | (SEQ ID NO: 13) | | | | | | | |

TABLE 14
Heavy chain and light chains of Antibodies or Antigen-binding fragments thereof of the present
disclosure

| cAb | Clone | | |
|---|---|---|---|
| ID | Name | Heavy Chain | Light Chain |
| cAb1 | 2291.014. | EVKLMESGGGLVKPGGSLKLSCAASGFTFSSYDMSWVRQ | DIQMNQSPSSLSASLGDTITITCHASQNIIVWLNW |
| 0204 | 055.067 | TPEKRLEWVATISSGGTYTYYPDSLKGRFTISRDNARNTLY | YQQKPGNIPKLLIYKASNLHTGVPSRFSGSGSGTG |
| | (fel d1 | LQMSSLRSEDTALFYCARLLRTYAMDYWGQGTSVTVSSA | FTLTISSLQPEDIATYYCQQGQSYHTWTFGGGTKL |
| | MAb2) | KTTPPSVYPLAPGSAAQTNSMVTLGCLVKGYFPEPVTVTW | EIKRADAAPTVSIFPPSSEQLTSGGASVVCFLNNF |
| | | NSGSLSSGVHTFPAVLQSDLYTLSSSVTVPSSTWPSETVTC | YPKDINVKWKIDGSERQNGVLNSWTDQDSKDSTYS |
| | | NVAHPASSTKVDKKIVPRDCGCKPCICTVPEVSSVFIFPPK | MSSTLTLTKDEYERHNSYTCEATHKTSTSPIVKSF |
| | | PKDVLTITLTPKVTCVVVDISKDDPEVQFSWFVDDVEVHTA | NRNEC (SEQ ID NO: 72) |
| | | QTQPREEQFNSTFRSVSELPIMHQDWLNGKEFKCRVNSAA | |
| | | FPAPIEKTISKTKGRPKAPQVYTIPPPKEQMAKDKVSLTCM | |
| | | ITDFFPEDITVEWQWNGQPAENYKNTQPIMDTDGSYFVYS | |
| | | KLNVQKSNWEAGNTFTCSVLHEGLHNHHTEKSLSHSPGK | |
| | | (SEQ ID NO: 67) | |
| cAb1 | 2293.254. | QVQLQQPGAELVKPGASVKLSCKASGYTFTSYWMYWVK | DIVMTQSPSSLTVTTGEKVTMNCKSSQSLLNSANQ |
| 0205 | 030 | QRPGQGLEWIGEINPSNGRTNYNEKFKTKATLTVDKSSST | KNYLTWHQQKPGQPPKLLIYWASTRESGVPDRFTG |
| | (Fel d1 | AYMQLSSLTSEDSAVYYCARRATMIGGFAYWGQGTLVTV | SGSGTDFTLTISSVQAEDLAVYYCONDYSFPFTFG |
| | MAb9) | SAAKTTPPSVYPLAPGSAAQTNSMVTLGCLVKGYFPEPVT | SGTKLEIKRADAAPTVSIFPPSSEQLTSGGASVVC |
| | | VTWNSGSLSSGVHTFPAVLQSDLYTLSSSVTVPSSTWPSET | FLNNFYPKDINVKWKIDGSERQNGVLNSWTDQDSK |
| | | VTCNVAHPASSTKVDKKIVPRDCGCKPCICTVPEVSSVFIF | DSTYSMSSTLTLTKDEYERHNSYTCEATHKTSTSP |
| | | PPKPKDVLTITLTPKVTCVVVDISKDDPEVQFSWFVDDVE | IVKSFNRNEC (SEQ ID NO: 73) |
| | | VHTAQTQPREEQFNSTFRSVSELPIMHQDWLNGKEFKCRV | |
| | | NSAAFPAPIEKTISKTKGRPKAPQVYTIPPPKEQMAKDKVS | |
| | | LTCMITDFFPEDITVEWQWNGQPAENYKNTQPIMDTDGSY | |
| | | FVYSKLNVQKSNWEAGNTFTCSVLHEGLHNHHTEKSLSH | |
| | | SPGK (SEQ ID NO: 68) | |
| cAb1 | 2297.167. | QGQMQQSGAELVKPGASVKLSCKTSGFTFSSSYISWLKQK | DIQMTQSPASLSASVGETVTITCRASGNIHNYLAW |
| 0206 | 064 | PGQSLEWIAWIYAGTGGTTYNQRFTGKAQLTVDSSSSTAY | FQQRQGKSPQLLVYNAETLADGVPSRFSGSGSGTQ |
| | (Fel d1 | MHFSSLTTDDSAIYYCARHYGNPPAWFAYWGQGTLVTVS | YSLKISSLQPEDFGSYFCQHFWSRPPTFGGGTKLE |
| | MAb18) | AAKTTPPSVYPLAPGSAAQTNSMVTLGCLVKGYFPEPVTV | IKRADAAPTVSIFPPSSEQLTSGGASVVCFLNNFY |
| | | TWNSGSLSSGVHTFPAVLQSDLYTLSSSVTVPSSTWPSETV | PKDINVKWKIDGSERQNGVLNSWTDQDSKDSTYSM |
| | | TCNVAHPASSTKVDKKIVPRDCGCKPCICTVPEVSSVFIFP | SSTLTLTKDEYERHNSYTCEATHKTSTSPIVKSFN |
| | | PKPKDVLTITLTPKVTCVVVDISKDDPEVQFSWFVDDVEVH | RNEC (SEQ ID NO: 74) |
| | | TAQTQPREEQFNSTFRSVSELPIMHQDWLNGKEFKCRVNS | |
| | | AAFPAPIEKTISKTKGRPKAPQVYTIPPPKEQMAKDKVSLT | |
| | | CMITDFFPEDITVEWQWNGQPAENYKNTQPIMDTDGSYF | |
| | | VYSKLNVQKSNWEAGNTFTCSVLHEGLHNHHTEKSLSHS | |
| | | PGK (SEQ ID NO: 69) | |
| cAb1 | 2297.179. | QVQLQQPGTELVRPGASVKLSCKASGYTFTSYWINWVKQ | NIVMTQSPKSMSMSVGERVTLSCKASENVGSYVFW |
| 0207 | 089 | RPGQGLEWIGNIYPSDSYTNYNQKFKDKATLTVDKSSSTA | YQQKPDQSPKLLIYGASNRYTGVPDRFTGSRSATD |
| | (Fel d1 | YMQLSSPTSDDSAVYYCTRRGNYGNLYYFDYWGQGTTL | FTLTISNVQAEDLADYHCGQSYSYPLTFGAGTKLE |
| | MAb20) | TVSSAKTTPPSVYPLAPGSAAQTNSMVTLGCLVKGYFPEP | LKRADAAPTVSIFPPSSEQLTSGGASVVCFLNNFY |
| | | VTVTWNSGSLSSGVHTFPAVLQSDLYTLSSSVTVPSSTWPS | PKDINVKWKIDGSERQNGVLNSWTDQDSKDSTYSM |
| | | ETVTCNVAHPASSTKVDKKIVPRDCGCKPCICTVPEVSSVF | SSTLTLTKDEYERHNSYTCEATHKTSTSPIVKSFN |
| | | IFPPKPKDVLTITLTPKVTCVVVDISKDDPEVQFSWFVDDV | RNEC (SEQ ID NO: 75) |
| | | EVHTAQTQPREEQFNSTFRSVSELPIMHQDWLNGKEFKCR | |
| | | VNSAAFPAPIEKTISKTKGRPKAPQVYTIPPPKEQMAKDKV | |
| | | SLTCMITDFFPEDITVEWQWNGQPAENYKNTQPIMDTDGS | |
| | | YFVYSKLNVQKSNWEAGNTFTCSVLHEGLHNHHTEKSLS | |
| | | HSPGK (SEQ ID NO: 70) | |
| cAb1 | 2297.179. | QIQLVQSGPELRKPGETVRISCKASGYTFTTYGMDWVKQA | NIVMTQSPKSMSMSVGERVTLSCKASENVGSYVFW |
| 0207 | 089 | PGKVLKWVGWINTNTGEPTYAEEFKGRFAFSLETSASTAY | YQQKPDQSPKLLIYGASNRYTGVPDRFTGSRSATD |
| | (Fel d1 | FQINNLKNEDTATYFCARKWLRRAMDYWGQGTSVTVSS | FTLTISNVQAEDLADYHCGQSYSYPLTFGAGTKLE |
| | MAb20- | AKTTPPSVYPLAPGSAAQTNSMVTLGCLV (SEQ ID | LKRADAAPTVSIFPPSSEQLTSGGASVVCFLNNFY |
| | 1) | NO: 71) | PKDINVKWKIDGSERQNGVLNSWTDQDSKDSTYSM |
| | | | SSTLTLTKDEYERHNSYTCEATHKTSTSPIVKSFN |
| | | | RNEC (SEQ ID NO: 75) |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification. In some aspects, technical publications are referenced by name and date within parenthesis. The full bibliographic citations for these references are incorporated herein by reference.

1. An isolated antibody or antigen-binding fragment thereof that binds to Fel d 1, wherein the antibody or antigen-binding fragment thereof comprises:
(a) a heavy chain complementary determining region 1 (CDRH1) comprising the amino acid sequence of any one of SEQ ID NOs: 42-45, a heavy chain complementary determining region 2 (CDRH2) comprising the amino acid sequence of any one of SEQ ID NOs: 46-49, and a heavy chain complementary determining region 3 (CDRH3) comprising the amino acid sequence of any one of SEQ ID NOs: 50-53; and
(b) a light chain complementary determining region 1 (CDRL1) comprising the amino acid sequence of any one of SEQ ID NOs: 54-57, a light chain complementary determining region 2 (CDRL2) comprising the amino acid sequence of any one of SEQ ID NOs: 58-61, and a light chain complementary determining region 3 (CDRL3) comprising the amino acid sequence of any one of SEQ ID NOs: 62-65.

2. The isolated antibody or antigen-binding fragment thereof of claim 1, wherein the antibody or antigen-binding fragment thereof comprises:
(a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62;
(b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63;
(c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or
(d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

3. The antibody or antigen-binding fragment thereof of claim 1, wherein the antibody or antigen-binding fragment thereof comprises a heavy chain variable domain sequence (VH) and a light chain variable domain sequence (VL) comprising at least 96%, at least 97%, at least 98%, or at least 99% identity to an amino acid sequence selected from:
(a) the amino acid sequence of SEQ ID NO: 10 and the amino acid sequence of SEQ ID NO: 15;
(b) the amino acid sequence of SEQ ID NO: 11 and the amino acid sequence of SEQ ID NO: 16;
(c) the amino acid sequence of SEQ ID NO: 12 and the amino acid sequence of SEQ ID NO: 17; or
(d) the amino acid sequence of SEQ ID NO: 13 and the amino acid sequence of SEQ ID NO: 18.

4. The antibody or antigen-binding fragment thereof of claim 1, wherein the antibody or antigen-binding fragment thereof comprises:
(a) the amino acid sequence of SEQ ID NO: 10 and the amino acid sequence of SEQ ID NO: 15;
(b) the amino acid sequence of SEQ ID NO: 11 and the amino acid sequence of SEQ ID NO: 16;
(c) the amino acid sequence of SEQ ID NO: 12 and the amino acid sequence of SEQ ID NO: 17; or
(d) the amino acid sequence of SEQ ID NO: 13 and the amino acid sequence of SEQ ID NO: 18.

5. The antibody or antigen-binding fragment thereof of claim 1, wherein the antibody or antigen-binding fragment thereof is a monoclonal antibody.

6. The antibody or antigen-binding fragment thereof of claim 1, wherein the antigen-binding fragment thereof is a recombinant single chain fragment variable or single chain variable domain fragment (scFv) antibody, a Fab fragment, a F(ab′)2 fragment, or a variable domain fragment (Fv fragment).

7. The antibody or antigen-binding fragment thereof of claim 6, wherein the antigen-binding fragment thereof is a recombinant antibody variable domain fragment (Fv fragment) and/or wherein the antigen-binding fragment thereof comprises the amino acid sequence of one or more of SEQ ID NOs: 10-18.

8. (canceled)

9. (canceled)

10. The antibody or antigen-binding fragment thereof of claim 1, wherein the antibody or antigen-binding fragment thereof is conjugated to a label.

11. (canceled)

12. An isolated nucleic acid comprising a nucleotide sequence encoding an antibody or antigen-binding fragment thereof that binds to Fel d 1, wherein the antibody or antigen-binding fragment thereof comprises:
(a) a heavy chain complementary determining region 1 (CDRH1) comprising the amino acid sequence of any one of SEQ ID NOs: 42-45, a heavy chain complementary determining region 2 (CDRH2) comprising the amino acid sequence of any one of SEQ ID NOs: 46-49, and a heavy chain complementary determining region 3 (CDRH3) comprising the amino acid sequence of any one of SEQ ID NOs: 50-53; and
(b) a light chain complementary determining region 1 (CDRL1) comprising the amino acid sequence of any one of SEQ ID NOs: 54-57, a light chain complementary determining region 2 (CDRL2) comprising the amino acid sequence of any one of SEQ ID NOs: 58-61, and a light chain complementary determining region 3 (CDRL3) comprising the amino acid sequence of any one of SEQ ID NOs: 62-65.

13. (canceled)

14. A vector comprising the isolated nucleic acid of claim 12.

15. (canceled)

16. A method of detecting or quantifying Fel d 1 in a sample comprising:
(a) contacting the sample with the antibody or antigen-binding fragment thereof of claim 1; and
(b) detecting Fel d 1 in the sample by detecting the binding of the antibody or the antigen-binding fragment thereof to the Fel d 1 in the sample.

17. The method of claim 16, wherein the sample is a biological sample isolated from a feline.

18. (canceled)

19. (canceled)

20. The method of claim 16, wherein the sample is an environmental sample.

21. The method of claim 16, wherein the detection comprises enzyme-linked immunosorbent assay (ELISA), lateral flow assay, immunohistochemistry, immunofluorescence, or Western blot.

22. A method of detecting or quantifying Fel d 1 in a sample comprising:
(a) contacting the sample with a first antibody or antigen-binding fragment thereof selected from an anti-Fel d 1 antibody or antigen-binding fragment thereof according to claim 1, thereby producing a first complex comprising the Fel d 1 and the first antibody or antigen-binding fragment thereof;
(b) contacting the first complex with a second antibody or antigen-binding fragment thereof selected from an anti-Fel d 1 antibody or antigen-binding fragment thereof according to claim 1, thereby producing a second complex comprising the Fel d 1, the first antibody or antigen-binding fragment thereof, and the second antibody or antigen-binding fragment thereof;
wherein either of the first antibody or antigen-binding fragment thereof or the second antibody or antigen-binding fragment thereof comprises a label capable of producing a signal; and
(c) measuring the signal.

23.-30. (canceled)

31. The method of claim 22, wherein the label comprises colloidal gold, horseradish peroxidase, or a dye.

32. The method of claim 31, wherein the dye comprises a fluorescent dye.

33. The method of claim 22, wherein the first antibody or antigen-binding fragment thereof comprises:
(a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62;
(b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63;
(c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or
(d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

34. The method of claim 22, wherein the second antibody or antigen-binding fragment thereof comprises:
(a) a CDRH1 comprising the amino acid sequence of SEQ ID NO: 42, a CDRH2 comprising the amino acid sequence of SEQ ID NO: 46, and a CDRH3 comprising the amino acid sequence of SEQ ID NO: 50, and a CDRL1 comprising the amino acid sequence of SEQ ID NO: 54, a CDRL2 comprising the amino acid sequence of SEQ ID NO: 58, and a CDRL3 comprising the amino acid sequence of SEQ ID NO: 62;
(b) a CDRH1 comprising the amino acid sequence of SEQ ID NO: 43, a CDRH2 comprising the amino acid sequence of SEQ ID NO: 47, and a CDRH3 comprising the amino acid sequence of SEQ ID NO: 51, and a CDRL1 comprising the amino acid sequence of SEQ ID NO: 55, a CDRL2 comprising the amino acid sequence of SEQ ID NO: 59, and a CDRL3 comprising the amino acid sequence of SEQ ID NO: 63;
(c) a CDRH1 comprising the amino acid sequence of SEQ ID NO: 44, a CDRH2 comprising the amino acid sequence of SEQ ID NO: 48, and a CDRH3 comprising the amino acid sequence of SEQ ID NO: 52, and a CDRL1 comprising the amino acid sequence of SEQ ID NO: 56, a CDRL2 comprising the amino acid sequence of SEQ ID NO: 60, and a CDRL3 comprising the amino acid sequence of SEQ ID NO: 64; or
(d) a CDRH1 comprising the amino acid sequence of SEQ ID NO: 45, a CDRH2 comprising the amino acid sequence of SEQ ID NO: 49, and a CDRH3 comprising the amino acid sequence of SEQ ID NO: 53, and a CDRL1 comprising the amino acid sequence of SEQ ID NO: 57, a CDRL2 comprising the amino acid sequence of SEQ ID NO: 61, and a CDRL3 comprising the amino acid sequence of SEQ ID NO: 65.

35. A kit comprising one or more antibodies or antigen-binding fragments thereof of claim 1.

36.-39. (canceled)
